(19) United States
(12) Patent Application Publication
(10) Pub. No.: US 2003/0039661 A1
(43) Pub. Date: Feb. 27, 2003

(54) Methods, compositions and kits for preserving antigenicity

(76) Inventors: Teresa Aja, Imperial Beach, CA (US); Brett W. Ching, San Diego, CA (US); Patricia L. Gladstone, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/087,607

(22) Filed: Mar. 1, 2002

Related U.S. Application Data
(60) Provisional application No. 60/272,750, filed on Mar. 2, 2001.

(51) Int. Cl. A61K 39/12; A61K 39/21; A61K 39/29

(52) U.S. Cl. 424/186.1; 424/188.1; 424/189.1

(57) ABSTRACT

The present invention relates generally to programmed cell death and specifically to methods, compositions, and kits for preserving or enhancing antigenicity of markers associated with disease by utilizing inhibitors of apoptosis including interleukin-1β-converting enzyme (ICE)/CED-3 family inhibitors.

### CROSS-REFERENCE TO RELATED APPLICATION

This application claims the benefit of U.S. Provisional Patent Application No. 60/272,750 filed Mar. 2, 2001, which provisional application is incorporated herein by reference in its entirety.

### TECHNICAL FIELD

The present invention relates generally to programmed cell death and specifically to methods, compositions, and kits for preserving or enhancing antigenicity of markers associated with disease by utilizing inhibitors of apoptosis including interleukin-1β-converting enzyme (ICE)/CED-3 family inhibitors.

### BACKGROUND OF THE INVENTION

The present invention relates generally to programmed cell death and specifically to methods, compositions, and kits for preserving or enhancing antigenicity of markers associated with disease by utilizing inhibitors of apoptosis including interleukin-1β-converting enzyme (ICE)/CED-3 family inhibitors.

Necrosis and apoptosis are two basic processes by which cells may die. In necrosis cell death usually is a result of cell injury. The cells generally swell and lyse, and the cell contents ultimately spill into the extracellular space. By contrast, apoptosis is a mode of cell death in which single cells are deleted in the midst of living tissues. Apoptosis accounts for most of the programmed cell death in tissue remodeling and for the cell loss that accompanies atrophy of adult tissues following withdrawal of endocrine and other growth stimuli. In addition, apoptosis is believed to be responsible for the physiologic death of cells in the course of normal tissue turnover (i.e., tissue homeostasis) (Kerr, J. F. et al., Br. J. Cancer 26:239-257 (1972); Wyllie, A. H. et al., Int. Rev. Cytol. 68:251-306 (1980)).

Apoptosis is known to be involved in a variety of disease states, including infectious disease. Most bacterial and viral infections modulate apoptosis in one way or another. While many infections lead to induction of apoptosis, still other infections inhibit apoptosis to facilitate the replicative cycle of the infectious agent. One consequence of viral or bacterial infection is the initiation of an apoptotic event to protect the organism from cells or areas of the organism that are infected. However, some infectious agents such as, several viruses, encode inhibitors of various apoptotic proteins.

In various cell culture systems, it has been shown that inhibition of ICE/CED-3 family members can effectively inhibit apoptosis. For example, the compound acetyl-DEVD-aldehyde inhibited anti-Fas induced apoptosis in a T-lymphocyte cell line (Schlegel et al., J. Biol. Chem. 271:1841, (1996); Enari et al., Nature, 380:723,1996). Similarly, acetyl-AD-aldehyde and acetyl-YVAD-chloromethylketone blocked the death of motoneurons in vitro and in vivo (Milligan et al., Neuron, 15:385 (1995)). In addition, the ICE/CED-3 family inhibitor Boc-D-(benzyl) chloromethylketone as well as crmA prevented the cell death of mammary epithelial cells that occurs in the absence of extracellular matrix (Boudreau et al., Science, 27:891, (1995)).

It is known that control of apoptosis may have utility in treating disease. Specifically, inhibitors of the ICE/CED-3 family may have therapeutic effects. For example, it has been suggested that inhibition of ICE may be useful in the treatment of inflammatory disorders (Dolle et al., J. Med. Chem., 37:563, (1994); Thornberry et al., Biochemistry, 33:3934, (1994)). It is also known that inhibitors of ICE/CED-3 family members may have utility in treating degenerative diseases such as neurodegenerative diseases (e.g., Alzheimer's disease, Parkinson's disease, amyotrophic lateral sclerosis, Huntington's disease), ischemic disease of heart or central nervous system (i.e., myocardial infarction and stroke), and traumatic brain injury, as well as in alopecia, AIDS and toxin induced liver disease (Nicholson, Nature Biotechnology 14:297, 1996).

### SUMMARY OF THE INVENTION

The present invention is directed to methods, compositions and kits for preserving and/or enhancing antigen detection of infected tissue samples by preventing the programmed death of cells through inhibiting the activity of proteases of the interleukin-1β-converting enzyme (ICE)/CED-3 family (referred to commonly as Caspases). The current invention provides new methods for using such inhibitors and is centered upon the surprising finding that infected tissue samples contacted with inhibitors of apoptosis maintain antigen presentation for substantial periods of time, thus allowing for longer periods of time between collection and processing. Delays in processing can be advantageous for several reasons. For instance, the site of collection may not be located near a testing facility, the test may be prohibitively expensive when not performed at a centralized testing facility, or specially trained technicians who are not available near the collection site may be required to perform the test.

In one aspect the present invention provides methods for preserving antigen presentation on a virally infected mammalian cell, comprising providing a population of mammalian cells at least a portion of which is suspected of being virally infected and contacting said cells with an anti-apoptotic reagent, thereby preserving antigen presentation on virally infected cells.

In certain embodiments the cells comprise peripheral blood leukocytes. In related embodiments the cells may comprise neutrophils. In yet other related embodiments the cells may comprise granulocytes.

In certain aspects the virus to be detected may be herpes, HIV, cytomegalovirus (CMV), hepatitis or the like.

In the various aspects the antigen comprises a viral antigen present on the surface of the mammalian cells. In related embodiments, the antigen comprises the pp65 protein of CMV.

In yet other embodiments contacting of the cells occurs ex vivo. While in other embodiments the reagent is a nucleic acid, such as an antisense sequence. In yet other embodiments, the reagent is a protease inhibitor, that is either reversible or irreversible. Such protease inhibitors may be obtained from variety of sources. In other embodiments, the protease inhibitor is an inhibitor of the ICE/Ced-3 family of proteases.

In specific embodiments the following compounds may be used within the context of the present invention:

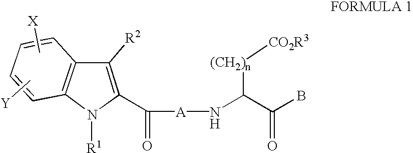

wherein:

n is 1 or 2;.

R¹ is alkyl, cycloalkyl, (cycloalkyl)alkyl, phenyl, (substituted)phenyl, phenylalkyl, (substituted)phenylalkyl, heteroaryl, (heteroaryl)alkyl or (CH₂)ₘCO₂R⁴, wherein m=1-4, and R⁴ is as defined below;

R² is a hydrogen atom, chloro, alkyl, cycloalkyl, (cycloalkyl)alkyl, phenyl, (substituted)phenyl, phenylalkyl, (substituted)phenylalkyl, heteroaryl, (heteroaryl)alkyl or (CH₂)ₚCO₂R⁵, wherein p=0-4, and R⁵ is as defined below;

R³ is a hydrogen atom, alkyl, cycloalkyl, (cycloalkyl)alkyl, phenylalkyl, or (substituted)phenylalkyl;

R⁴ is a hydrogen atom, alkyl, cycloalkyl, (cycloalkyl)alkyl, phenylalkyl, or (substituted)phenylalkyl;

R⁵ is a hydrogen atom, alkyl, cycloalkyl, (cycloalkyl)alkyl, phenylalkyl, or (substituted)phenylalkyl;

A is a natural and unnatural amino acid;

B is a hydrogen atom, a deuterium atom, alkyl, cycloalkyl, (cycloalkyl)alkyl, phenyl, (substituted)phenyl, phenylalkyl, (substituted)phenylalkyl, heteroaryl, (heteroaryl)alkyl, halomethyl, CH₂ZR⁶, CH₂OCO(aryl), CH₂OCO(heteroaryl); or CH₂OPO(R⁷)R⁸, where Z is an oxygen or a sulfur atom;

R⁶ is phenyl, substituted phenyl, phenylalkyl, substituted phenylalkyl, heteroaryl, or (heteroaryl)alkyl; and

R⁷ and R⁸ are independently selected from a group consisting of alkyl, cycloalkyl, phenyl, substituted phenyl, phenylalkyl, (substituted phenyl) alkyl, and (cycloalkyl) alkyl; and

X and Y are independently selected from the group consisting of a hydrogen atom, halo, trihalomethyl, amino, protected amino, an amino salt, mono-substituted amino, di-substituted amino, carboxy, protected carboxy, a carboxylate salt, hydroxy, protected hydroxy, a salt of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl;

or a pharmaceutically acceptable salt thereof.

In addition, the following compounds may be used in the context of the present invention:

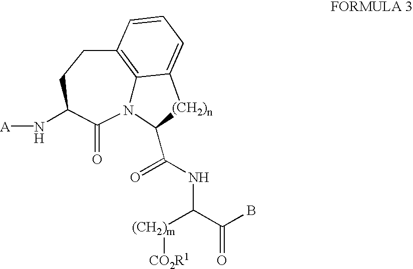

wherein:

n is 1 or 2;

m is 1 or 2;

A is R²CO—, R³—O—CO—, or R⁴SO₂—,

a group of the formula:

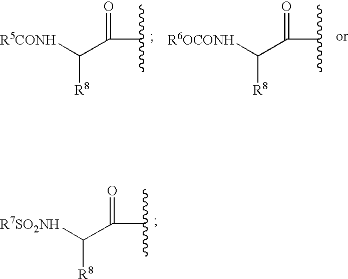

further wherein:

R¹ is a hydrogen atom, alkyl or phenylalkyl;

R² is alkyl, cycloalkyl, (cycloalkyl)alkyl, phenyl, phenylalkyl, substituted phenyl, (substituted phenyl)alkyl, heteroaryl, or (heteroaryl)alkyl;

R³ is alkyl, cycloalkyl, (cycloalkyl)alkyl, phenylalkyl, or (substituted phenyl)alkyl;

R⁴ is alkyl, cycloalkyl, (cycloalkyl)alkyl, phenyl, phenylalkyl, substituted phenyl, (substituted phenyl)alkyl, heteroaryl, or (heteroaryl)alkyl;

R⁵ is alkyl, cycloalkyl, (cycloalkyl)alkyl, phenyl, phenylalkyl, substituted phenyl, (substituted phenyl)alkyl, heteroaryl, or (heteroaryl)alkyl;

R⁶ is alkyl, cycloalkyl, (cycloalkyl)alkyl, phenylalkyl, or (substituted phenyl)alkyl;

R⁷ is alkyl, cycloalkyl, (cycloalkyl)alkyl, phenyl, phenylalkyl, substituted phenyl, (substituted phenyl)alkyl, heteroaryl, or (heteroaryl)alkyl;

R⁸ is an amino acid side chain chosen from the group consisting of natural and unnatural amino acids;

B is a hydrogen atom, a deuterium atom, alkyl, cycloalkyl, (cycloalkyl)alkyl, phenyl, phenylalkyl, substituted phenyl, (substituted phenyl)alkyl, heteroaryl, (heteroaryl)alkyl, or halomethyl;

a group of the formula:

—CH₂XR⁹;

wherein R⁹ is phenyl, substituted phenyl, phenylalkyl, (substituted phenyl)alkyl, heteroaryl, or (heteroaryl)alkyl; and X is an oxygen or a sulfur atom;

a group of the formula:

—CH₂—O—CO-(aryl);

a group of the formula:

—CH₂—O—CO-(heteroaryl);

a group of the formula:

—CH₂—O—PO(R¹⁰)R¹¹

wherein R¹⁰ and R¹¹ are independently selected from a group consisting of alkyl, cycloalkyl, phenyl, substituted phenyl, phenylalkyl and (substituted phenyl) alkyl; and the pharmaceutically-acceptable salts thereof.

In addition, the following compounds may be used in the context of the present invention:

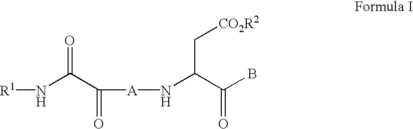

wherein:

A is a natural or unnatural amino acid of Formula IIa-i:

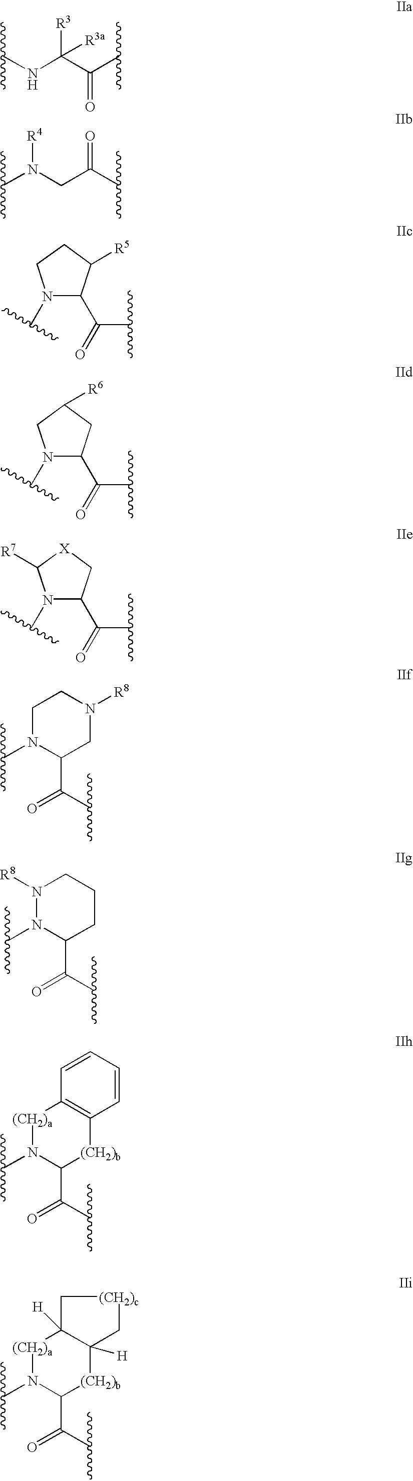

B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₙcycloalkyl, (CH₂)ₙphenyl, (CH₂)ₙ(substituted phenyl), (CH₂)ₙ(1 or 2-naphthyl), (CH₂)ₙ(heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c:

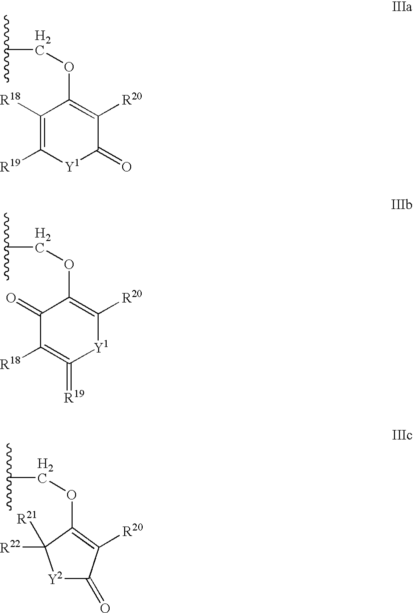

R¹ is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, (heteroaryl)alkyl, R^{1a}(R^{1b})N, [or] R^{1c}O, 2-phenoxyphenyl or 2- or 3-benzylphenyl; and

R² is hydrogen, lower alkyl, cycloalkyl, (cycloalkyl)alkyl, phenylalkyl, or substituted phenylalkyl;

and wherein:

R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl, with the proviso that R^{1a} and R^{1b} cannot both be hydrogen;

R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl;

R³ is C₁₋₆ lower alkyl, cycloalkyl, phenyl, substituted phenyl, (CH₂)ₙNH₂, (CH₂)ₙNHCOR⁹, (CH₂)ₙN(C═NH)NH₂, (CH₂)ₘCO₂R², (CH₂)ₘOR¹⁰, (CH₂)ₘSR¹¹,(CH₂)ₙcycloalkyl,(CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;

R^{3a} is hydrogen or methyl, or R³ and R^{3a} taken together are —(CH₂)_{d}— where d is an integer from 2 to 6;

R⁴ is phenyl, substituted phenyl, (CH₂)ₘphenyl, (CH₂)ₘ(substituted phenyl), cycloalkyl, or benzofused cycloalkyl;

R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);

R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹ or NHCOR⁹;

R⁷ is hydrogen, oxo, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR^{9;}

R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R¹⁴;

R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);

R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);

R¹⁴ is hydrogen or lower alkyl;

or R¹³ and R¹⁴ taken together form a five to seven membered carbocyclic or heterocyclic ring, such as morpholine, or N-substituted piperazine;

R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);

R¹⁶ and R¹⁷ are independently lower alkyl, cycloalkyl, phenyl, substituted phenyl, naphthyl, phenylalkyl, substituted phenylalkyl, or (cycloalkyl)alkyl;

R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂—;

R²⁰ is hydrogen, alkyl, phenyl, substituted phenyl, (CH₂)ₙphenyl, (CH₂)ₙ(substituted phenyl);

R²¹, R²² and R²³ are independently hydrogen, or alkyl;

X is CH₂, (CH₂)₂, (CH₂)₃, or S;

Y¹ is O or NR²³;

Y² is CH₂, O, or NR²³;

a is 0 or 1 and b is 1 or 2, provided that when a is 1 then b is 1;

c is 1 or 2, provided that when c is 1 then a is 0and b is 1;

m is 1 or 2; and

n is 1,2, 3 or 4;

or a pharmaceutically acceptable salt thereof.

In certain embodiments the protease inhibitor may be one of the exemplary compounds useful as ICE/CED-3 inhibitors included herein. Such compounds and method of synthesis are described in their entirety in co-pending PCT publications and U.S. Patent Applications: 09/550,917; 09/482,813; WO 00/23421 (related U.S. Application No. 09/177,546); WO 01/00658 (related U.S. application Ser. Nos. 09/260,816 and 09/345,724) and WO 00/01666 (related U.S. application Ser. No. 09/177,549), incorporated by reference in their entirety.

Other peptide and peptidyl inhibitors of ICE have been described in various patent applications and issued patents, as have a variety of other inhibitors which are herein incorporated by reference in their entirety (see, e.g., U.S. Pat. Nos. 6,187,771; 5,968,927; 5,567,425; 6,004,579; 6,004,933; 5,798,247; 5,867,519; 5,877,197; 5,756,465; 5,416,013; 6,153,591; 6,136,787; 6,184,210; 6,083,981; 5,919,790; 5,635,187; 5,635,186; and 5,624,672; In addition this application claims priority to WO 00/20440 (related to U.S. Provisional Application No. 60/103,428); WO 01/10383 (related to U.S. Provisional Application No. 60/147,206); WO 00/64430; WO 99/65451 (related U.S. Provisional Application No. 60/089,723); WO 97/22619 (related U.S. application Ser. Nos. 08/575,641; 08/598,332; 08/712,878; 60/031,495; 08/761,483); WO 97/07805 (related U.S. Provisional Application No. 60/003,083); WO 97/08174 (related U.S. Provisional Application No. 60/003,082);

WO 98/24804 (related U.S. Application Nos. 60/032,129; 60/041,938; and 60/050,796); WO 98/24805 (related U.S. Application Nos. 60/032,792; 60/042,660; and 60/053,001);

WO 98/22098 (related U.S. application Ser. No. 08/754,491); WO 99/46248 (related U.S. Provisional Application No. 60/077,327); WO 99/43675 (related U.S. application Ser. No. 09/030,975); WO 99/25346 (related U.S. application Ser. No. 09/317,926); WO 99/06042 (related U.S. Provisional Application No. 60/054,249); WO 99/06367 (related U.S. Provisional Application No. 60/054,255); WO 97/22618 (related U.S. application Ser. No. 08/575,648); and WO 97/47545 (related U.S. Provisional Application No. 60/078,770).

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 sets forth the activity of the compounds in Formula A in inhibiting the activity of CASPASE-1 and CASPASE-3 enzymes.

FIG. 2 illustrates the activity of the compounds in Formula B with respect to recombinant CASPASE-1, CASPASE-3, CASPASE-6 and CASPASE-8 enzymes.

FIG. 3 illustrates the activity of the compounds in Formula C with respect to recombinant CASPASE-1, CASPASE-3, CASPASE-6 and CASPASE-8 enzymes.

FIG. 4 sets forth the activity of compounds in Formula D in inhibiting the activity of CASPASE-1, CASPASE-3, CASPASE-6, CASPASE-7 and CASPASE-8 enzymes.

FIG. 5 sets forth the activity of Example 106 in inhibiting the activity of CASPASE-1, CASPASE-3, CASPASE-6 and CASPASE-8.

FIG. 6 shows results derived from FACS analysis demonstrating the effect of ICE/CED-3 inhibitors on neutrophil survival as measured by DNA content (% hypodiploid).

FIG. 7 shows the effect of ICE/CED-3 inhibitors or neutrophil survival as measured by the ability of live neutrophils to undergo oxidative burst.

### DETAILED DESCRIPTION OF THE INVENTION

Prior to setting forth the invention, it may be helpful to an understanding thereof to set forth definitions of certain terms that will be used hereinafter.

As used herein, a “caspase” and “ICE/Ced-3 family of proteases” are used interchangeable herein and refer to a cysteine protease that specifically cleaves proteins after Asp residues. Caspases are initially expressed as zymogens, in which a large subunit is N-terminal to a small subunit. Caspases are generally activated by cleavage at internal Asp residues. These proteins have been identified in many eukaryotes, including C. elegans, Drosophila, mouse, and human. Currently, there are at least 14 known caspase genes, named caspase-1 through caspase-14. Table 1 recites ten human caspases along with their alternative names.

TABLE 1

| Caspase | Alternative name |
|---|---|
| Caspase-1 | ICE |
| Caspase-2 | ICH-1 |
| Caspase-3 | CPP32, Yama, apopain |
| Caspase-4 | ICEᵣₑₗII; TX, ICH-2 |
| Caspase-5 | ICEᵣₑₗII; TY |
| Caspase-6 | Mch2 |
| Caspase-7 | Mch3, ICE-LAP3, CMH-1 |
| Caspase-8 | FLICE; MACH; Mch5 |
| Caspase-9 | ICE-LAP6; Mch6 |
| Caspase-10 | Mch4, FLICE-2 |

Within the context of this invention, it should be understood that a caspase includes wild-type protein sequences, as well as other variants (including alleles) of the native protein sequence. Briefly, such variants may result from natural polymorphisms or may be synthesized by recombinant methodology, and differ from wild-type protein by one or more amino acid substitutions, insertions, deletions, or the like. Typically, when engineered, amino acid substitutions will be conservative, i.e., substitution of amino acids within groups of polar, non-polar, aromatic, charged, etc. amino acids. In the region of homology to the native sequence, variants should preferably have at least 90% amino acid sequence identity, and within certain embodiments, greater than 92%, 95%, or 97% identity. Such amino acid sequence identity may be determined by standard methodologies, including use of the National Center for Biotechnology Information BLAST search methodology available at www.ncbi.nlm.nih.gov. The identity methodologies preferred are those described in U.S. Pat. No. 5,691,179 and Altschul et al., Nucleic Acids Res. 25:3389-3402, 1997 all of which are incorporated herein by reference. If Gapped BLAST 2.0 is utilized, then it is utilized with default settings.

As will be appreciated by those skilled in the art, a nucleotide sequence encoding a caspase or variant may differ from the known native sequences, due to codon degeneracies, nucleotide polymorphisms, or amino acid differences. In other embodiments, variants should preferably hybridize to the native nucleotide sequence at conditions of normal stringency, which is approximately 25-30° C. below Tm of the native duplex (e.g., 5× SSPE, 0.5% SDS, 5× Denhardt's solution, 50% formamide, at 42° C. or equivalent conditions; see generally, Sambrook et al. Molecular Cloning: A Laboratory Manual, 2nd ed., Cold Spring Harbor Press, 1987; Ausubel et al., Current Protocols in Molecular Biology, Greene Publishing, 1987).

An “isolated nucleic acid molecule” refers to a polynucleotide molecule in the form of a separate fragment or as a component of a larger nucleic acid construct, that has been separated from its source cell (including the chromosome it normally resides in) at least once in a substantially pure form. Nucleic acid molecules may be comprised of a wide variety of nucleotides, including DNA, RNA, nucleotide analogues, or some combination of these.

A “stimulator of apoptosis” or “pro-apoptotic agent”, as used herein refers to an agent that increases the specific apoptotic activity of a cell. Illustrative examples of such stimulus are deprivation of a growth factor, Fas ligand, anti-Fas antibody, staurosporine, ultraviolet irradiation, gamma irradiation, tumor necrosis factor, and others well known in the art. Accordingly, a stimulator of apoptosis is an agent that increases the molecular activity of caspase molecules either directly or indirectly. In addition, a stimulator of apoptosis can be a polypeptide that is capable of increasing or inducing the apoptotic activity of a cell. Such polypeptides include those that directly regulate the apoptotic pathway such as Bax, Bad, Bcl-xS, Bak, Bik, and active caspases as well as those that indirectly regulate the pathway.

An “inhibitor of apoptosis” or “anti-apoptotic agent”, as used herein refers to an agent that decreases the apoptotic activity of a cell when compared to control agents. Illustrative examples of such anti-apoptotic agents include small molecules, fmk, p35, crmA, Bcl-2, Bcl-X_{L}, Mcl-1, E1B-19K from adenovirus, as well as antagonists of pro-apoptotic agents (e.g., antisense, ribozymes, antibodies, etc.). Accordingly, an inhibitor of apoptosis is an agent that decreases the molecular activity of caspase molecules either directly or indirectly.

An “apoptotic pathway protein”, as used herein refers to a protein involved in the cell death pathway. Illustrative examples include Bcl-2, Bcl-Xₛ, Bcl-X_{L}, Bik, Bak, Bax, Bad, caspase molecules, Apaf-1, cytochrome c, and the like.

As noted above, the present invention provides methods for the inhibition of programmed cell death, or apoptosis, by inhibition of members of the ICE/CED-3 family that facilitate antigen preservation by maintaining cellular integrity of the target tissue population. This would include not only inhibitors of ICE/CED-3 enzymatic activity, but also any method which specifically prevents the expression of ICE/CED-3 family encoding genes. Thus, antisense RNA or DNA comprised of nucleotide sequences complementary to ICE/CED-3 family member genes and capable of inhibiting the transcription or translation of the relevant proteins, expression of dominant negative forms of the ICE/CED-3 proteases (e.g., mutants engineered to replace the active site cysteine with another amino acid, like serine or alanine), or antibodies which bind to ICE/CED-3 family polypeptides, are within the scope of the invention, as are small molecule inhibitors, including peptides.

Before describing the methods of the invention, exemplary compounds useful in the methods of the invention are described below:

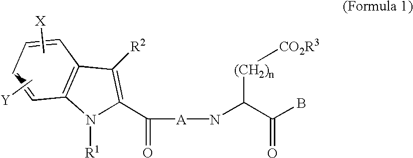

wherein:

n is 1 or 2;

R1 is alkyl, cycloalkyl, (cycloalkyl)alkyl, phenyl, (substituted)phenyl, phenylalkyl, (substituted)phenylalkyl, heteroaryl, (heteroaryl)alkyl or (CH₂)ₘCO₂R⁴,

wherein

m=1-4, and R⁴ is as defined below;

R² is a hydrogen atom, chloro, alkyl, cycloalkyl,(cycloalkyl)alkyl, phenyl, (substituted)phenyl, phenylalkyl, (substituted)phenylalkyl, heteroaryl, (heteroaryl)alkyl or (CH₂)ₚCO₂R⁵, wherein p=0-4, and R⁵ is as defined below;

R³ is a hydrogen atom, alkyl, cycloalkyl, (cycloalkyl)alkyl, phenylalkyl, or (substituted)phenylalkyl;

R⁴ is a hydrogen atom, alkyl, cycloalkyl, (cycloalkyl)alkyl, phenylalkyl, or (substituted)phenylalkyl;

R⁵ is a hydrogen atom, alkyl, cycloalkyl, (cycloalkyl)alkyl, phenylalkyl, or (substituted)phenylalkyl;

A is a natural or unnatural amino acid;

B is a hydrogen atom, a deuterium atom alkyl, cycloalkyl, (cycloalkyl)alkyl, phenyl, (substituted)phenyl, phenylalkyl, (substituted)phenylalkyl, heteroaryl, (heteroaryl)alkyl, halomethyl, CH₂ZR⁶, CH₂OCO(aryl), or CH₂OCO(heteroaryl), or CH₂OPO(R⁷)R⁸, where Z is an oxygen or a sulfur atom;

R⁶ is phenyl, substituted phenyl, phenylalkyl,(substituted phenyl)alkyl, heteroaryl or (heteroaryl)alkyl; and

R⁷ and R⁸ are independently selected from a group consisting of alkyl, cycloalkyl, phenyl, substituted phenyl, phenylalkyl,(substituted phenyl) alkyl and (cycloalkyl) alkyl; and

X and Y are independently selected from the group consisting of a hydrogen atom, halo, trihalomethyl, amino, protected amino, an amino salt, mono-substituted amino, di-substituted amino, carboxy, protected carboxy, a carboxylate salt, hydroxy, protected hydroxy, a salt of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl; or a pharmaceutically acceptable salt thereof.

As used in the above formula I and in formula 3 below, the term “alkyl” means a straight or branched C₁ to C₈ carbon chain such as methyl, ethyl, tent-butyl, iso-propyl, n-octyl, and the like.

The term “cycloalkyl” means a mono-, bi-, or tricyclic ring that is either fully saturated or partially unsaturated. Examples of such a ring include cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted with one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more, and preferably one or two, moieties chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, N—((C₁ to C₆ alkyl)sulfonyl)amino, N-(phenylsulfonyl)amino or by a substituted or unsubstituted phenyl group, such that in the latter case a biphenyl or naphthyl group results.

Examples of the term “substituted phenyl” includes a mono- or di(halo)phenyl group such as 2-, 3- or 4-chlorophenyl, 2,6-dichlorophenyl, 2,5-dichlorophenyl, 3,4-dichlorophenyl, 2-,3- or 4-bromophenyl, 3,4-dibromophenyl, 3-chloro-4-fluorophenyl, 2-, 3- or 4-fluorophenyl and the like; a mono or di(hydroxy)phenyl group such as 2-, 3-, or 4-hydroxyphenyl, 2,4-dihydroxyphenyl, the protected-hydroxy derivatives thereof and the like; a nitrophenyl group such as 2-, 3-, or 4-nitrophenyl; a cyanophenyl group, for example, 2-, 3- or 4-cyanophenyl; a mono- or di(alkyl)phenyl group such as 2-, 3- or 4-methylphenyl, 2,4-dimethylphenyl, 2-, 3- or 4-(iso-propyl)phenyl, 2-, 3-, or 4-ethylphenyl, 2-, 3- or 4-(n-propyl)phenyl and the like; a mono or di(alkoxy)phenyl group, for example, 2,6-dimethoxyphenyl, 2-, 3- or 4-(iso-propoxy)phenyl, 2-, 3- or 4-(t-butoxy)phenyl, 3-ethoxy-4-methoxyphenyl and the like; 2-, 3- or 4-trifluoromethylphenyl; a mono- or dicarboxyphenyl or (protected carboxy)phenyl group such as 2-, 3- or 4-carboxyphenyl or 2,4-di(protected carboxy)phenyl; a mono- or di(hydroxymethyl)phenyl or (protected hydroxymethyl)phenyl such as 2-, 3- or 4-(protected hydroxymethyl)phenyl or 3,4-di(hydroxymethyl)phenyl; a mono- or di(aminomethyl)phenyl or (protected aminomethyl)phenyl such as 2-, 3- or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like.

The term “(substituted phenyl)alkyl” means one of the above substituted phenyl groups attached to one of the above-described alkyl groups. Examples of such groups include 2-phenyl-1-chloroethyl, 2-(4′-methoxyphenyl)ethyl, 4-(2′,6′-dihydroxy phenyl)ₙ-hexyl, 2-(5′-cyano-3′-methoxyphenyl)ₙ-pentyl, 3-(2′,6′-dimethylphenyl)ₙ-propyl, 4-chloro-3-aminobenzyl, 6-(4′-methoxyphenyl)-3-carboxy(n-hexyl), 5-(4′-aminomethylphenyl)-3-(aminomethyl)ₙ-pentyl, 5-phenyl-3-oxo-n-pent-1-yl, (4-hydroxynapth-2-yl)methyl, and the like.

The terms “halo” and “halogen” refer to the fluoro, chloro, bromo or iodo groups. There can be one or more halogen, which are the same or different. Preferred halogens are chloro and fluoro.

The term “aryl” refers to aromatic five and six membered carbocyclic rings. Six membered rings are preferred.

The term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms. These five-membered or six-membered rings are fully unsaturated.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzothiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl groups. Substituents for the heteroaryl group are as heretofore defined, or as set forth below. As used in conjunction with the above substituents for heteroaryl rings, “trihalomethyl” can be trifluoromethyl, trichloromethyl, tribromomethyl or triiodomethyl, “lower alkoxy” means a C₁ to C₄ alkoxy group, similarly, “lower alkylthio” means a C₁ to C₄ alkylthio group. The term “substituted alkyl” means the above-defined alkyl group substituted from one to three times by a hydroxy, protected hydroxy, amino, protected amino, cyano, halo, trifluoromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined above substituted with the same groups as listed for a “substituted alkyl” group. The term “(monosubstituted)amino” refers to an amino group with one substituent chosen from the group consisting of phenyl, substituted phenyl, alkyl, substituted alkyl, C₁ to C₇ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted)amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different. The term “heteroaryl(alkyl)” denotes an alkyl group as defined above, substituted at any position by a heteroaryl group, as above defined.

Furthermore, the above optionally substituted five-membered or six-membered heterocyclic rings can optionally be fused to a aromatic 5-membered or 6-membered aryl or heteroaryl ring system. For example, the rings can be optionally fused to an aromatic 5-membered or 6-membered ring system such as a pyridine or a triazole system, and preferably to a benzene ring.

The term “pharmaceutically-acceptable salt” encompasses those salts that form with the carboxylate anions and includes salts formed with the organic and inorganic cations such as those chosen from the alkali and alkaline earth metals, (for example, lithium, sodium, potassium, magnesium, barium and calcium); and ammonium ion; and the organic cations (for example, dibenzylammonium, benzylammonium, 2-hydroxyethylammonium, bis(2-hydroxyethyl)ammonium, phenylethylbenzylammonium, dibenzylethylenediammonium, and like cations.) Other cations encompassed by the above term include the protonated form of procaine, quinine and N-methylglucosamine, the protonated forms of basic amino acids such as glycine, ornithine, histidine, phenylglycine, lysine, and arginine. Furthermore, any zwitterionic form of the instant compounds formed by a carboxylic acid and an amino group is referred to by this term. A preferred cation for the carboxylate anion is the sodium cation. Furthermore, the term includes salts that form by standard acid-base reactions with basic groups (such as amino groups) and includes organic or inorganic acids. Such acids include hydrochloric, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, cinnamic, and the like acids.

The compounds of Formula I may also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

The term “carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include t-butyl, 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxytrityl, 4,4′,4″-trimethoxytrityl, 2-phenylpropyl, trimethylsilyl, t-butyldimethylsilyl, phenacyl, 2,2,2-trichloroethyl, β-(trimethylsilyl)ethyl, P-(di(n-butyl)methylsilyl)ethyl, p-toluenesulfonylethyl, 4-nitrobenzylsulfonylethyl, allyl, cinnamyl, 1-(trimethylsilylmethyl)-propenyl and like moieties. The species of carboxy-protecting group employed is not critical so long as the derivatized carboxylic acid is stable to the conditions of subsequent reaction(s) and can be removed at the appropriate point without disrupting the remainder of the molecule. Further examples of these groups are found in C. B. Reese and E. Haslam, “Protective Groups in Organic Chemistry,” J. G. W. McOmie, Ed., Plenum Press, New York, N.Y., 1973, Chapter 5, respectively, and T. W. Greene and P. G. M. Wuts, “Protective Groups in Organic Synthesis,” 2nd ed., John Wiley and Sons, New York, N.Y., 1991, Chapter 5, each of which is incorporated herein by reference. A related term is “protected carboxy,” which refers to a carboxy group substituted with one of the above carboxy-protecting groups.

The term “hydroxy-protecting group” refers to readily cleavable groups bonded to hydroxyl groups, such as the tetrahydropyranyl, 2-methoxyprop-2-yl, 1-ethoxyeth-1-yl, methoxymethyl, β-methoxyethoxymethyl, methylthiomethyl, t-butyl, t-amyl, trityl, 4-methoxytrityl, 4,4′-dimethoxytrityl, 4,41,4″-trimethoxytrityl, benzyl, allyl, trimethylsilyl, (t-butyl)dimethylsilyl, 2,2,2-trichloroethoxycarbonyl, and the like.

Further examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W Greene and P. G. M. Wuts, “Protective Groups in Organic Synthesis,” Second Edition, John Wiley and Sons, New York; NY, 1991, Chapters 2 and 3. A preferred hydroxy-protecting group is the tert-butyl group. The related term “protected hydroxy” denotes a hydroxy group bonded to one of the above hydroxy-protecting groups.

The term “amino-protecting group” as used herein refers to substituents of the amino group commonly employed to block or protect the amino functionality while reacting other functional groups of the molecule. The term “protected (monosubstituted)amino” means there is an amino-protecting group on the monosubstituted amino nitrogen atom.

Examples of such amino-protecting groups include the formyl (“For”) group, the trityl group, the phthalimido group, the trichloroacetyl group, the trifluoroacetyl group, the chloroacetyl, bromoacetyl, and iodoacetyl groups, urethane-type protecting groups, such as t-butoxycarbonyl (“Boc”), 2-(4-biphenylyl)propyl-2-oxycarbonyl (“Bpoc”), 2-phenylpropyl-2-oxycarbonyl (“Poc”), 2-(4-xenyl)isopropoxycarbonyl, 1,1-diphenylethyl-1-oxycarbonyl, 1,1-diphenylpropyl-1-oxycarbonyl, 2-(3,5-dimethoxyphenyl)propyl-2-oxycarbonyl (“Ddz”), 2-(p-toluyl)propyl-2-oxycarbonyl, cyclopentanyloxycarbonyl, 1-methylcyclopentanyl-oxycarbonyl, cyclohexanyloxy-carbonyl, 1-methylcyclohexanyloxycarbonyl, 2-methylcyclohexanyl-oxycarbonyl, 2-(4-toluylsulfonyl)ethoxycarbonyl, 2-(methylsulfonyl)ethoxycarbonyl, 2-(triphenylphosphino)ethoxycarbonyl, 9-fluorenylmethoxycarbonyl (“Fmoc”), 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyl-oxycarbonyl, 2,2,2-trichloroethoxycarbonyl, 2-ethynyl-2-propoxycarbonyl, cyclopropylmethoxycarbonyl, isobornyloxycarbonyl, 1-piperidyloxycarbonyl, benzyloxycarbonyl (“Cbz”), 4-phenylbenzyloxycarbonyl, 2-methylbenzyloxycarbonyl, α-2,4,5,-tetramethylbenzyloxycarbonyl (“Tmz”), 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 4-(decyloxy)benzyloxycarbonyl and the like; the benzoylmethylsulfonyl group, the 2,2,5,7,8-pentamethylchroman-6-sulfonyl group (“PMC”), the dithiasuccinoyl (“Dts”) group, the 2-(nitro)phenyl-sulfenyl group (“Nps”), the diphenylphosphine oxide group, and like amino-protecting groups. The species of amino-protecting group employed is not critical so long as the derivatized amino group is stable to the conditions of the subsequent reaction(s) and can be removed at the appropriate point without disrupting the remainder of the molecule. Preferred amino-protecting groups are Boc, Cbz and Fmoc. Further examples of amino-protecting groups embraced by the above term are well known in organic synthesis and the peptide art and are described by, for example, T. W. Greene and P. G. M. Wuts, “Protective Groups in Organic Synthesis,” 2nd ed., John Wiley and Sons, New York, N.Y., 1991, Chapter 7, M. Bodanzsky, “Principles of Peptide Synthesis,” 1st and 2nd revised Ed., Springer-Verlag, New York, N.Y., 1984 and 1993, and J. M. Stewart and J. D. Young, “Solid Phase Peptide Synthesis,” 2nd Ed., Pierce Chemical Co., Rockford, Ill., 1984, E. Atherton and R. C. Shephard, “Solid Phase Peptide Synthesis —A Practical Approach” IRL Press, Oxford, England (1989), each of which is incorporated herein by reference. The related term “protected amino” defines an amino group substituted with an amino-protecting group discussed above.

The terms “natural and unnatural amino acid” refers to both the naturally occurring amino acids and other non-proteinogenic α-amino acids commonly utilized by those in the peptide chemistry arts when preparing synthetic analogues of naturally occurring peptides, including D and L forms. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine γ-carboxyglutamic acid, arginine, ornithine and lysine. Examples of unnatural alpha-amino acids include hydroxylysine, citrulline, kynurenine, (4-aminophenyl)alanine, 3-(2′-naphthyl)alanine, 3-(1′-naphthyl)alanine, methionine sulfone, (t-butyl)alanine, (t-butyl)glycine, 4-hydroxyphenyl-glycine, aminoalanine, phenylglycine, vinylalanine, propargyl-gylcine, 1,2,4-triazolo-3-alanine, thyronine, 6-hydroxytryptophan, 5-hydroxytryptophan, 3-hydroxy-kynurenine, 3-aminotyrosine, trifluoromethylalanine, 2-thienylalanine, (2-(4-pyridyl)ethyl)cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyro sine, norleucine, norvaline, alloisoleucine, homoarginine, thioproline, dehydro-proline, hydroxyproline, homoproline, indoline-2-carboxylic acid, 1,2,3,4-tetrahydroisoquinoline-3-carboxylic acid, 1,2,3,4-tetrahydroquinoline-2-carboxylic acid, α-amino-n-butyric acid, cyclohexylalanine, 2-amino-3-phenylbutyric acid, phenylalanine substituted at the ortho, meta, or para position of the phenyl moiety with one or two of the following groups: a (C₁ to C₄)alkyl, a (C₁ to C₄)alkoxy, a halogen or a nitro group, or substituted once with a methylenedioxy group; -2- and 3-thienylalanine; β-2- and 3-furanylalanine; β-2-, 3- and 4-pyridylalanine; β-(benzothienyl-2- and 3-yl)alanine; β-(1- and 2-naphthyl)alanine; O-alkylated derivatives of serine, threonine or tyrosine; S-alkylated cysteine, S-alkylated homocysteine, the O-sulfate, O-phosphate and O-carboxylate esters of tyrosine; 3-(sulfo)tyrosine, 3-(carboxy)tyrosine, 3-(phospho)tyrosine, the 4-methane-sulfonic acid ester of tyrosine, 4-methanephosphonic acid ester of tyrosine, 3,5-diiodotyrosine, 3-nitrotyrosine, ∈-alkyllysine, and delta-alkyl ornithine. Any of these α-amino acids may be substituted with a methyl group at the alpha position, a halogen at any position of the aromatic residue on the α-amino side chain, or an appropriate protective group at the O, N, or S atoms of the side chain residues. Appropriate protective groups are discussed above.

Depending on the choice of solvent and other conditions known to the practitioner skilled in the art, compounds of this invention may also take the ketal or acetal form, which forms are included in the instant invention.

In addition, it should be understood that the equilibrium forms of the compounds of this invention may include tautomeric forms. All such forms of these compounds are expressly included in the present invention.

The compounds useful in the methods of the invention may be modified by appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of exertion. In addition, the compounds may be altered to pro-drug form such that the desired compound is created in the body of the patient as the result of the action of metabolic or other biochemical processes on the pro-drug. Some examples of pro-drug forms include ketal, acetal, oxime, and hydrazone forms of compounds which contain ketone or aldehyde groups, especially where they occur in the group denoted as “A” in Formula I or the modified aspartic or glutamic residues attached to the group denoted as “A”.

In the above Formula I or in Formula 3 below, a group of optimal compounds occurs when n is one, more so when B is a hydrogen atom, and especially so when R³ is a hydrogen atom or a t-butyl group. Of note within this group of compounds as those when A is naturally-occurring amino acid. This latter group of compounds will be referred to herein as the “4-oxobutanoic compounds.”

Within this group of 4-oxobutanoic compounds is a group of optimal compounds wherein R¹ is a methyl group, that is, the N-methylindole compounds. One embodiment of this group of N-methylindole compounds occurs when A is an alanine, valine, leucine, phenylalanine, glycine or a proline residue. Compounds of note within each one of these groups of natural amino acid, N-methylindole compounds occur when the N-methylindole is otherwise unsubstituted, that is, wherein X, Y and R² are each a hydrogen atom, and optimally so when R³ is a hydrogen atom.

Another optimal group of 4-oxobutanoic compounds consists of the N-benzylindole compounds. For example, one group of the N-benzylindole compounds occurs when A is an alanine residue. Of note within this group of alanine compounds are those in which X, Y and R² are each a hydrogen atom, and especially so where R³ is a hydrogen atom.

An alternate optimal group of 4-oxobutanoic compounds occurs when the N-substituent of the indole group is a 1-butenyl group. An embodiment of this group of N-(1-butenyl)indole compounds occurs when A is a valine residue, and especially so when X, Y and R are each a hydrogen atom. An optimal group of this latter group of compounds occurs when R³ is a hydrogen atom.

Yet another group of optimal 4-oxobutanoic compounds occurs when the N-substituent of the indole ring is a 2′-acetic acid residue. An exemplary group of the N-(2′-acetic acid compounds) occurs when A is an alanine residue. An embodiment of this particular group of alanine compounds occurs when X, Y and R² are each a hydrogen atom, and especially so when R³ is a hydrogen atom.

A group of the 4-oxobutanoic compounds when the indole group is substituted on the nitrogen with 3′-propionic acid residue is another example of this invention. An optimal group of such N-(propionic acid)indole compounds occurs when A is an alanine residue. Of note within this group of alanine compounds are those when X, Y and R² are each a hydrogen atom, and especially so when R³ is a hydrogen atom.

Another optimal group of compounds of Formula I occurs wherein n is one and more so when B is a monofluoromethyl group. An embodiment of these monofluoromethyl compounds occurs when R³ is a hydrogen atom or a t-butyl group, and an even more so when A is a natural amino acid. An example of these compounds wherein A is a natural amino acid occurs when A is a valine residue. This latter group of valine compounds will be referred to herein as the “4-oxo-5-(fluoropentanoic acid) compounds.”

One optimal group of 4-oxo-5-(fluoropentanoic acid) compounds occurs when R¹ is a methyl group, in other words, the N-methylindole compounds. An exemplary group of such N-methylindole compounds occurs when R² is a methyl group and X and Y are each a hydrogen atom, and especially so when R³ is a hydrogen atom. Another exemplary group of such N-methylindole compounds occurs when R² is a chloro atom and X and Y are each a hydrogen atom, and especially so when R³ is a hydrogen atom. A third exemplary group of N-methylindole compounds occurs when R² is a chloro group, X is a 5-fluoro group, and Y is a hydrogen atom, and especially so when R³ is a hydrogen atom.

Another optimal group of 4-oxo-5-(fluoro-pentanoic acid) compounds is composed of N-(3′-phenylprop-1-yl)indole compounds. A group of note within this latter class of compounds occurs when R², X and Y are each a hydrogen atom, and especially so when R³ is a hydrogen atom.

A third optimal group of 4-oxo-5-(fluoro-pentanoic acid) compounds has an N-(carboxymethyl or protected carboxymethyl)indole moiety. An embodiment of this group occurs wherein R², X and Y are each a hydrogen atom, and especially so wherein R³ is a hydrogen atom and the nitrogen atom of the indole ring is substituted with a carboxymethyl group.

Another optimal class of compounds of Formula 1 occurs when n is one and B is a (2,6-dichlorobenzyloxy)-methyl group and especially so when R³ is a hydrogen atom or a t-butyl group, and when A is a natural amino acid. An example of such a compound occurs when R¹ is a methyl group and especially so when R² is a methyl group.

The compounds of Formula I may be synthesized using conventional techniques as discussed below. Advantageously, these compounds are conveniently synthesized from readily available starting materials.

One synthetic route for synthesizing compounds is set forth in the following Scheme 1:

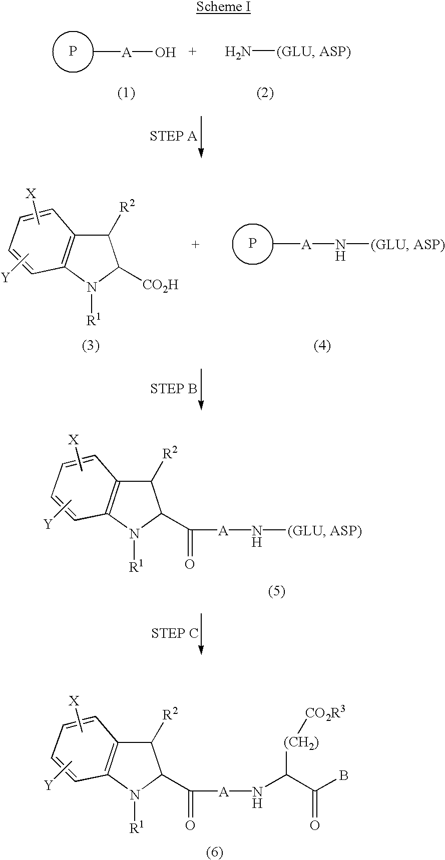

In the above Scheme I, Formula (2), that is H₂N-(Glu, Asp), is a modified aspartic or glutamic acid residue of Formulas 2a through 2d:

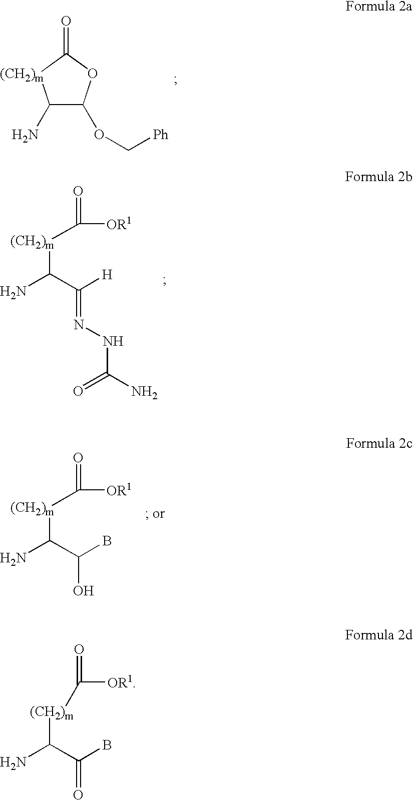

In the above Scheme I, \mathring{P} stands for an amino protecting group and (A) stands for a natural or unnatural amino acid, as discussed above.

The modified aspartic or glutamic acids of Formula 2a-d can be prepared by methods well known in the art. See, for example, European Patent Application 519,748; PCT Patent Application No. PCT/EP92/02472; PCT Patent Application No. PCT/US91/06595; PCT Patent Application No. PCT/US91/02339; European Patent Application No. 623,592; World Patent Application No. WO 93/09135; PCT Patent Application No. PCT/US94/08868; European Patent Application No. 623,606; European Patent Application No. 618,223; European Patent Application No. 533,226; European Patent Application No. 528,487; European Patent Application No. 618,233; PCT Patent Application No. PCT/EP92/02472; World Patent Application No. WO 93/09135; PCT Patent Application No. PCT/US93/03589; and PCT Patent Application No. PCT/US93/00481, all of which are herein incorporated by reference.

The coupling reactions carried out under Step A are performed in the presence of a standard peptide coupling agent such as the combination of the combination of dicyclohexylcarbodiimide(DCC) and 1-hydroxy-benzotriazole(HOBt), as well as the BOP (benzotriazolyloxy-trio-(dimethylamino)phosphonium hexafluorophosphate) reagent, pyBOP (benzotriazolyloxy-tris(N-pyrolidinyl)phosphoniumhexafluorophosphate), HBTU (O-benzotriazolyly-tetramethylisouronium-hexafluorophosphate), and EEDQ (1-ethyloxycarbonyl-2-ethyloxy-1,2-dihydroquinoline) reagents, the combination of 1-ethyl(3,3′-dimethyl-1′-aminopropyl)carbodiimide (EDAC) and HOBt, and the like, as discussed in J. Jones, “Amino Acid and Peptide Synthesis,” Steven G. Davis ed., Oxford University Press, Oxford, pp. 25-41 (1992); M. Bodanzky, “Principles of Peptide Synthesis,” Hafner et al. ed., Springer-Verlag, Berlin Heidelberg, pp. 9-52 and pp. 202-251 (1984); M. Bodanzky, “Peptide Chemistry, A Practical Textbook,” Springer-Verlag, Berlin Heidelberg, pp. 55-73 and pp. 129-180; and Stewart and Young, “Solid Phase Peptide Synthesis,” Pierce Chemical Company, (1984), all of which are herein incorporated by reference. The amino protecting group is then removed and the resulting amine is coupled to the 2-(carboxy)indole of (3) (Step B). Again, this coupling reaction uses the standard peptide coupling reactions mentioned above. The indole ring of (3) can be substituted before the reaction in Step B or afterwards. The synthesis and substitution reactions of such an indole ring is well known, as is described, for example, in Brown, R. T. and Joule, J. A. in “Heterocyclic chemistry (ed. P. G. Sammes) (Vol. 4 of Comprehensive Organic Chemistry, ed. D. Barton and W. D. Ollis), (1979), Pergamon Press, Oxford; Houlihan, W. J., (ed.) in “Indoles (The Chemistry of Heterocyclic Compounds [ed. A. Weissburger and E. C. Taylor], Vol. 25, Parts 1-3), Wiley Interscience, New York (1972); and Saxton, J. E. (ed.) in “Indoles (The Chemistry of Heterocyclic Compounds),” [ed. A. Weissburger and E. C. Taylor], Vol. 25, Part 4), Wiley Interscience, New York, (1979); all of which are incorporated herewith by reference.

In the case where the coupling reaction was carried out with the amino alcohol of Formula 2c, the alcohol moiety must be oxidized to the corresponding carbonyl compound prior to removal of the protecting groups. Preferred methods for the oxidation reaction include Swern oxidation (oxalyl chloride-dimethyl sulfoxide, methylene chloride at −78° C. followed by triethylamine); and Dess-Martin oxidation (Dess-Martin periodinane, t-butanol, and methylene chloride.) The protecting groups contained in substructures of the Formula 2a-d and A are removed by methods well known in the art. These reactions and removal of some or all of the protecting groups are involved in Step C in the above Scheme.

The compounds of Formula 3, below, are also useful in the methods of the invention:

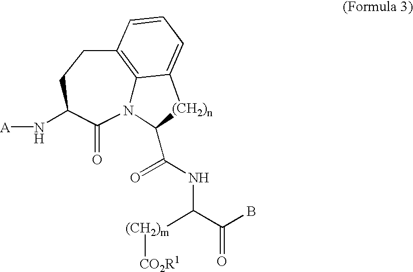

wherein:

n is 1 or 2;

m is 1 or 2;

A is R²CO—, R³—O—CO, or R⁴SO₂—;

a group of the formula:

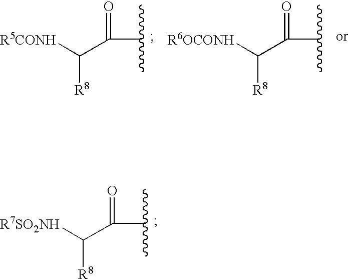

further wherein:

R¹ is a hydrogen atom, alkyl or phenylalkyl;

R² is alkyl, cycloalkyl, (cycloalkyl)alkyl, phenyl, phenylalkyl, substituted phenyl, (substituted phenyl)alkyl, heteroaryl, or (heteroaryl)alkyl;

R³ is alkyl, cycloalkyl, (cycloalkyl)alkyl, phenylalkyl, or (substituted phenyl)alkyl;

R⁴ is alkyl, cycloalkyl, (cycloalkyl)alkyl, phenyl, phenylalkyl, substituted phenyl, (substituted phenyl)alkyl, heteroaryl, or (heteroaryl)alkyl;

R⁵ is alkyl, cycloalkyl, (cycloalkyl)alkyl, phenyl, phenylalkyl, substituted phenyl, (substituted phenyl)alkyl, heteroaryl, or (heteroaryl)alkyl;

R⁶ is alkyl, cycloalkyl, (cycloalkyl)alkyl, phenylalkyl, or (substituted phenyl)alkyl;

R⁷ is alkyl, cycloalkyl, (cycloalkyl)alkyl, phenyl, phenylalkyl, substituted phenyl, (substituted phenyl)alkyl, heteroaryl, or (heteroaryl)alkyl;

R⁸ is an amino acid side chain chosen from the group consisting of natural and unnatural amino acids;

B is a hydrogen atom, a deuterium atom, alkyl, cycloalkyl, (cycloalkyl)alkyl, phenyl, phenylalkyl, (substituted)phenyl, (substituted)phenylalkyl, heteroaryl, (heteroaryl)alkyl, or halomethyl;

a group of the formula

—CH₂XR⁹;

wherein R⁹ is phenyl, phenylalkyl, substituted phenyl, (substituted phenyl)alkyl, heteroaryl, or (heteroaryl)alkyl; and X is an oxygen or a sulfur atom;

a group of the formula:

—CH₂—O—CO-(aryl);

a group of the formula:

—CH₂—O—CO-(heteroaryl);

a group of the formula:

—CH₂—O—PO(R¹⁰)R¹¹

wherein R¹⁰ and R¹¹ are independently selected from a group consisting of alkyl, cycloalkyl, phenyl, substituted phenyl, phenylalkyl and (substituted phenyl) alkyl; and the pharmaceutically-acceptable salts thereof.

The compounds of Formula 3 may also exist as solvates and hydrates. Thus, these compounds may crystallize with, for example, waters of hydration, or one, a number of, or any fraction thereof of molecules of the mother liquor solvent. The solvates and hydrates of such compounds are included within the scope of this invention.

The compounds of Formulas I and 3 of this invention may be synthesized using conventional techniques. Advantageously, these compounds are conveniently synthesized from readily available starting materials.

Thus, compounds of Formula 3 can be synthesized in general by combining a tricyclic nucleus set forth below in Formula 4:

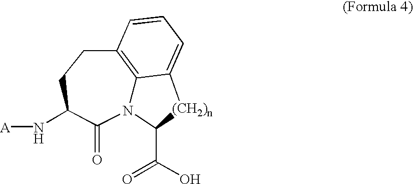

with the modified aspartic and glutamic acid residues of Formula 5 a-d:

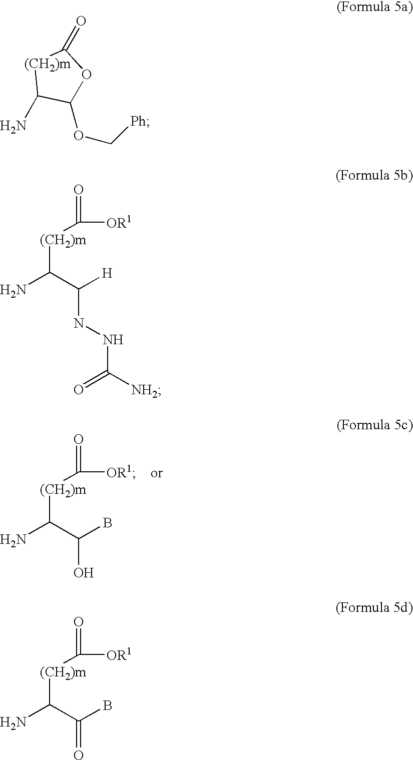

in the presence of a standard peptide coupling agents such as dicyclohexylcarbodiimide(DCC)-1-hydroxybenzotriazole(HOBt), BOP reagent, pyBOP, TBTU; EEDQ, 1-ethyl(3,3′-dimethyl-1′-aminopropyl)carbodiimide(EDAC)—HOBt, and the like, as discussed in J. Jones, “Amino Acid and Peptide Synthesis,” Steven G. Davis ed., Oxford University Press, Oxford, pp. 25-41 (1992), herein incorporated by reference. In the above formula, A is an amino protecting group. The amino protecting group is then removed and the resulting amine is combined with the substituted acyl group of Formula 6:

R^{C}—CO—X (6)

or the sulfonyl group of Formula 7:

R⁴SO₂—X. (7)

In the above formulas, R¹ is as defined above, and R^{c} is R², R³—O, R⁴, or any of the side chains containing R⁸ as defined for group A in Formula 3. Of course, such moieties would have any hydroxy, carboxy or amino groups in the protected form so as not to interfere with the coupling reaction (Formula 5a-d, the acylation reaction (Formula 4) or the sulfonation reaction (Formula 7). X in the above Formulas represents a facile leaving group for the acylation or sulfonation reactions.

In the case where the coupling reaction was carried out with the amino alcohol of Formula 5c, the alcohol moiety must be oxidized to the corresponding carbonyl compound prior to removal of the protecting groups. Preferred methods for the oxidation reaction include Swern oxidation (oxalyl chloride-dimethyl sulfoxide, methylene chloride at −78° C. followed by triethylmine; and Dess-Martin oxidation (Dess-Martin periodinane, t-butanol, and methylene chloride.) The protecting groups contained in substructures of the Formula 5a-d and A are removed by methods well known in the art.

The tricyclic nucleus of Formula 3 is synthesized by methods known in the art. For example, see D. S. Karanewsky, U.S. Pat. No. 5,504,080 issued Apr. 2, 1996; J. A. Robl et al., Tetrahedron Letters 36:1593-1596 (1995); and S. De Lombaert et al., Tetrahedron Letters. 35:7513-7516 (1994), all of which are incorporated herein by reference.

The modified aspartic or glutamic acid for Formula 5a-d can be elaborated by methods well known in the art. See, for example, European Patent Application 519,748; PCT Patent Application No. PCT/EP92/02472; PCT Patent Application No. PCT/US91/06595; PCT Patent Application No. PCT/US91/02339; European Patent Application No. 623,592; World Patent Application No. WO 93/09135; PCT Patent Application No. PCT/US94/08868; European Patent Application No. 623,606; European Patent Application No. 618,223; European Patent Application No. 533,226; European Patent Application No. 528,487; European Patent Application No. 618,233; PCT Patent Application No. PCT/EP92/02472; World Patent Application No. WO 93/09135; PCT Patent Application No. PCT/US93/03589; and PCT Patent Application No. PCT/US93/00481, all of which are herein incorporated by reference.

The acyl group of Formula 6 and the corresponding R⁴SO² groups are also synthesized by methods well known in the art. See, for example, U.S. Pat. No. 5,504,080, issued Apr. 2, 1996, herein incorporated by reference. While this group can be elaborated once bonded to the tricyclic nucleus, it is preferable that it be intact before being attached to the nucleus.

Once the side chains of Formula 5 and Formula 6 or Formula 7 are bonded to the tricyclic nucleus of Formula 3, one skilled in the art would usually remove any amino, hydroxy, or carboxy-protecting groups to enhance the activity of the synthesized molecule.

In another aspect of the present invention Formula 8 may be utilized:

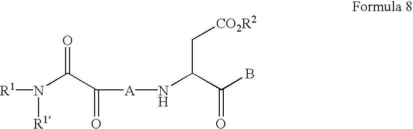

wherein:

A is a natural or unnatural amino acid of Formula IIa-i:

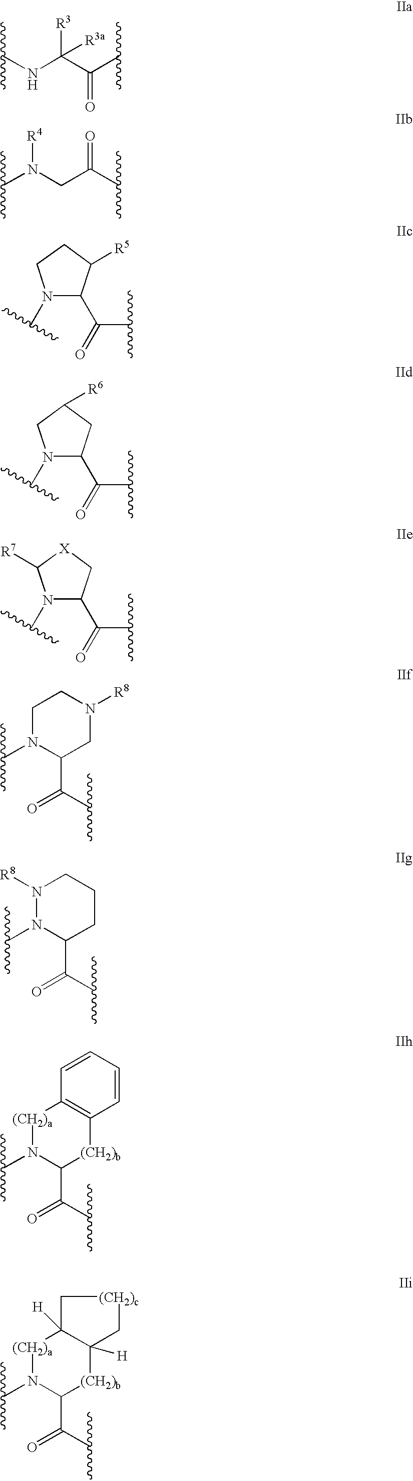

B is a hydrogen atom, a deuterium atom, alkyl, cycloalkyl, phenyl, substituted phenyl, naphthyl, substituted naphthyl, 2-benzoxazolyl, substituted 2-oxazolyl, (CH₂)ₙcycloalkyl, (CH₂)ₙphenyl, (CH₂)ₙ(substituted phenyl), (CH₂)ₙ(1 or 2-naphthyl), (CH₂)ₙ(substituted 1 or 2-naphthyl), (CH₂)ₙ(heteroaryl), (CH₂)ₙ(substituted heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c:

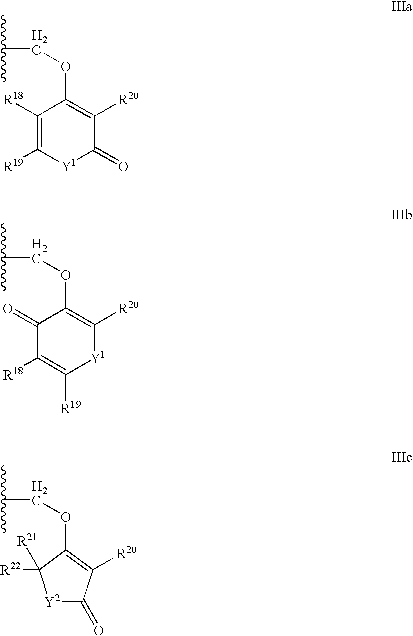

R¹ is alkyl, cycloalkyl, substituted cycloalkyl, (cycloalkyl)alkyl, substituted (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heterocycle, substituted heterocycle, (heterocycle)alkyl, substituted (heterocycle)alkyl, R^{1a}(R^{1b})N, or R^{1c}O;

R^{1′} is hydrogen, alkyl, phenyl, substituted phenyl, naphthyl, substituted naphthyl, heterocycle or substituted heterocycle;

or R¹ and R^{1′} taken together with the nitrogen atom to which they are attached form a heterocycle or substituted heterocycle;

R² is hydrogen, lower alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, or substituted (1 or 2 naphthyl)alkyl;

and wherein:

R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl, with the proviso that R^{1a} and R^{1b} cannot both be hydrogen;

R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl;

R³ is C₁₋₆ lower alkyl, cycloalkyl, phenyl, substituted phenyl, (CH₂)ₙNH₂, (CH₂)ₙNHCOR⁹, (CH₂)ₙN(C═NH)NH₂, (CH₂)ₘCO₂R², (CH₂)ₘOR¹⁰, (CH₂)ₘSR¹¹, (CH₂)ₙcycloalkyl, (CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;

R^{3a} is hydrogen or methyl, or R³ and R^{3a} taken together are —(CH₂)_{d}— where d is an integer from 2 to 6;

R⁴ is phenyl, substituted phenyl, (CH₂)ₘphenyl, (CH₂)ₘ(substituted phenyl), cycloalkyl, or benzofused cycloalkyl;

R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);

R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹ or NHCOR⁹;

R⁷ is hydrogen, oxo (i.e., =O), lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;

R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R¹⁴;

R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)_{b}(1 or 2-naphthyl);

R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);

R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);

R¹⁴ is hydrogen or lower alkyl;

or R¹³ and R¹⁴ taken together form a five to seven membered carbocyclic or heterocyclic ring, such as morpholine, or N-substituted piperazine;

R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);

R¹⁶ and R¹⁷ are independently lower alkyl, cycloalkyl, phenyl, substituted phenyl, naphthyl, phenylalkyl, substituted phenylalkyl, or (cycloalkyl)alkyl;

R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂—;

R²⁰ is hydrogen, alkyl, phenyl, substituted phenyl, (CH₂)ₙphenyl, (CH₂)ₙ(substituted phenyl);

R²¹, R²² and R²³ are independently hydrogen, or alkyl;

X is CH₂, (CH₂)₂, (CH₂)₃, or S;

Y¹ is O or NR²³;

Y² is CH₂, O, or NR^{23;}

a is 0 or 1;

b is 1 or 2, provided that when a is 1 then b is 1;

c is 1 or 2, provided that when c is 1 then a is 0 and b is 1;

m is 1 or 2; and

n is 1, 2, 3 or 4;

or a pharmaceutically acceptable salt thereof.

This compound and its synthesis are fully described in PCT Publication WO 00/01666 and related U.S. Patent Applications 09/177,549, which are incorporated by reference herein in their entirety.

A further compound that is useful in the context of the present invention is the compound of Formula 9:

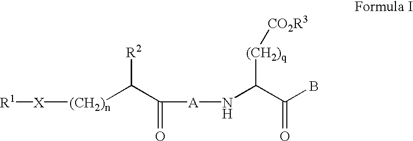

wherein:

n is 0, 1 or 2;

q is 1 or 2;

X is CH₂, C═O, O, S, NH, C═ONH or CH₂OC═ONH;

A is a natural or unnatural amino acid of Formula IIa-i:

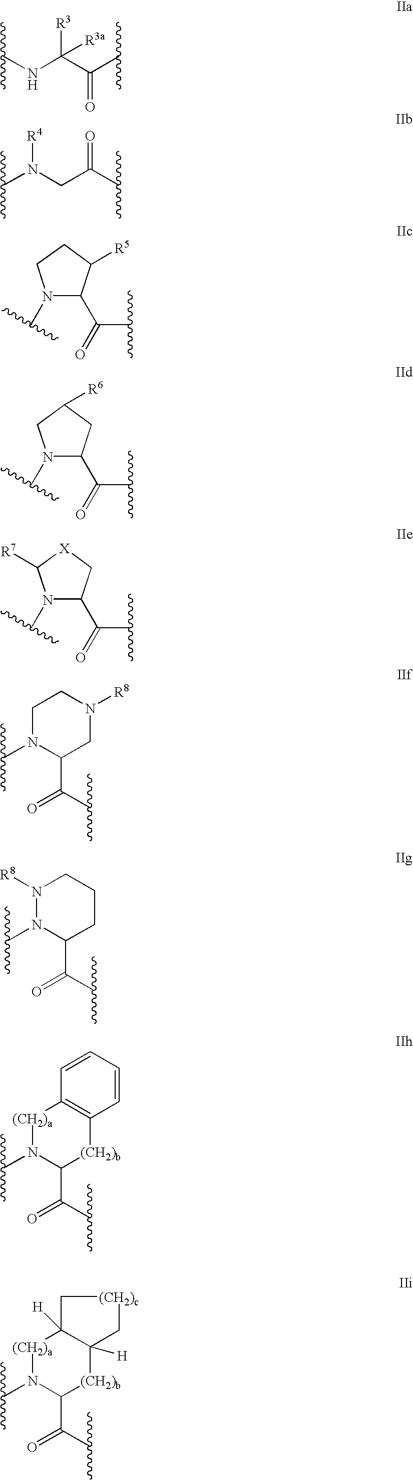

B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₘcycloalkyl, (CH₂)ₘphenyl, (CH₂)ₘ(substituted phenyl), (CH₂)ₘ(1 or 2-naphthyl), (CH₂)ₘheteroaryl, halomethyl, CO₂R¹³, CONR¹⁴R¹⁵, CH₂ZR¹⁶, CH₂OCO(aryl), CH₂OCO(substituted aryl), CH₂OCO(heteroaryl), CH₂OCO(substituted heteroaryl), or CH₂OPO(R¹⁷)R¹⁸, where Z is an oxygen or a sulfur atom, or B is a group of the Formula IIIa-c:

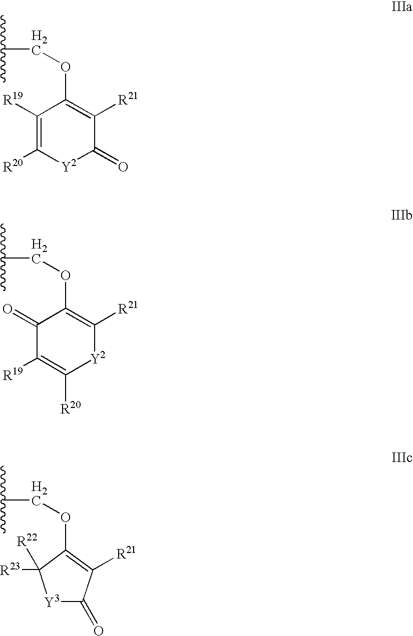

R¹ is phenyl, substituted phenyl, naphthyl, substituted naphthyl, heteroaryl, or substituted heteroaryl;

R² is hydrogen, alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) substituted or unsubstituted pyridyl, thienyl, furyl, thiazolyl, imidazolyl, pyrazolyl, isoxazolyl, pyrazinyl, pyrimidyl, triazinyl, tetrazolyl, and indolyl;

R³ is hydrogen, alkyl, cycloalkyl, (cycloalkyl)alkyl, phenylalkyl, or substituted phenylalkyl;

and wherein

R⁴ is alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) pyridyl, thienyl, furyl, thiazolyl, imidazolyl, pyrazolyl, isoxazolyl, pyrazinyl, pyrimidyl, triazinyl, tetrazolyl, and indolyl;

R^{4a} is hydrogen or methyl, or R⁴ and R^{4a} taken together are —(CH₂)_{d}— where d is an integer from 2 to 6;

R⁵ is phenyl, substituted phenyl, (CH₂)ₚphenyl, (CH₂)ₚ(substituted phenyl), cycloalkyl, or benzofused cycloalkyl;

R⁶ is hydrogen, alkyl, cycloalkyl, phenyl, substituted phenyl, (CH₂)ₘcycloalkyl, (CH₂)ₘphenyl, (CH₂)ₘ(substituted phenyl), or (CH₂)ₘ(1 or 2-naphthyl);

R⁷ is hydrogen, fluorine, oxo (i.e., =O), alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹¹, SR¹², or NHCOR¹⁰;

R⁸ is hydrogen, oxo, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R⁹ is alkyl, cycloalkyl, (CH₂)ₘcycloalkyl, (CH₂)ₘphenyl, (CH₂)ₘ(substituted phenyl), (CH₂)ₘ(1 or 2-naphthyl), or COR¹⁰;

R¹⁰ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹³, or NR¹⁴R¹⁵;

R¹¹ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹² is alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹³ is alkyl, cycloalkyl, (CH₂)ₘcycloalkyl, (CH₂)ₘphenyl, (CH₂)ₘ(substituted phenyl), or (CH₂)ₘ(1 or 2-naphthyl);

R¹⁴ is hydrogen, alkyl, cycloalkyl, phenyl, substituted phenyl, naphthyl, substituted naphthyl, (CH₂)ₘcycloalkyl, (CH₂)ₘphenyl, (CH₂)ₘ(substituted phenyl), or (CH₂)ₘ(1 or 2-naphthyl);

R¹⁵ is hydrogen or alkyl; or

R¹⁴ and R¹⁵ taken together form a five, six or seven membered carbocyclic or heterocyclic ring, such as morpholine or N-substituted piperazine;

R¹⁶ is phenyl, substituted phenyl, naphthyl, substituted naphthyl, heteroaryl, (CH₂)ₘphenyl, (CH₂)ₘ(substituted phenyl), (CH₂)ₘ(1 or 2-naphthyl), or (CH₂)ₘheteroaryl;

R¹⁷ and R¹⁸ are independently alkyl, cycloalkyl, phenyl, substituted phenyl, naphthyl, or phenylalkyl, substituted phenylalkyl, or (cycloalkyl)alkyl;

R¹⁹ and R²⁰ are independently hydrogen, alkyl, phenyl, substituted phenyl, (CH₂)ₘphenyl, or (CH₂)ₘ(substituted phenyl), or R¹⁹ and R²⁰ taken together are —(CH═CH)₂—;

R²¹ is hydrogen, alkyl, phenyl, substituted phenyl, (CH₂)ₘphenyl, (CH₂)ₘ(substituted phenyl);

R²², R²³ and R²⁴ are independently hydrogen or alkyl;

Y¹ is CH₂, (CH₂)₂, (CH₂)₃, or S;

Y² is O or NR²⁴;

Y³ is CH₂, O, or NR²⁴;

a is 0 or 1 and b is 1 or 2, provided that when a is 1 then b is 1;

c is 1 or 2, provided that when c is 1 then a is 0 and b is 1;

m is 1, 2, 3 or 4; and

p is 1 or 2;

or a pharmaceutically acceptable salt thereof.

This compound and its synthesis are fully described in PCT Publication WO 00/23421 and related U.S. Patent Applications 09/177,546, which are incorporated by reference herein in their entirety.

A further compound that may be used in the present invention comprises the compounds of the Formula 10:

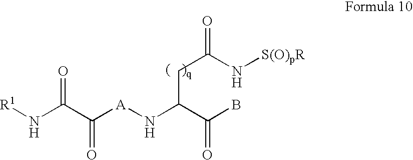

wherein:

p is 1 or 2;

q is 1 or 2;

R and R¹ are the same or different and independently alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, substituted (heteroaryl)alkyl, R^{1a}(R^{1b})N or R^{1c}O;

A is a natural or unnatural amino acid of Formula IIa-i:

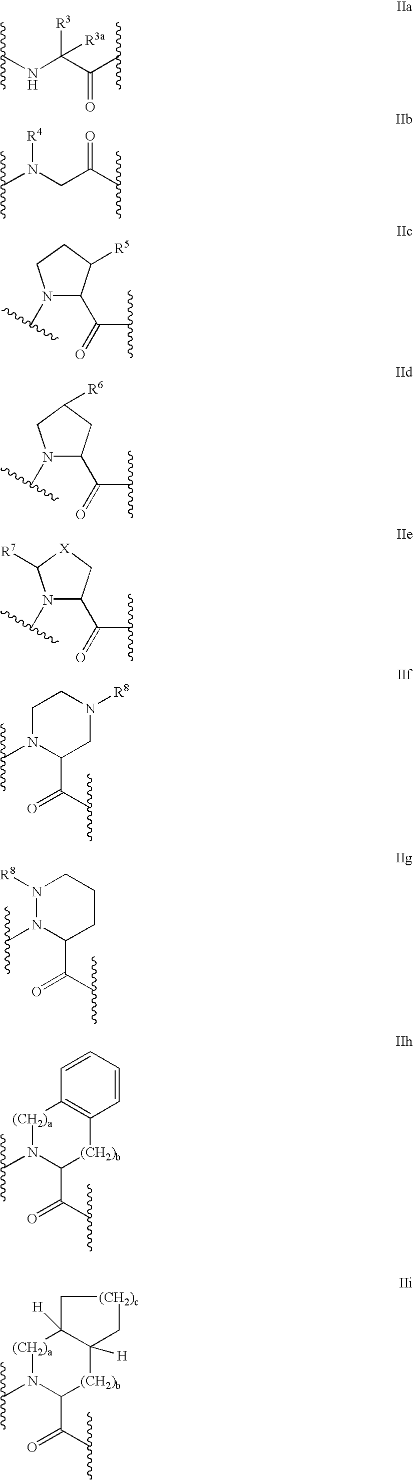

B is a hydrogen atom, a deuterium atom, alkyl, cycloalkyl, phenyl, substituted phenyl, naphthyl, substituted naphthyl, 2-benzoxazolyl, substituted 2-oxazolyl, (CH₂)ₙcycloalkyl, (CH₂)ₙphenyl, (CH₂)ₙ(substituted phenyl), (CH₂)ₙ(1 or 2-naphthyl), (CH₂)ₙ(substituted 1 or 2-naphthyl), (CH₂)ₙ(heteroaryl), (CH₂)ₙ(substituted heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c:

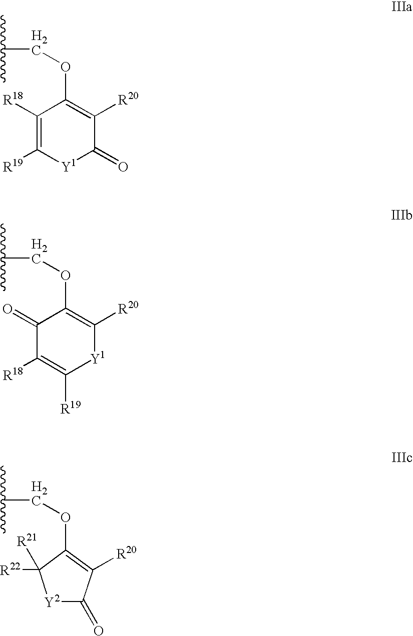

and wherein:

R^{1a} and R^{1b} are the same or different and, at each occurrence, independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl, with the proviso that R^{1a} and R^{1b} cannot both be hydrogen;

R^{1c} is, at each occurrence, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl;

R³ is C₁₋₆ lower alkyl, cycloalkyl, phenyl, substituted phenyl, (CH₂)ₙNH₂, (CH₂)ₙNHCOR⁹, (CH₂)ₙN(C═NH)NH₂, (CH₂)ₘCO₂R², (CH₂)ₘOR¹⁰, (CH₂)ₘSR¹¹, (CH₂)ₙcycloalkyl, (CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;

R^{3a} is hydrogen or methyl, or R³ and R^{3a} taken together are —(CH₂)_{d}— where d is an integer from 2 to 6;

R⁴ is phenyl, substituted phenyl, (CH₂)ₘphenyl, (CH₂)ₙ(substituted phenyl), cycloalkyl, or benzofused cycloalkyl;

R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);

R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹ or NHCOR⁹;

R⁷ is hydrogen, oxo (i.e., =O), lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;

R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R^{14;}

R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);

R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);

R¹⁴ is hydrogen or lower alkyl;

or R¹³ and R¹⁴ taken together form a five to seven membered carbocyclic or heterocyclic ring, such as morpholine, or N-substituted piperazine;

R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);

R¹⁶ and R¹⁷ are independently lower alkyl, cycloalkyl, phenyl, substituted phenyl, naphthyl, phenylalkyl, substituted phenylalkyl, or (cycloalkyl)alkyl;

R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂—;

R²⁰ is hydrogen, alkyl, phenyl, substituted phenyl, (CH₂)ₙphenyl, (CH₂)ₙ(substituted phenyl);

R²¹, R²² and R²³ are independently hydrogen, or alkyl;

X is CH₂, (CH₂)₂, (CH₂)₃, or S;

Y¹ is O or NR²³;

Y² is CH₂, O, or NR²³;

a is 0 or 1 and b is 1 or 2, provided that when a is 1 then b is 1;

c is 1 or 2, provided that when c is 1 then a is 0 and b is 1;

m is 1 or 2; and

n is 1,2, 3 or 4;

or a pharmaceutically acceptable salt thereof.

This compound and its synthesis are fully described in U.S. application Ser. No. 09/482,813, which is incorporated by reference herein in its entirety.

A further compound that may be used in the context of the present invention comprises the compounds of the Formula 11:

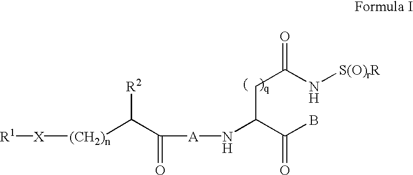

wherein:

n is 0, 1 or 2;

q is 1 or 2;

r is 1 or 2;

R is lower alkyl, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, substituted (heteroaryl)alkyl, NR^{a}(R^{b}) or OR^{c};

R¹ is phenyl, substituted phenyl, naphthyl, substituted naphthyl, heteroaryl, or substituted heteroaryl;

R² is hydrogen, alkyl, cycloalkyl, phenyl, substituted phenyl, (CH₂)ₚCO₂R³, (CH₂)ₘNH₂, (CH₂)ₘNHCOR¹⁰, (CH₂)ₘN(C═NH)NH₂, (CH₂)ₚOR¹¹, (CH₂)ₚSR², (CH₂)ₘcycloalkyl, (CH₂)ₘphenyl, (CH₂)ₘ(substituted phenyl), (CH₂)ₘ(l or 2-naphthyl), or (CH₂)ₘheteroaryl, wherein heteroaryl includes (but is not limited to) substituted or unsubstituted pyridyl, thienyl, furyl, thiazolyl, imidazolyl, pyrazolyl, isoxazolyl, pyrazinyl, pyrimidyl, triazinyl, tetrazolyl, and indolyl;

X is CH₂, C═O, O, S, NH, C═ONH or CH₂OC═ONH;

A is a natural or unnatural amino acid of Formula IIa-i:

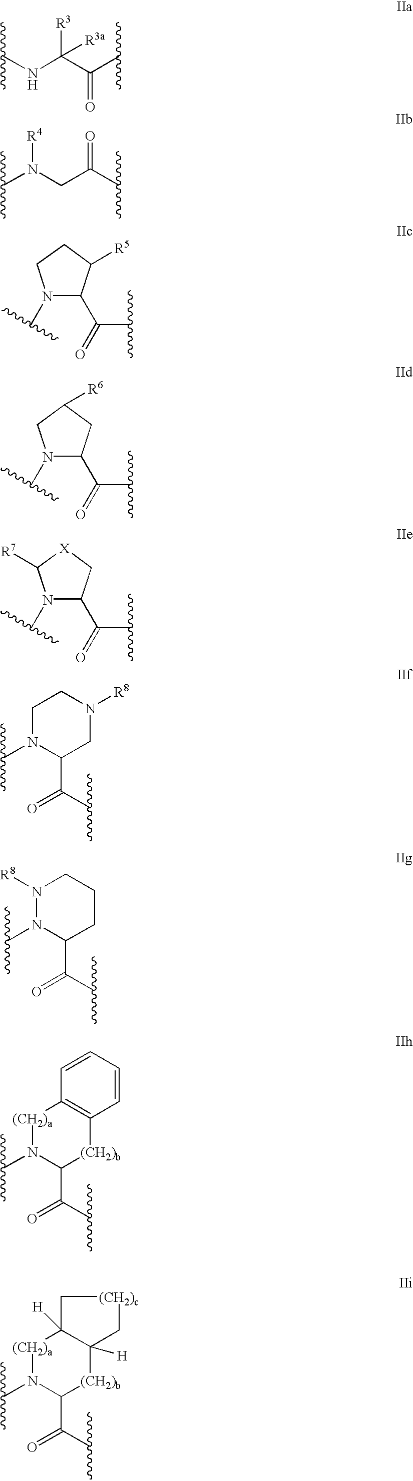

B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₘcycloalkyl, (CH₂)ₘphenyl, (CH₂)ₘ(substituted phenyl), (CH₂)ₘ(1 or 2-naphthyl), (CH₂)ₘheteroaryl, halomethyl, CO₂R¹³, CONR¹⁴R¹⁵, CH₂ZR¹⁶, CH₂OCO(aryl), CH₂OCO(substituted aryl), CH₂OCO(heteroaryl), CH₂OCO(substituted heteroaryl), or CH₂OPO(R¹⁷)R¹⁸, where Z is an oxygen or a sulfur atom, or B is a group of the Formula IIIa-c:

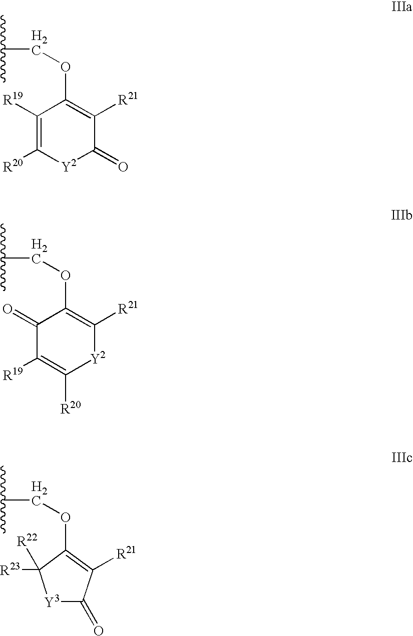

and wherein

R^{a} and R^{b} are the same or different and independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl, with the proviso that R^{a} and R^{b} cannot both be hydrogen;

R^{c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, substituted (1 or 2 naphthyl)alkyl, heteroaryl, substituted heteroaryl, (heteroaryl)alkyl, or substituted (heteroaryl)alkyl;

R³ is hydrogen, alkyl, cycloalkyl, (cycloalkyl)alkyl, phenylalkyl, or substituted phenylalkyl;

R⁴ is alkyl, cycloalkyl, phenyl, substituted phenyl, (CH₂)ₘNH₂, (CH₂)ₘNHCOR¹⁰, (CH₂)ₘN(C═NH)NH₂, (CH₂)ₚCO₂R³, (CH₂)ₚOR¹¹, (CH₂)ₚSR¹², (CH₂)ₘcycloalkyl, (CH₂)ₘphenyl, (CH₂)ₘ(substituted phenyl), (CH₂)ₘ(1 or 2-naphthyl), or (CH₂)ₘheteroaryl, wherein heteroaryl includes (but is not limited to) pyridyl, thienyl, furyl, thiazolyl, imidazolyl, pyrazolyl, isoxazolyl, pyrazinyl, pyrimidyl, triazinyl, tetrazolyl, and indolyl,

R⁴a is hydrogen or methyl, or

R⁴ and R^{4a} taken together are —(CH₂)_{d}— where d is an integer from 2 to 6;

R⁵ is phenyl, substituted phenyl, (CH₂)ₚphenyl, (CH₂)ₚ(substituted phenyl), cycloalkyl, or benzofused cycloalkyl;

R⁶ is hydrogen, alkyl, cycloalkyl, phenyl, substituted phenyl, (CH₂)ₘcycloalkyl, (CH₂)ₘphenyl, (CH₂)ₘ(substituted phenyl), or (CH₂)ₘ(1 or 2-naphthyl);

R⁷ is hydrogen, fluorine, oxo (i.e., =O), alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(l or 2-naphthyl), OR¹¹, SR¹², or NHCOR¹⁰;

R⁸ is hydrogen, oxo, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R⁹ is alkyl, cycloalkyl, (CH₂)ₘCycloalkyl, (CH₂)ₘphenyl, (CH₂)ₘ(substituted phenyl), (CH₂)ₘ(l or 2-naphthyl), or COR¹⁰;

R¹⁰ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), (CH₂)ₘ(1 or 2-naphthyl), OR¹³, or NR¹⁴R¹⁵;

R¹¹ is hydrogen, alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹² is alkyl, cycloalkyl, phenyl, substituted phenyl, naphthyl, (CH₂)ₘcycloalkyl, (CH₂)ₘphenyl, (CH₂)ₘ(substituted phenyl), or (CH₂)ₘ(1 or 2-naphthyl);

R¹³ is alkyl, cycloalkyl, (CH₂)ₘcycloalkyl, (CH₂)ₘphenyl, (CH₂)ₘ(substituted phenyl), or (CH₂)ₘ(1 or 2-naphthyl);

R¹⁴ is hydrogen, alkyl, cycloalkyl, phenyl, substituted phenyl, naphthyl, substituted naphthyl, (CH₂)ₘcycloalkyl, (CH₂)ₘphenyl, (CH₂)ₘ(substituted phenyl), or (CH₂)ₘ(1 or 2-naphthyl);

R¹⁵ is hydrogen or alkyl; or

R¹⁴ and R¹⁵ taken together form a five, six or seven membered carbocyclic or heterocyclic ring, such as morpholine or N-substituted piperazine;

R¹⁶ is phenyl, substituted phenyl, naphthyl, substituted naphthyl, heteroaryl, (CH₂)ₘphenyl, (CH₂)ₘ(substituted phenyl), (CH₂)ₘ(1 or 2-naphthyl), or (CH₂)ₘheteroaryl;

R¹⁷ and R¹⁸ are independently alkyl, cycloalkyl, phenyl, substituted phenyl, naphthyl, or phenylalkyl, substituted phenylalkyl, or (cycloalkyl)alkyl;

R¹⁹ and R²⁰ are independently hydrogen, alkyl, phenyl, substituted phenyl, (CH₂)ₘphenyl, or (CH₂)ₘ(substituted phenyl), or R¹⁹ and R²⁰ taken together are —(CH═CH)₂—;

R²¹ is hydrogen, alkyl, phenyl, substituted phenyl, (CH₂)ₘphenyl, (CH₂)ₘ(substituted phenyl);

R²², R²³, and R²⁴ are independently hydrogen or alkyl;

Y¹ is CH₂, (CH₂)₂, (CH₂)₃, or S;

Y² is 0 or NR²⁴;

Y³ is CH₂, O, or NR²⁴;

a is 0 or 1 and b is 1 or 2, provided that when a is 1 then b is 1;

c is 1 or 2, provided that when c is 1 then a is 0 and b is 1;

m is 1,2, 3 or 4; and

p is 1 or 2;

or a pharmaceutically acceptable salt thereof.

This compound and its synthesis are fully described in U.S. application Ser. No. 09/550,917, which is incorporated by reference herein in its entirety.

Methods for Inhibiting Apoptosis

The present invention provides methods for the inhibition of programmed cell death, or apoptosis, by inhibition of members of the ICE/CED-3 family. The invention provides new uses for not only inhibitors of ICE/CED-3 enzymatic activity, but also any method which specifically prevents the expression of ICE/CED-3 family encoding genes. Thus, antisense RNA or DNA comprised of nucleotide sequences complementary to ICE/CED-3 family member genes and capable of inhibiting the transcription or translation of the relevant proteins, expression of dominant negative forms of the ICE/CED-3 proteases (e.g., mutants engineered to replace the active site cysteine with another amino acid, like serine or alanine), or antibodies which bind to ICE/CED-3 family polypeptides, are within the scope of the invention, as are small molecule inhibitors, including peptides and especially the compounds presented herein.

In a first aspect, the invention provides a method for enhancing and/or preserving the antigenicity of bacterially or virally infected tissues by contacting the cells with an effective amount of a reagent which suppresses the activity of one or more ICE/CED-3 family members, inhibiting the programmed cell death of immature precursors and/or mature cells. In one embodiment, virally infected cells are contacted and preserved thus allowing for retention on the cell surface, viral antigens present thereon. Those of skill in the art would readily recognize that the present invention is applicable to any virus type, including without limitation, herpes, human immunodeficiency virus, cytomegalovirus, hepatitis, polio virus, and any virus for which a component thereof may be present on the surface of virally infected cells, thereby allowing detection.

The method includes contacting the desired cells with an inhibiting effective amount of a reagent which suppresses ICE/CED-3 activity. The term “contacting” as used herein means exposing the cells to the ICE/CED-3 family inhibitor(s) such that the inhibitor(s) can effectively inhibit ICE/CED-3 activity thereby inhibiting apoptosis in the cells and allowing the cells to proliferate and accumulate. The term “inhibiting effective amount” means that amount of ICE/CED-3 inhibitor that effectively blocks ICE/CED-3 enzymatic activity in intact target cells. It will be apparent that one or more ICE/CED-3 family inhibitors can be used simultaneously in the method of the invention. Examples of such reagents are commonly known in the art, including Cbz-ValAlaAsp-CH₂F, Cbz-ValAlaAsp-CH₂OCO (2,6-diCl-C₆H₄), Cbz-ValAlaAsp-CH₂F, methyl ester, Ac-AspValAlaAsp-CH₂F. Exemplary compounds include Formula I and Formula 3 as described supra.

Detection of ICE/CED-3 activity is by standard methods, such as an enzymatic assay to measure the fluorescence generated by enzymatic cleavage of aminomethylcoumarin (AMC) conjugated to a relevant peptide (e.g., Ac-DEVD-amc). Such assays are standard in the art (Armstrong et al., J. Biol. Chem.271:16850 (1996)); Fernandes-Alnemri et al. Cancer Res., 55:6045 (1995)). In addition, the inhibition of ICE (Caspase-1) activity can be measured by a bioassay for IL-1β. ICE/CED-3 activity is preferably suppressed by the ICE/CED-3 family inhibitor(s) by at least about 75%, and preferably by about 90%.

The “cells” or “cell population” includes a cell types including mammalian cells, such as precursor cells (e.g., pluripotent stem cells) and/or differentiated, mature cells.

The invention provides methods to preserve the viability of cells, such as neutrophils/granulocytes ex vivo for subsequent analysis by assisting in the maintenance of cellular integrity and thus antigen presentation.

The reagents of the present invention are “ICE/CED-3 inhibitors” in that they inhibit the catalytic activity of members of the ICE/CED-3 family in a reversible or an irreversible manner. The term “irreversible” as used herein means the formation of a covalent bond between the ICE/CED-3 family member and the inhibitor. It is possible to convert a reversible inhibitor to an irreversible inhibitor by incorporating an irreversible “warhead” into what would otherwise be a reversible inhibitor.

The reversibility of ICE/CED-3 inhibition is generally a function of the electronegative group in the molecule. When the electronegative group is a diazoalkyl ketone, the inhibition of ICE activity is irreversible and the compound is an irreversible inhibitor. When the electronegative group is an aldehyde, the inhibition of CASPASE-1 is reversible and the inhibitor is a reversible inhibitor.

A compound of the invention preferably has an aldehyde, a diazoalkyl ketone, a haloalkyl ketone, or acyloxymethyl ketone. As used herein in reference to an electronegative group, “alkyl” refers to linear or branched chain radicals having 1-3 carbon atoms, which may be optionally substituted. Representative alkyl groups include methyl, ethyl, propyl and the like. Optionally, the electronegative group is an aldehyde, fluoromethyl (CH₂F) ketone, or acyloxymethyl ketone.

The compounds of the present invention are made by techniques generally corresponding to methods known and readily apparent to those of skill in the art. See, e.g., Kettner et al., Arch. Biochem. Biophys. 162:56 (1974); U.S. Pat. Nos. 4,582,821; 4,644,055; Kettner et al. Arch. Biochem. Biophys. 165:739, (1974); Dakin and West, J. Biol. Chem., 78:91 (1928); Rasnick, D., Anal. Biochem. 149:461 (1985); Revesz, L., Tetrahedron Lett., 35:9693 (1994). Exemplary indolyl dipeptide and tricyclic compounds are provided herein.

Compounds having a non-fluoro, haloalkyl ketone electronegative leaving group are preferably synthesized in accordance with the Kettner procedure. An N-blocked amino acid or peptide is reacted with N-methylmorpholine and an alkyl, non-fluoro haloformate to generate a peptide-acid anhydride. The anhydride is then reacted with a diazoalkane in an inert, aprotonic solvent to form a peptide-diazomethane ketone. The diazomethane ketone is then reacted with an anhydrous solution of HCl, HBr or HI to produce the desired N-blocked, C-terminal haloalkyl ketone peptide or amino acid.

Compounds having a fluoromethyl electronegative leaving group are preferably synthesized by the Revesz procedure. An N-blocked peptide or amino acid is reacted with t-butyl (3-amino-4-hydroxy-5-fluoro) pentanoate in the presence of a standard peptide coupling agent such as dicyclohexylcarbodiimide-hydroxy-benztriazole. The resulting product is oxidized to the corresponding ketone by either Severn or Dess-Martin oxidation. Finally, deprotection of the t-butylester with trifluoracetic acid gives the corresponding carboxylic acid.

Compounds having a fluoroalkyl ketone electronegative leaving group can be extended in the N-terminus direction by removing the N-terminal blocking group and coupling the deprotected compound with other protected amino acids. Bodanszky, The Practice of Peptide Synthesis, Springer-Verlag, Berlin, 1984. Alternatively, deprotected compounds are acetylated to yield compounds having an N-terminal acetyl protecting group. Stewart et al., Solid Phase Peptide Synthesis, Pierce Chemical Co., Rockford, Ill., 1984.

Compositions and Kits

The present invention also provides kits adapted for the preservation of antigenicity of an infected tissue sample. Such kits include an appropriate apoptotic inhibitory reagent as well as instructions for use. In certain embodiments, the kits also comprise an appropriate container for either retaining the anti-apoptotic reagent and/or for collection of the sample to be contacted.

Compositions of this invention comprise any of the compounds of the present invention, and salts thereof, with any acceptable carrier, adjuvant or vehicle such as pharmaceutically acceptable carriers. Acceptable carriers, adjuvants and vehicles that may be used in the compositions of this invention include, but are not limited to, alumina, aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene-polyoxypropylene-block polymers, polyethylene glycol and wool fat, and the like.

The compositions of the present invention may include any of the compounds mentioned above or any apoptosis inhibitor in combination with other protease inhibitors to form a cocktail to inhibit protein degradation thereby. Such compositions are useful in preserving or enhancing antigenicity of infectious agent markers in a tissue sample, such as virally infected cells.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The following examples are intended to illustrate, but not limit the invention. While they are typical of those that might be used, other procedures known to those skilled in the art may alternatively be used.

In the following Examples, proton NMR spectra were obtained at 300 MHZ; chemical shifts are quoted downfield from internal tetramethylsilane.

### EXAMPLE 1

### Assays for Inhibition of ICE/CED-3 Protease Family Activity

A. Determination of IC₅₀ Values

Fluorescence enzyme assays detecting the activity of the compounds of Formula I utilizing the recombinant ICE and caspase-3 enzymes were performed essentially according to Thornberry et al. (Nature 356:768-774 (1992)) and Nicholson et al. Nature 376:37-43 (1995)) respectively, (herein incorporated by reference) in 96 well microtiter plates. The substrate for these assays was Acetyl-Tyr-Val-Ala-Asp-amino-4-methylcoumarin (AMC) for the ICE assay and Acetyl-Asp-Glu-Val-Asp-amino-4-methylcoumarin for the caspase-3 and Caspase-8 assay. Enzyme reactions were run in ICE buffer (25 mM HEPES, 1 mM EDTA, 0.1% CHAPS, 10% sucrose, pH 7.5) containing 2 mM DTT at room temperature in duplicate. The assays were performed by mixing the following components:

50 μl of either ICE, Caspase-6, Caspase-8 or caspase-3 (18.8, 38, 8.1 and 0.153 nM concentrations, respectively) or Caspase-7 (1 unit) enzyme in ICE buffer containing either 8.0 (ICE, Caspase-6, Caspase-7, caspase-3) or 20 (Caspase-8) mM DTT;

50 μl of either the compound of Formula I or ICE buffer (control); and

100 μl of 20 μM substrate.

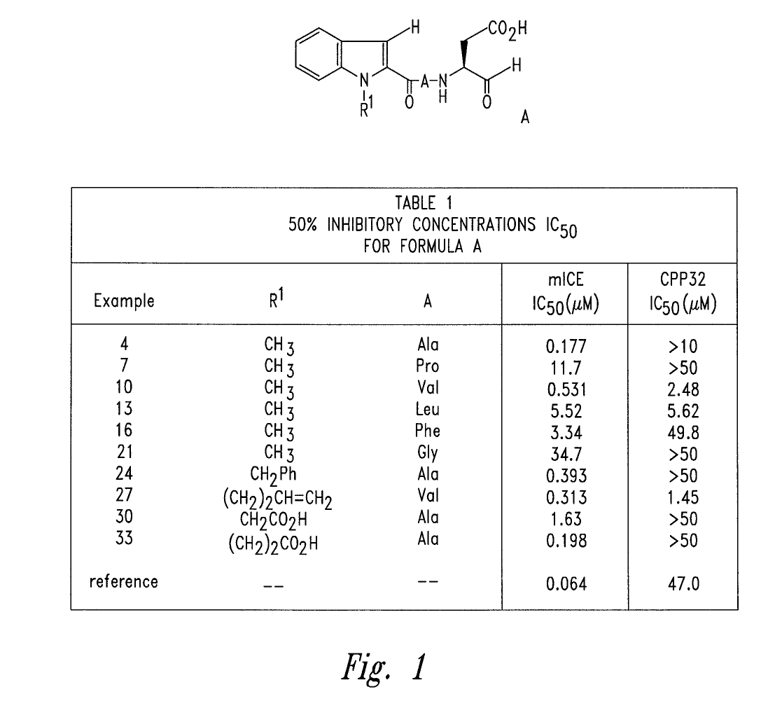

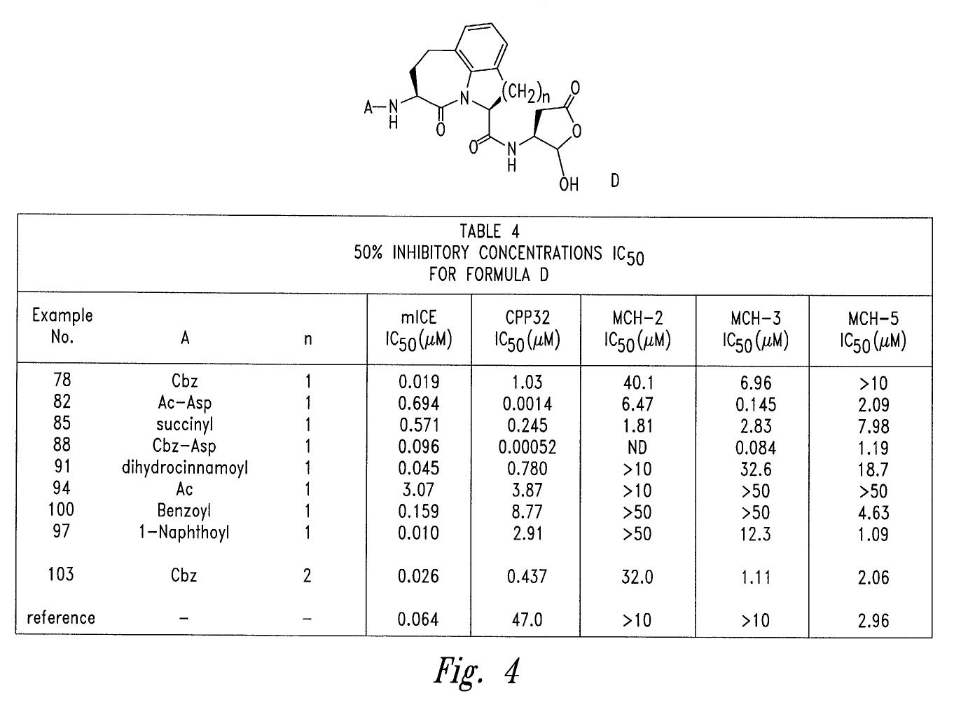

The enzyme and the compound of Formula I to be assayed were preincubated in the microtitre plate wells for 30 minutes at room temperature prior to the addition of substrate to initiate the reaction. Fluorescent AMC product formation was monitored for one hour at room temperature by measuring the fluorescence emission at 460 nm using an excitation wavelength of 360 nm. The fluorescence change in duplicate (control) wells were averaged and the mean values were plotted as a function of inhibitor concentration to determine the inhibitor concentration producing 50% inhibition (IC₅₀). The results are set forth in FIGS. 1 and 4.

The reference compound for this assay was Cbz-ValAlaAsp-H and the values are denoted in FIGS. 1 and 4 as “Reference.”

B. Determination of the Dissociation Constant K, and Irreversible Rate Constant for k₃ Irreversible Inhibitors

For the irreversible inhibition of a ICE/CED-3 Family Protease enzyme with a competitive irreversible inhibitor; using the model represented by the following formulas:

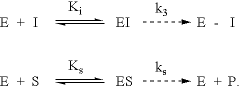

The product formation at time t may be expressed as:

$$[ P ] t = {[ E ]}^{T} ( \frac{[ S ] {K}_{1}}{[ I ] {K}_{S}} ) ( \frac{{k}_{S}}{{k}_{3}} ) \\ [ 1 - e - {k}_{3} t / ( 1 + \frac{{K}_{1}}{[ I ]} ( 1 + \frac{[ S ]}{{K}_{S}} ) ) ] \qquad ( Equation 1 )$$

where E, I, EI, and E-I denote the active enzyme, inhibitor, non-covalent enzyme-inhibitor complex and covalent enzyme-inhibitor adduct, respectively. The K₁ value is the overall dissociation constant of reversible binding steps, and k₃ is the irreversible rate constant. The [S] and Kₛ values are the substrate concentration and the dissociation constant of the substrate bound to the enzyme, respectively.

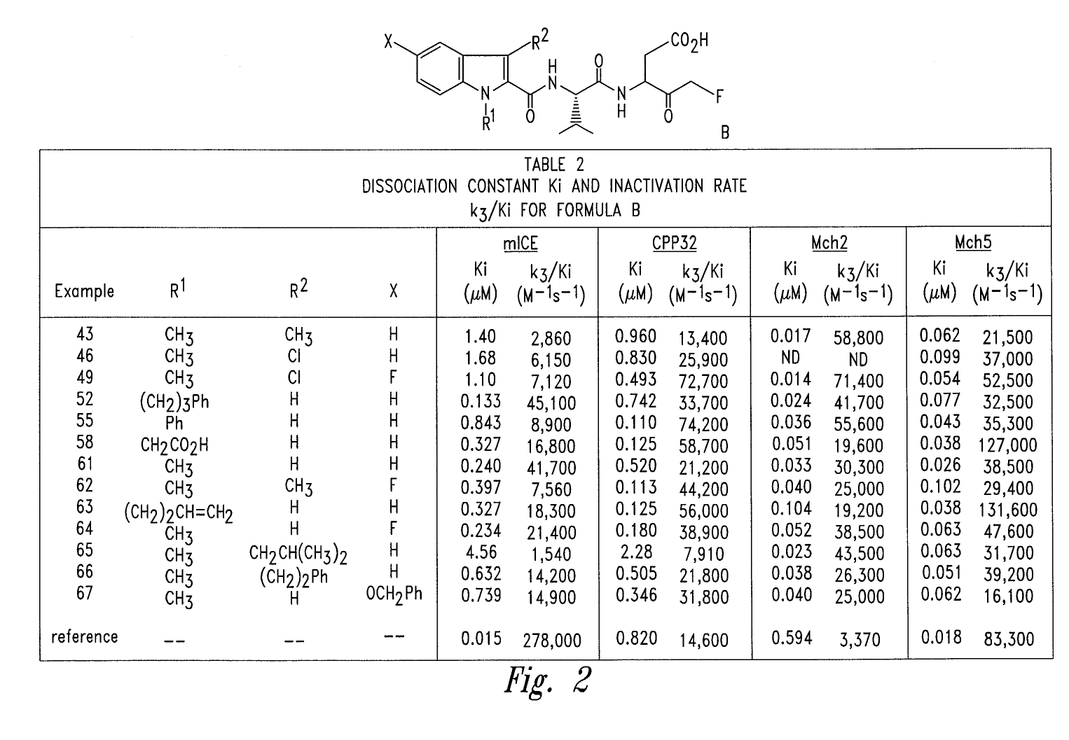

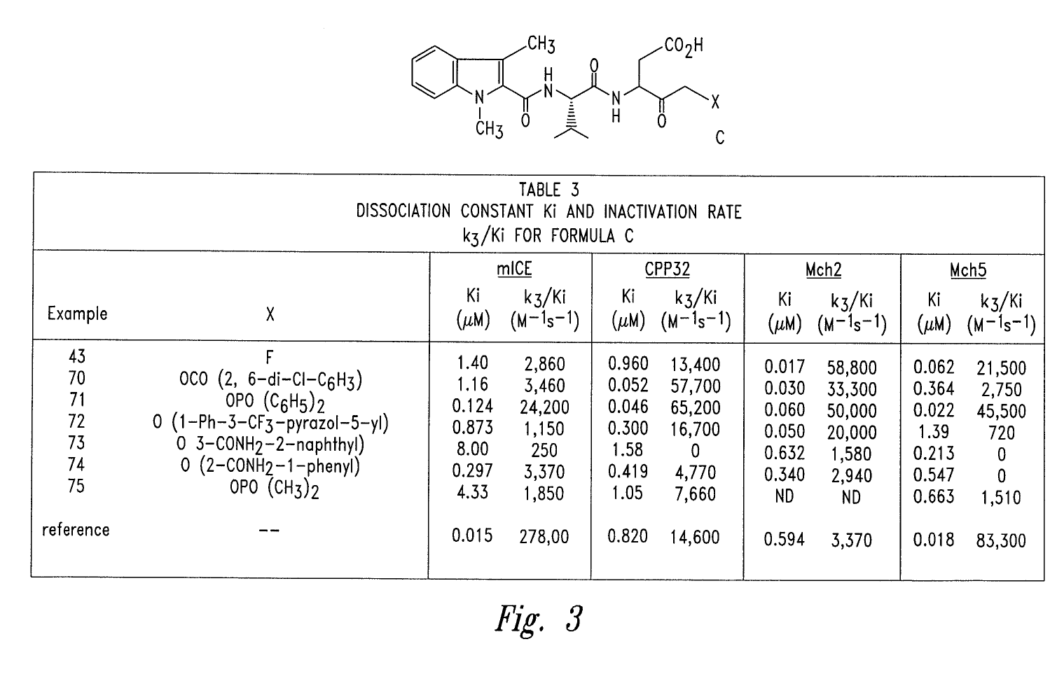

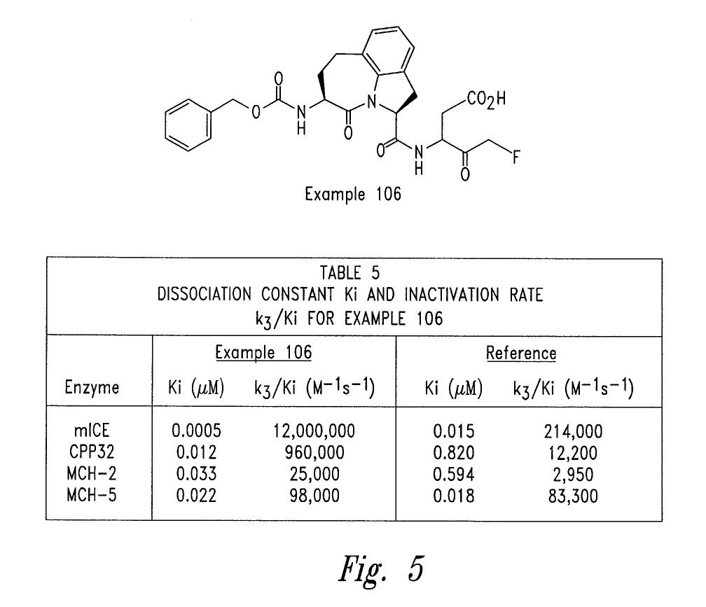

The above equations were used to determine the K₁ and k₃ values of a given inhibitor bound to a ICE/CED-3 family protease. Thus, a continuous assay was run for sixty minutes at various concentrations of the inhibitor and the substrate. The assay was formulated essentially the same as described above for generating the data in Table 1, except that the reaction was initiated by adding the enzyme to the substrate-inhibitor mixture. The Kᵢ and k₃ values were obtained by simulating the product AMC formation as a function of time according to Equation I. The results of this second assay are set forth below in Tables 2, 3 and 5 (FIGS. 2, 3 and 5).

The reference compound for this assay was Cbz-ValAlaAsp-CH₂F and the values are denoted in Tables 2, 3 and 5 as “Reference.”

### EXAMPLE 2

### 3 S)-3-((1-methylindole-2-carbonyl)alaninyl]amino-4-oxobutanoic Acid, T-Butyl Ester Semicarbazone

1-Methylindole-2-carboxylic acid (107 mg, 0.6 mmol) and (3S)-3-(alaninyl)-amino-4-oxobutanoic acid, t-butyl ester semicarbazone (188 mg, 96%, 0.6 mmol) were dissolved in DMF (2 mL) then both 1-hydroxybenzotriazole-hydrate (96 mg, 0.63 mmol) and 1-(3-dimethylaminopropyl)-3-ethyl carbodiimide hydrochloride (EDAC) (161 mg, 0.84 mmol) was added to the resultant mixture under a nitrogen atmosphere at 0° C. Stirring was continued for 1 hour at 0° C. and an additional 20 hours at room temperature. The reaction mixture was diluted with ethyl acetate, washed successively with saturated aqueous sodium bicarbonate solution and brine, dried over sodium sulfate and concentrated to give a yellow solid. Trituration of the solid with ether afforded the title product as a slightly yellow powder (213 mg, 77%). TLC: (methanol/methylene chloride: 1/9, silica gel): R_{f}=0.47; ¹H NMR (CDCl₃+CD₃OD): δ 7.96 (d, J=8.0, 1H), 7.57-7.67 (m, 2H), 7.31-7.42 (m, 2H), 7.13-7.19 (m, 2H), 7.06 (s, 1H), 4.91 (m, 1H), 4.65 (q, J=7.1, 1H), 4.01 (s, 3H), 2.59-2.78 (m, J=5.6, 15.7, 2H), 1.49 (d, J=7.1, 3H), 1.39 (s, 9H).

### EXAMPLE 3

### (3S)-3-[(1-methylindole-2-carbonyl)alaninyl]amino-4-oxobutanoic Acid, Semicarbazone

(3S)-3-[(1-Methylindole-2-carbonyl)alaninyl]amino-4-oxobutanoic acid, t-butyl ester semicarbazone (127 mg, 0.28 mmol) was suspended in anisole (0.2 mL) and methylene chloride (2 mL) and the suspension was treated with trifluroacetic acid (TFA) (1 mL). The resulting solution was stirred for 2 hours under a nitrogen atmosphere at room temperature. The reaction mixture was then concentrated and chased with methylene chloride to give a purple foam. Trituration of the foam with ether gave the title product as a purple powder (108 mg, 97%). TLC: (methylene chloride:methanol:acetic acid, 20:1:1, silica gel): R_{f}=0.27; ¹H NMR (CD₃OD): δ 7.62 (d, J=8.0, 1H), 7.44 (d, J=8.2, 1H), 7.24-7.32 (m, 2H), 7.07-7.13 (m, 2H), 4.91 (m, 1H), 4.56 (q, J=7.1, 1H), 3.98 (s, 3H), 2.78 (d, J=6.5, 2H), 1.49 (d, J=7.3, 3H).

### EXAMPLE 4

### (3S)-3-[(1-methylindole-2-carbonyl)alaninyl]amino-4-oxobutanoic Acid

(3S)-3-[(1-Methylindole-2-carbonyl)alaninyl]amino-4-oxobutanoic acid, semicarbazone (87 mg, 0.22 mmol) was dissolved in methanol (3 mL), formaldehyde (1 mL, 37% wt. aq) and acetic acid (1 mL) and the resultant mixture was stirred for 4 hours under a nitrogen atmosphere at room temperature. The reaction mixture was diluted with water and extracted twice with ethyl acetate. The ethyl acetate solution was washed with brine, dried over sodium sulfate and concentrated to give a glassy material which was triturated with ether to afford the title product as a gray powder (24 mg, 32%). TLC: (methylene chloride:methanol:acetic acid, 20:1:1, silica gel): R_{f}=0.44; ¹H NMR (CD₃OD): δ 7.62 (d, J=8.0, 1H), 7.44 (dd, J=0.8, 8.4, 1H), 7.26-7.32 (m, 1H), 7.08-7.13 (m, 2H), 4.63-4.53 (m, 2H), 4.31 (m, 1H), 3.99 (s, 3H), 2.48-2.73 (m, 2H), 1.46 (7.1, 3H).

### EXAMPLE 5

### (3S)-3-[(1-methylindole-2-carbonyl]prolinyl]amino-4-oxobutanoic Acid, T-Butyl Ester Semicarbazone

1-Methylindole-2-carboxylic acid (102 mg, 0.58 mmol) and (3S)-3-(prolinyl)amino-4-oxobutanoic acid, t-butyl ester semicarbazone (189 mg, 0.58 mmol) were dissolved in methylene chloride (2 mL) and DMF (1 mL) and then both 4-dimethylamino pyridine (DMAP) (71 mg, 0.58 mmol) and EDAC (155 mg, 0.81 mmol) were added to the mixture under a nitrogen atmosphere at 0° C. Stirring was continued for 1 hour at 0° C. and an additional 2 hours at room temperature. The reaction mixture was partitioned between ethyl acetate and 5% KHSO₄ solution. The ethyl acetate solution was washed with saturated sodium bicarbonate solution and brine, dried over sodium sulfate and concentrated to give 153 mg of brown foam. The foam was purified by flash chromatograph on silica gel using 2% methanol-methylene chloride as the eluant to give the title product as a light brown foam (50 mg). TLC: (methanol/methylene chloride: 5/95, silica gel): R_{f} 0.27; ¹H NMR (CDCl₃+CD₃OD): δ 8.87 (bs, 1H), 7.63 (d, J=7.7, 1H), 7.38-7.50 (m, 2H), 7.17-7.13 (m, 1H), 6.85 (bs, 1H), 4.90-4.81 (m, 2H), 3.92-3.74 (m, 5H), 2.78-1.93 (m, 6H), 1.37 (s, 9H).

### EXAMPLE 6

### (3S)-3-[(1-methylindole-2-carbonyl)prolinyl]amino-4-oxobutanoic Acid Semicarbazone

(3S)-3-[(1-Methylindole-2-carbonyl)prolinyl]amino-4-oxobutanoic acid, t-butyl ester semicarbazone (50 mg, 0.1 mmol) was dissolved in anisole (0.2 mL) and methylene chloride (2 mL) and the resultant solution was treated with TFA (1 mL). This reaction mixture was then stirred for 1 hour under a nitrogen atmosphere at room temperature. The reaction mixture was concentrated in vacuo and chased with methylene chloride to give a purple film. The film was triturated with ether to afford the title product as a purple powder (47 mg). TLC: (methylene chloride:methanol:acetic acid, 20:1:1, silica gel): R_{f}=0.18; ¹H NMR (CD₃OD): δ 7.63-6.93 (m, 6H), 6.67 (bs, 1H), 4.89-4.50 (m, 2H), 3.86-3.74 (m, 5H), 2.82-2.74 (m, 2H), 2.40-2.30 (m, 1H), 2.15-1.90 (m, 3H).

### EXAMPLE 7

### (3S)-3-[(1-methylindole-2-carbonyl)prolinyl]amino-4-oxobutanoic Acid

(3S)-3-[(1-Methylindole-2-carbonyl)prolinyl]amino-4-oxobutanoic acid, semicarbazone (87 mg, 0.22) mmol) was dissolved in methanol (3 mL), formaldehyde (1 mL, 37% wt. aq) and acetic acid (1 mL) and the resulting mixture was stirred for 4 hours under a nitrogen atmosphere at room temperature. The reaction mixture was concentrated in vacuo, diluted with water, and extracted twice with ethyl acetate. The ethyl acetate solution was washed with brine, dried over sodium sulfate and concentrated to give brown oil (22 mg) which was triturated with ether to afford the title product as a light brown powder (8 mg). TLC: (methylene chloride:methanol:acetic acid, 20:1:1, silica gel): R_{f}=0.28; MS (EI) for C₁₉H₂₁N₃O₅+H⁺=372; C₁₉H₂₁N₃O₅−H⁺=370).

### EXAMPLE 8

### (3S)-3-[(1-methylindole-2-carbonyl)valinyl]amino-4-oxobutanoic Acid, T-Butyl Ester Semicarbazone

1-Methylindole-2-carboxylic acid (88 mg, 0.5 mmol) and (3S)-3-(Valinyl)amino-4-oxobutanoic acid, t-butyl ester semicarbazone (163 mg, 0.5 mmol) were dissolved in DMF (1 mL) and methylene chloride (2 mL) then both DMAP (61 mg, 0.50 mmol) and EDAC (134 mg, 0.7 mmol) were added to the solution under a nitrogen atmosphere at 0° C. Stirring was continued for 1 hour at 0° C. and an additional 4 hours at room temperature. The reaction mixture was partitioned between ethyl acetate and 5% KHSO₄ solution. The ethyl acetate solution was washed successively with 5% KHSO₄ solution, saturated sodium bicarbonate solution and brine solutions, dried over sodium sulfate, and concentrated to give a yellow foam. Trituration of the foam with ether afforded the title product as a slightly yellow powder (203 mg, 86%). TLC: (methanol/methylene chloride:5/95, silica gel): R_{f}=0.17.

### EXAMPLE 9

### (3S)-3-[(1-methylindole-2-carbonyl)valinyl]amino-4-oxobutanoic Acid Semicarbazone

(3S)-3-[(1-Methylindole-2-carbonyl)valinyl]amino-4-oxobutanoic acid, t-butyl ester semicarbazone (170 mg, 0.36 mmol) was dissolved in anisole (0.2 mL) and methylene chloride (2 mL) and the resulting solution was treated with TFA (1 mL). The resulting solution was stirred for 3.5 hours under a nitrogen atmosphere at room temperature. The reaction mixture was concentrated in vacuo and chased with methylene chloride to give a purple foam. Trituration of the foam with ether afforded the title product as a solid purple powder (133 mg, 89%).

### EXAMPLE 10

### (3S)-3-[1-methylindole-2-carbonyl)valinyl]amino-4-oxobutanoic Acid

(3S)-3-[(1-Methylindole-2-carbonyl)valinyl]amino-4-oxobutanoic acid, semicarbazone (136 mg, 0.33 mmol) was dissolved in methanol (3 mL), formaldehyde (1 mL, 37% wt. aq) and acetic acid (1 mL) and the resulting mixture was stirred for 5 hours under a nitrogen atmosphere at room temperature. The reaction mixture was concentrated in vacuo diluted with water, and extracted twice with ethyl acetate. The combined ethyl acetate solutions were washed with brine, dried over sodium sulfate and concentrated in vacuo to give a purple foam which was triturated with ether to afford the title product as a purple powder (40 mg, 33%). TLC: (methylene chloride:methanol:acetic acid, 20:1:1, silica gel): R_{f}=0.36; MS (EI) for C₁₉H₂₃N₃O₅+H⁺=374; C₁₉H₂₃N₃O₅−H⁺=372).

### EXAMPLE 11

### (3S)-3-[(1-methylindole-2-carbonyl)leucinyl]amino-4-oxobutanoic Acid, T-Butyl Ester Semicarbazone

1-Methylindole-2-carboxylic acid (70 mg, 0.4 mmol) and 3(S)-(Leucinyl)amino-4-oxobutanoic acid, t-butyl ester semicarbazone (131 mg, 0.4 mmol) were dissolved in methylene chloride (2 mL) and both DMAP (49 mg, 0.40 mmol) and EDAC (107 mg, 0.56 mmol) were added to the solution under a nitrogen atmosphere at 0° C. Stirring was continued for 1 hour at 0° C. and an additional 3 hours at room temperature. The reaction mixture was partitioned between ethyl acetate and 5% KHSO₄ solution. The ethyl acetate solution was washed successively with 5% KHSO₄ solution, saturated with sodium bicarbonate solution (2×) and brine, dried over sodium sulfate, and concentrated in vacuo to give a crude solid. Trituration of me solid with ether afforded the title product as a white powder (156 mg, 80%). TLC: (methanol/methylene chloride: 5/95, silica gel): R_{f}=0.42; ¹H NMR (CDCl₃+CD₃OD): δ 8.18 (s, 1H), 7.66-7.11 (m, 6H), 6.97 (s, 1H), 6.32 (d, J=7.7, 1H), 4.95-4.88 (m, 1H), 4.70-4.62 (m, 1H), 4.03 (s, 3H), 2.82-2.56 (m, 2H), 1.87-1.58 (m, 3H), 1.38 (9H), 1.00 (t, J-6.3, 6H).

### EXAMPLE 12

### (3S)-3-[(1-methylindole-2-carbonyl)leucinyl]amino-4-oxobutanoic Acid, Semicarbazone

(3S)-3-[(1-Methylindole-2-carbonyl)leucinyl]amino-4-oxobutanoic acid, t-butyl ester semicarbazone (132 mg, 0.27 mmol) was dissolved in anisole (0.2 mL) and methylene chloride (2 mL) and the resulting solution was treated with TFA (1 mL). The resulting solution was stirred for 3 hours under a nitrogen atmosphere at room temperature. The reaction mixture was concentrated in vacuo and chased with methylene chloride to give a pink foam. Trituration of the foam with ether afforded the title product as a pink powder (108 mg, 92%). TLC: (methylene chloride:methanol:acetic acid, 20:1:1, silica gel): R_{f}=0.22; ¹H NMR (CD₃OD): δ 7.62 (dt, J=8.0, 1.1, 1H), 7.45 (dd, J=8.5, 0.8, 1H), 7.32-7.23 (m, 2H), 7.13-7.08 (m, 2H), 4.94-4.89 (m, 1H), 4.64-4.59 (m, 1H), 3.98 (s, 3H), 2.78 (d, J=6.2, 2H), 1.82-1.70 (m, 3H), 1.02 (d, J=6.0, 3H), 0.99 (d, J=6.3, 3H).

### EXAMPLE 13

### (3S)-3-[(1-methylindole-2-carbonyl)leucinyl]amino-4-oxobutanoic Acid

(3S)-3-[(1-Methylindole-2-carbonyl)leucinyl]amino-4-oxobutanoic acid, semicarbazone (90 mg, 0.21 mmol) was dissolved in methanol (3 mL), formaldehyde (1 mL, 37% wt. aq) and acetic acid (1 mL) and the resulting solution was stirred for 7 hours under a nitrogen atmosphere at room temperature. The reaction mixture was concentrated in vacuo, diluted with water, and extracted twice with ethyl acetate. The ethyl acetate solution was washed with brine, dried over sodium sulfate, and concentrated in vacuo to give a purple foam which was triturated with ether to afford the title product as a purple powder (35 mg, 43%). TLC: (methylene chloride:methanol:acetic acid, 20:1:1, silica gel): R_{f}=0.45; MS(EI) for C₂₀H₂₅N₃O₅; M+H⁺=388; M−H⁺=386.

### EXAMPLE 14

### (3S)-3-[(1-methylindole-2-carbonyl)phenylalaninyl]amino-4-oxobutanoic Acid, T-Butyl Ester Semicarbazone

1-Methylindole-2-carboxylic acid (72 mg, 0.41 mmol) and 3(S)-(phenylalaninyl]amino-4-oxobutanoic acid, t-butyl ester semicarbazone (154 mg, 0.41 mmol) were dissolved in methylene chloride (2 mL) and both DMAP (53 mg, 0.43 mmol) and EDAC (109 mg, 0.57 mmol) were added to the solution under a nitrogen atmosphere at 0° C. Stirring was continued for 1 hour at 0° C. and an additional 4 hours at room temperature. The reaction mixture was partitioned between ethyl acetate and 5% KHSO₄ solution, successively, dried over sodium sulfate, and concentrate to give a white solid. Trituration of the solid with ether afforded the title product as a white powder (179 mg, 82%). TLC: (methanol/methylene chloride: 5/95, silica gel): R_{f}=0.44.

### EXAMPLE 15

### (3S)-3-[(1-methylindole-2-carbonyl)phenylalaninyl]amino-4-oxobutanoic Acid, Semicarbazone

(3S)-3-[(1-Methylindole-2-carbonyl) phenylalaninyl]amino-4-oxobutanoic acid, t-butyl ester semicarbazone (154 mg, 0.30 mmol) was dissolved in anisole (0.2 mL) and methylene chloride (2 mL) and the resulting solution was treated with TFA (1 mL). The resulting solution was stirred for 4 hours under a nitrogen atmosphere at room temperature. The reaction mixture was concentrated in vacuo and azeotroped with methylene chloride to give a purple solid. Trituration of the solid with ether afforded the title product as a purple powder (141 mg, 100%). TLC: (methylene chloride:methanol:acetic acid, 20:1:1, silica gel): R_{f}=0.25.

### EXAMPLE 16

### (3S)-3-[(1-methylindole-2-carbonyl)phenylalaninyl]amino-4-oxobutanoic Acid

(3S)-3-[(1-Methylindole-2-carbonyl)phenyl-alaninyl]amino-4-oxobutanoic acid, semicarbazone (116 mg, 0.25 mmol) were dissolved in methanol (3 mL), formaldehyde (1 mL, 37% wt. aq) and acetic acid (1 mL) and the resulting solution was stirred for 9 hours under a nitrogen atmosphere at room temperature. The reaction mixture was concentrated in vacuo, diluted with water, and extracted twice with ethyl acetate. The ethyl acetate solution was washed with brine, dried over sodium sulfate and concentrated to give a crude product which was triturated with ether to afford the title product as a brown powder (26 mg, 25%). TLC: (methylene chloride:methanol:acetic acid, 20:1:1, silica gel): R_{f}=0.33; MS(EI) for C₂₃H₂₁N₃O₅; M+H⁺=422; M−H⁺=420.

### EXAMPLE 17

### (1-methylindole-2-carbonyl)glycine, Methyl Ester

DMAP (1.222 g, 0.01 mol) and EDAC (2.680 g, 0.014 mol) were added as solids to a solution of 1-methylindole-2-carboxylic acid (1.752 g, 0.01 mol) and glycine methyl ester hydrochloride (1.256 g, 0.01 mol) in methylene chloride (30 mL) and DMF (5 mL) under a nitrogen atmosphere at 0° C. Stirring was continued for 1 hour at 0° C. and then for 3 hours at room temperature. The reaction mixture was partitioned with ethyl acetate and 5% KHSO₄ solution and the aqueous layer was extracted with ethyl acetate. The combined ethyl acetate solution was washed with 5% KHSO₄ solution, saturated sodium bicarbonate solution (2×) solution and brine, dried over sodium sulfate, and concentrated to give a purple powder as crude product. Trituration of the powder with ether afforded the title product (1.734 mg, 70%). TLC: (methanol/methylene chloride 1:9): R_{f}=0.61; ¹H NMR (CDCl₃): δ 7.65 (dt, J=8.0, 1.1, 1H), 7.41-7.31 (m, 2H), 7.16 (dd, J=6.6, 1.4, 1H) 6.96 (d, J=0.5, 1H), 6.67 (bs, 1H), 4.25 (d, J=5.2, 2H), 4.05 (s, 3H), 3.82 (s, 3H).

### EXAMPLE 18

### (1-methylindole-2-carbonyl)Glycine

(1-Methylindole-2-carbonyl)glycine methyl ester (1.687 g, 6.85 mmol) was dissolved in 1,4-dioxane (10 mL) and was treated with 1 N lithium hydroxide (7.0 mL, aq) with stirring. The reaction mixture turned clear immediately and was acidified with 1N HCl and concentrated to remove 1,4-dioxane to result in a purple precipitate. The precipitate was filtered, washed with water, and dried in vacuo to give the title product as a purple powder (1.482 g, 93%). TLC: (methylene chloride:methanol:acetic acid, 20:1:1, silica gel): R_{f}=0.28; ¹H NMR (CD₃OD): δ 7.61 (dt, J=8.2, 1H), 7.44 (dd, J=8.5, 0.8, 1H), 7.32-7.26 (m, 1H), 7.13-7.09 (m, 1H), 7.04 (s, 1H), 4.08 (s, 2H), 3.99 (s, 3H).

### EXAMPLE 19

### (3S)-3-[(1-methylindole-2-carbonyl)glycine]amino-4-oxobutanoic Acid, T-Butyl Ester Semicarbazone

(1-Methylindole-2-carbonyl)glycine (186 mg, 0.8 mmol) was dissolved in methylene chloride (5 mL) and DMF (1 mL) and the resulting solution was treated with 1-hydroxybenzotriazole hydrate (129 mg, 0.84 mmol) and EDAC (215 mg, 1.12 mmol) under a nitrogen atmosphere and the reaction mixture stirred for 10 minutes at 0° C. 3(S)-Amino-4-oxobutanoic acid, t-butyl ester semicarbazone p-toluenesulfate (312 mg, 0.8 mmol) followed by N-methylmorpholine (0.09 mL, 0.8 mmol), were added to the reaction mixture and the mixture was stirred for 1 hour at 0° C. and an additional 4 hours at room temperature. The reaction mixture was partitioned between ethyl acetate and 5% KHSO₄, and the product precipitated out during the work-up. The white precipitate from the aqueous portion was obtained by filtration and washing with water and ether. Another portion of white precipitate was obtained by concentration of the organic layer and trituration of the residue with ether. The combined precipitate was the title product (297 mg, 66%). TLC: (methanol/methylene chloride: 1/9, silica gel): R_{f}=0.42; ¹H NMR, (CDCl₃) δ 7.65 (d, J=8.0, 1H), 7.41-7.34 (m, 2H), 7.19-7.13 (m, 2H), 7.05 (d, J=0.5, 1H), 4.95-4.93 (m, 1H), 4.08 (s, 2H), 4.03 (s, 3H), 2.79-2.59 (m, 2H), 1.41 (s, 9H).

### EXAMPLE 20

### (3S)-3-[(1-methylindole-2-carbonyl)glycinyl]amino-4-oxobutanoic Acid, Semicarbazone

(3S)-3-[(1-Methylindole-2-carbonyl)glycinyl]amino-4-oxobutanoic acid, t-butyl ester semicarbazone (118 mg, 0.26 mmol) was dissolved in anisole (0.2 mL) and methylene chloride (2 mL) and the resulting solution was treated with TFA (1 mL). The resulting solution was stirred for 3 hours under a nitrogen atmosphere at room temperature. The reaction mixture was concentrated in vacuo and chased with methylene chloride to give a green solid. Trituration of the solid with ether afforded the title product as a green powder (88 mg, 87%). TLC: (methylene chloride:methanol:acetic acid, 20:1:1, silica gel): R_{f}=0.47; ¹H NMR (CD₃OD): δ 7.63-7.08 (m, 6H), 4.95 (m, 1H), 4.05 (s, 2H), 4.01 (s, 3H), 3.77 (d, J=5.8, 2H).

### EXAMPLE 21

### (3S)-3-[(1-methylindole-2-carbonyl]glycinyl]-amino-4-oxobutanoic Acid

(3S)-3-[(1-Methylindole-2-carbonyl)glycinyl]amino-4-oxobutanoic acid, semicarbazone (76 mg, 0.20 mmol) was dissolved in a mixture of methanol (3 mL), formaldehyde (1 mL, 37% wt. aq) and acetic acid (1 mL) and the mixture was stirred for 6 hours under a nitrogen atmosphere at room temperature. The reaction mixture was concentrated in vacuo, diluted with water, extracted twice with ethyl acetate. The combined ethyl acetate solutions were washed with brine, dried over sodium sulfate, and concentrated to give a crude product which was triturated with ether to afford the title product as a light yellow powder (29 mg, 44%). TLC: (methylene chloride:methanol:acetic acid, 8:1:1, silica gel): R_{f}=0.61; MS(EI) for C₁₆H₁₇N₃O₅:M+H⁺,330. ¹H NMR (CD₃OD): δ 7.73-7.08 (m, 5H), 4.90-3.8 (m, 7H), 2.72-2.47 (m, 2H).

### EXAMPLE 22

### (3S)-3-[(1-benzylindole-2-carbonyl)alaninyl]amino-4-oxobutanoic Acid, T-Butyl Ester Semicarbazone

1-Benzylindole-2-carboxylic acid (477 mg, 1.9 mmol) and 3(S)-(alaninyl)amino-4-oxobutanoic acid, t-butyl ester semicarbazone (581 mg, 1.9 mmol) were dissolved in methylene chloride (8 mL) and both DMAP (232 mg, 1.9 mmol) and EDAC (498 mg, 2.6 mmol) were added to the solution under a nitrogen atmosphere at 0° C. The resultant solution was stirred for 1 hour at 0° C. and an additional 2 hours at room temperature. The reaction mixture was diluted with ethyl acetate, washed successively with saturated sodium bicarbonate solution and brine, dried over sodium sulfate, and concentrated to give a yellow foam. Flash column chromatographic purification of the foam (silica gel, methanol/methylene chloride 2-5%) afforded the title product as a white powder (570 mg, 56%). TLC: (methanol/methylene chloride:1/9, silica gel): R_{f}=0.38; ¹H NMR (CDCl₃): δ 8.60 (bs, 1H), 7.67 (dd, J=8.0, 1.1, 1H), 7.50 (d, J=8.0, 1H), 7.33-7.01 (m, 8H), 6.79 (d, J=7.4, 1H), 5.78 (s, 2H), 4.87-4.83 (m, 1H), 4.67-4.62 (m, 1H), 2.73-2.43 (m, 2H), 1.46 (d, J=7.1, 3H), 1.39 (s, 9H).

### EXAMPLE 23

### (3S)-3-[(1-benzylindole-2-carbonyl)alaninyl]amino-4-oxobutanoic Acid, Semicarbazone

(3S)-3-[(1-Benzylindole-2-carbonyl)alaninyl]amino-4-oxobutanoic acid, t-butyl ester semicarbazone (247 mg, 0.46 mmol) was dissolved in anisole (0.5 mL) and methylene chloride (2 mL) and the resultant mixture was treated with TFA (1 mL). The resulting solution was stirred for 3.5 hours under a nitrogen atmosphere at room temperature. The reaction mixture was concentrated and chased with methylene chloride to give a light green solid. Trituration of the solid with ether afforded the title product as a green powder (215 mg, 98%). TLC: (methylene chloride:methanol:acetic acid, 8:1:1, silica gel): R_{f}=0.50; ¹H NMR (CD₃OD): δ 8.26 (d, J=8.0, 1H), 7.65 (d, J=8.0, 1H), 7.39 (dd, J=8.5, 0.8, 1H), 7.26-7.01 (m, 8H), 5.79 (d, J=7.4, 2H), 4.56-4.49 (m, 1H), 2.77-2.62 (m, 2H), 1.43 (d, J=7.4, 3H).

### EXAMPLE 24

### (3S)-3-[(1-benzylindole-2-carbonyl)alaninyl]amino-4-oxobutanoic Acid

(3S)-3-[(1-Benzylindole-2-carbonyl)alaninyl]amino-4-oxobutanoic acid, semicarbazone (176 mg, 0.37 mmol) was dissolved in methanol (4.5 mL), formaldehyde (1.5 mL, 37% wt. aq) and acetic acid (1.5 mL) and the resulting mixture was stirred for 4 hours under a nitrogen atmosphere at room temperature. The reaction mixture was concentrated in vacuo, diluted with water, and extracted twice with ethyl acetate. The ethyl acetate solution was washed with brine, dried over sodium sulfate, and concentrated to give a crude product which was triturated with ether to afford the title product as a light green powder (113 mg, 72%). TLC: (methylene chloride:methanol:acetic acid, 20:1:1, silica gel): R_{f}=0.38; MS for C₂₃H₂₃N₃O₅; M+H⁺=4.22; M−H⁺=420. ¹H NMR (CD₃OD): δ 7.65 (d, J=8.0, 1H), 7.37 (dd, J=8.2, 0.8, 1H), 7.24-7.04 (m, 8H), 5.87-5.73 (m, 2H), 4.60-4.49 (m, 2H), 4.32-4.23 (m, 1H), 2.69-2.44 (m, 2H), 1.41 (d, J=7.1, 2 sets, 3H).

### EXAMPLE 25

### (3S)-3-[(1-(4′-butenyl)indole-2-carbonyl)valinyl]amino-4-oxobutanoic Acid T-Butyl Ester Semicarbazone

[1-(4′-Butenyl)indole]-2-carboxylic acid (108 mg, 0.5 mmol) and 3(S)-(valinyl)amino-4-oxobutanoic acid, t-butyl ester semicarbazone (163 mg, 0.5 mmol) were dissolved in methylene chloride (3 mL). To this solution was added both DMAP (61 mg, 0.5 mmol) and EDAC (134 mg, 0.7 mmol) under a nitrogen atmosphere at 0° C. and the resultant reaction mixture was stirred for 1 hour at 0° C. and an additional 5 hours at room temperature. The reaction mixture was diluted with ethyl acetate, washed successively with saturated sodium bicarbonate solution and brine, dried under sodium sulfate, and concentrated to give a yellow foam. Trituration of the foam with ether afforded the title product as a slightly yellow powder (146 mg, 55%). TLC: (methanol/methylene chloride: 1/9, silica gel): R_{f}=0.23; ¹H NMR (CDCl₃): δ 8.69 (bs, 1H), 7.64 (d, J=8.0, 1H) 7.41-7.13 (m, 3H), 6.99 (s, 1H), 6.91 (d, J=8.8, 1H), 5.85-5.71 (m, 1H), 5.04-4.94 (m, 3H), 4.65-4.45 (m, 3H), 3.52-2.50 (m, 4H), 2.33-2.26 (m, 1H), 1.41 (s, 9H), 1.05-1.02 (m, 6H).

### EXAMPLE 26

### (3S)-3-[(1-(4′-butenyl]indole-2-carbonyl)valinyl]amino-4-oxobutanoic Acid Semicarbazone

(3S)-3-[(1-(4′-Butenyl)indole-2-carbonyl) valinyl]amino-4-oxobutanoic acid, t-butyl ester semicarbazone (126 mg, 0.24 mmol) was dissolved in anisole (0.2 mL) and methylene chloride (2 mL) and the resulting solution was treated with TFA (1 mL). The acidified reaction mixture was stirred for 4 hours under a nitrogen atmosphere at room temperature. The reaction mixture was concentrated and chased with methylene chloride to give a crude solid. Trituration of the solid with ether afforded the title product as a purple powder (99 mg, 88%). TLC: (methylene chloride:methanol:acetic acid, 20:1:1, silica gel): R_{f}=0.36; ¹H NMR (CD₃OD): δ 8.46 (d, J=8.0, 1H) 8.12 (d, J=8.2, 1H), 7.62 (d, J=8.0, 1H), 7.46 (dd, J=8.5, 0.8, 1H), 7.31-7.21 (m, 2H), 7.31-7.05 (m, 2H), 5.84-5.70 (m, 1H), 4.99-4.78 (m, 3H), 4.62-4.57 (m, 2H), 4.39-4.33 (m, 1H), 2.88-2.69 (m, 2H), 2.52-2.45 (m, 2H), 2.24-2.15 (m, 1H), 1.07-1.02 (m, 6H).

### EXAMPLE 27

### (3S)-3-[(1-(4′-butenyl)indole-2-carbonyl)valinyl]amino-4-oxobutanoic Acid

(3S)-3-[(1-(4′-Butenyl)indole-2-carbonyl) valinyl]amino-4-oxobutanoic acid, semicarbazone (79 mg, 0.17 mmol) was dissolved in methanol (3 mL), formaldehyde (1 mL, 37% wt. aq) and acetic acid (1 mL) and the resulting mixture was stirred for 7 hours under a nitrogen atmosphere at room temperature. The reaction mixture was concentrated in vacuo, diluted with water, and extracted twice with ethyl acetate. The ethyl combined acetate solutions were washed with brine, dried over sodium sulfate and concentrated to give a crude product which was triturated with ether to afford the title product as a light purple powder (24 mg, 34%). TLC: (methylene chloride:methanol:acetic acid, 20:1:1, silica gel): R_{f}=0.60; MS(EI) for C₂₂H₂₇N₃O₅:M+H⁺=414; M−H⁺=412. ¹H NMR (CD₃OD): δ 8.09-8.05 (m, 1H), 7.62 (d, J=8.0, 1H), 7.46 (dd, J=8.5, 0.8, 1H), 7.31-7.25 (m, 1H), 7.13-7.07 (m, 2H), 5.85-5.71 (m, 1H), 4.99-4.90 (m, 3H), 4.62-4.54 (m, 3H), 4.41-4.30 (m, 2H), 2.75-2.46 (m, 4H), 2.22-2.14 (m, 1H), 1.06-1.02 (m, 6H).

### EXAMPLE 28

### (3S)-3-[(1-(2′-(1′-T-butoxy-1′-oxo)ethyl)indole-2-carbonyl)alaninyl]amino-4-oxobutanoic Acid, T-Butyl Ester Semicarbazone

1-[2′-(1′-t-Butoxy-1′-oxo)ethyl]indole-2-carboxylic acid (220 mg, 0.8 mmol) and 3(S)-(alaninyl)amino-4-oxobutanoic acid, t-butyl ester semicarbazone (241 mg, 0.8 mmol) were dissolved in methylene chloride (3 mL) and DMF (1 mL) and the resulting solution was treated with both DMAP (98 mg, 0.8 mmol) and EDAC (211 mg. 1.1 mmol). The resultant reaction mixture was stirred for 1 hour at 0° C. and then an additional 3 hours at room temperature to give a white precipitate. The reaction mixture was concentrated to remove methylene chloride and quenched with 5% KHSO₄ solution. The white solid was collected by filtration, washed with water and ether and dried in vacuo to afford the title product as a white powder (297 mg, 66%). TLC: (methanol/methylene chloride: 1/9, silica gel): R_{f}=0.27. ¹H NMR: (CD₃OD) δ 7.65 (d, J=8.0, 1H), 7.41)d, J=8.0, 1H), 7.26 (s, 1H), 7.22 (d, J=3.0, 1H), 7.16-7.11 (m, 1H), 5.32 (d, J=2.2, 2H), 4.94-4.89 (m, 1H), 4.54 (q, J=7.1, 1H), 2.76 (d, 2H), 1.48 (d, J=7.4, 3H).

### EXAMPLE 29

### (3S)-3-[(1-(carboxymethyl)-indole-2-carbonyl)alaminyl] amino-4-oxobutanoic Acid Semicarbazone

(3S)-3-[(1-(2′-(1′-t-butoxy-1′-oxo)ethyl) indole-2-carbonyl)]amino-4-oxobutanoic acid, t-butyl ester, semicarbazone (274 mg, 0.51 mmol) in methylene chloride (2 mL) was treated with TFA (1 mL). The resulting solution was stirred for 2 hours under a nitrogen atmosphere at room temperature. The reaction mixture was concentrated and chased with methylene chloride to give a solid. Trituration of the solid with ether gave the title product as a light gray powder (262 mg). TLC: (methylene chloride:methanol:acetic acid, 8:1:1, silica gel): R_{f}=0.08. ¹H NMR (CD₃OD): δ 7.65 (d, J=8.0, 1H), 7.41 (d, J=8.0, 1H), 7.26 (s, 1H), 7.22 (d, J=3.0, 1H), 7.16-7.11 (m, 1H), 5.32 (d, J=2.2, 2H), 4.94-4.89 (m, 1H), 4.54 (q, J=7.1, 1H), 2.76 (d, 2H), 1.48 (d, J=7.4, 3H).

### EXAMPLE 30

### (3S)-3-[(1-(carboxymethyl)indole-2-carbonyl)alaninyl]amino-4-oxobutanoic Acid

(3S)-3-[(1-(Carboxymethyl)indole-2-carbonyl) alaninyl]amino-4-oxobutanoic acid, semicarbazone (241 mg, 0.47 mmol) was dissolved in methanol (3 mL), formaldehyde (1 mL, 37% wt. aq) and acetic acid (1 mL) and the resulting solution was stirred for 3 hours under a nitrogen atmosphere at room temperature. The reaction mixture was concentrated in vacuo, diluted with water and extracted twice with ethyl acetate. The combined ethyl acetate solutions were washed with brine, dried under sodium sulfate and concentrated to give a glassy material which was triturated with ether to afford the title product as a slightly yellow powder (114 mg, 63%). TLC: (methylene chloride:methanol:acetic acid, 8:1:1, silica gel): R_{f}=0.16. ¹H NMR (CD₃OD): δ 7.65 (d, J=8.0, 1H), 7.40 (d, J=8.2, 1H), 7.33-7.27 (m, 1H), 7.24 (s, 1H), 7.16-7.10 (m, 1H), 5.36 and 5.26 (AB, J=17.9, 2H), 4.64-4.50 (m, 2H), 4.34-4.20 (m, 1H), 2.72-2.48 (m, 2H), 1.45 (d, J=7.14, 3H, 2 sets).

### EXAMPLE 31

### (3S)-3-[(1-(3′-(1′-T-butoxy-1′-oxo)propyl)indole-2-carbonyl)alaninyl]amino-4-oxobutanoic Acid, T-Butyl Ester Semicarbazone

1-(3′-(1′-t-Butoxy-1′-oxo)propyl)indole-2-carboxylic acid (147 mg, 0.51 mmol) was dissolved in DMF 3 mL) and to the resulting solution was added both DMAP (68 mg, 0.56 mmol) and EDAC (140 mg, 0.73 mmol). Stirring was continued for 10 minutes under a nitrogen atmosphere at 0° C. (3S)-3-(Alaninyl)amino-4-oxobutanoic acid, t-butyl ester semicarbazone (154 mg, 0.51 mmol) was added to the reaction mixture, and the mixture was stirred for 1 hour at 0° C. and then an additional 4 hours at room temperature. The reaction mixture was partitioned between 5% KHSO₄ solution and ethyl acetate. The ethyl acetate solution was washed successively with 5% KHSO₄ solution, saturated sodium bicarbonate solution (2×) and brine, dried over sodium sulfate, and concentrated to give a foam as crude product. Trituration of the foam with ether afforded the title product as a white powder (161 mg, 55%). TLC: (methanol/methylene chloride: 1/9, silica gel): R_{f}=0.36; ¹H NMR (CD₃OD): 7.62 (d, J=8.0, 1H), 7.50 (d, J=8.2, 1H), 7.29 (t, J-8.2, 1H), 7.22 (d, J=3.0, 1H), 7.16 (s, 1H), 7.11 (t, J=7.4, 1H), 4.96-4.90 (m, 1H), 4.82-4.72 (m, 2H), 4.56 (q, J=7.1, 1H), 2.78-2.66 (m, 4H), 1.49 (d, J=7.4, 3H), 1.40 (s, 9H), 1.28 (s, 9H).

### EXAMPLE 32

### (3S)-3-[(1-(2′-carboxyethyl)indole-2-carbonyl)alaninyl]amino-4-oxobutanoic Acid Semicarbazone

(3S)-3-[(1-(3′-(1′-t-Butoxy-1′-oxo)propyl) indole-2-carbonyl)alaninyl]amino-4-oxobutanoic acid, t-butyl ester semicarbazone (140 mg, 0.24 mmol) was dissolved in anisole (0.2 mL) and methylene chloride (2 mL) and the suspension was treated with TFA (1 mL). The resulting solution was stirred for 2 hours under a nitrogen atmosphere at room temperature. The reaction mixture was concentrated and chased with methylene chloride to give a solid. Trituration of the solid with ether gave the title product as a colorless powder (107 mg, 95%). TLC: (methylene chloride:methanol:acetic acid, 8:1:1, silica gel): R_{f}=0.17; ¹H NMR (CD₃OD): δ 7.62 (d, J=8.0, 1H), 7.50 (d, J=8.2, 1H), 7.32-7.27 (m, 1H), 7.23 (d, J=3.0, 1H), 7.13-7.08 (m, 2H), 4.97-4.90 (m, 1H), 4.80-4.69 (m, 1H), 4.54 (q, J=7.1, 1H), 2.82-2.73 (m, 4H), 1.49 (d, J=7.1, 3H).

### EXAMPLE 33

### (3S)-3-[(1-(2′-carboxyethyl)indole-2-carbonyl)alaninyl]amino-4-oxobutanoic Acid

(3S)-3-[(1-(2′-Carboxyethyl)indole-2-carbonyl) alaninyl]amino-4-oxobutanoic acid, semicarbazone (95 mg, 0.21 mmol) was dissolved in methanol (3 mL), formaldehyde (1 mL, 37% wt. aq) and acetic acid (1 mL) and the resultant solution was stirred for 4 hours under a nitrogen atmosphere at room temperature. The reaction mixture was concentrated to remove methanol, diluted with water and extracted twice with ethyl acetate. The combined ethyl acetate solutions were washed with brine, dried over sodium sulfate and concentrated to give a glassy material which was triturated with ether to afford the title product as a slightly yellow powder (20 mg, 20%). TLC: (methylene chloride:methanol:acetic acid, 8:1:1, silica gel): R_{f}=0.26; ¹H NMR (CD₃OD): δ 7.62 (d, J=8.0, 1H), 7.51 (d, J=1H), 7.32-7.27 (m, 1H), 7.13-7.08 (m, 2H), 4.80-4.76 (m, 2H), 4.68-4.52 (m, 2H), 4.37-4.25 (m, 1H), 2.84-2.50 (m, 3H), 1.47 (d, J=7.1, 3H, 2 sets).

### EXAMPLE 34

### 2,6-dichlorobenzyloxyethanol

Sodium hydride (1.76 g, 0.044 mol, 60% wt. in mineral oil) was slowly added to a solution of ethylene glycol (11.2 mL) in dry THF (100 mL). The resultant mixture was stirred briefly under a nitrogen atmosphere at room temperature. α-Bromo-2,6-dichlorotoluene (9.894 g, 0.04 mol) was added to the mixture and the mixture was stirred for an additional 5.5 hours under a nitrogen atmosphere at room temperature. Additional sodium hydride (0.400 g) was added and the mixture was then stirred for 24 hours at room temperature. The reaction mixture was concentrated to remove THF, and the residue was partitioned between ether and water. The aqueous layer was back extracted with ether (2×). The combined organic solution was washed with water and brine, dried over sodium sulfate, filtered and concentrated to give a crude oil. The oil was flash chromatographed on silica gel with ethyl acetate/hexanes (10-50%) to give the title product as a yellow oil (4.56 g, 51%). TLC: (ethyl acetate/hexanes, 30/70): R_{f}=0.26. ¹H NMR (CDCl₃): δ 7.35-7.18 (m, 3H), 4.84 (s, 2H), 3.76-3.66 (m, 4H).

### EXAMPLE 35

### 5-(2′,6′-dichlorobenzyloxy)-4-hydroxy-3-nitropentanoic Acid T-Butyl Ester

DMSO was added dropwise to a solution of (47.5 mL) oxalyl chloride (7.5 mL, 15.0 mmol, 2.0 M in methylene chloride) and the resultant reaction mixture was stirred for 10 min at −78° C. 2,6-Dichlorobenzyloxy-ethanol (2211 mg, 10 mmol)in dry methylene chloride (5 mL) was added dropwise to the mixture and the mixture was then stirred for 15 minutes under a nitrogen atmosphere at −78° C. Triethylamine (8.4 mL, 60 mmol) was added dropwise to the reaction mixture, and the resultant mixture was stirred for 10 min at −78° C., then allowed to warm to 0° C. (over a period of approximately 20 min). A methylene chloride solution of tent-butyl 3-nitropropionate (1927 mg, 11.0 mmol in 5 mL of dry methylene chloride) was added dropwise to the reaction mixture and the mixture was stirred for 1 hour. The residue was extracted with ether and the resultant white solid was collected by filtration. The organic filtrate was washed with 5% KHSO₄ solution (2×) and brine, dried over sodium sulfate, and concentrated to give a crude oil (3.95 g). The oil was subjected to flash chromatography on silica gel with ethyl acetate/hexanes (1:2) to afford the title product as a yellow oil (2.917 g, 74%). TLC: (ethyl acetate, hexanes, 60/40): R_{f}=0.54.

### EXAMPLE 36

### 3-amino-5-(2′,6′-dichlorobenzyloxy)-4-hydroxypentanoic Acid T-Butyl Ester

A mixture of 5-(2′,6′-dichlorobenzyloxy)-4-hydroxy-3-nitropentanoic acid t-butyl ester (2.213 g, 0.0056 mol) and wet Raney nickel (3.4 g) in methanol (150 mL) was stirred for 2 hours under a hydrogen balloon at room temperature. The reaction mixture was filtered through Celite and the filter cake was washed with methanol. The filtrate was concentrated and chased with methylene chloride to give the title product (2.078 g, 100%). TLC: (methanol/methylene chloride 1/9): R_{f}=0.21.

### EXAMPLE 37

### N-(1,3-dimethylindole-2-carbonyl)valine

DMAP (367 mg, 3.0 mmol) and EDAC (748 mg, 3.9 mmol) were added as solids to a solution of 1,3-dimethylindole-2-carboxylic acid (568 mg, 3.3 mmol) in DMF (5 mL), and the resultant mixture was stirred for 10 minutes under a nitrogen atmosphere at 0° C. A methylene chloride solution of the methyl ester of valine (553 mg, 3.3 mmol, in 5 mL of methylene chloride) was added to the mixture, and the mixture was first stirred for one hour at 0° C. then for 5 hours at room temperature. The reaction mixture was partitioned between ethyl acetate and 5% KHSO₄ solution and the aqueous solution was back-extracted with ethyl acetate. The combined ethyl acetate washes were in turn washed with 5% KHSO₄ solution saturated sodium bicarbonate solution (2×) and brine, dried over sodium sulfate, and concentrated to give the title product as a yellow syrup (900 mg).

A 1,4-dioxane solution (5 mL) of the above yellow syrup was treated with an aqueous solution of lithium hydroxide (1.0 M LiOH, 3.0 mL) and the resultant mixture was stirred for 1 hour at room temperature (the mixture became homogeneous). The reaction mixture was acidified with 1 M hydrochloric acid and extracted with ethyl acetate (3×). The combined ethyl acetate solutions were leashed with brine, dried over sodium sulfate, and concentrated to give the title product as a yellow foam (839 mg). ¹H NMR (CD₃OD): δ 7.58 (dt, J=8.0, 0.8, 1H), 7.37 (dd, J-8.0, 0.8, 1H), 7.29-7.24 (m, 1H), 7.12-7.06 (m, 1H), 4.57 (d, J=5.8, 1H), 3.80 (s, 3H), 2.48 (s, 3H), 3.34-2.28 (m, 1H), 1.10 (d, J=6.9, 3H), 1.07 (d, J=6.9, 3H).

### EXAMPLE 38

N-[(1,3-dimethylindole-2-carbonyl)valinyl]-3-amino-4-hydroxy-5-(2′,6′-dichlorobenzyloxy)pentanoic Acid, T-Butyl Ester

1-Hydroxybenzotriazole hydrate (153 mg, 1.0 mmol) and EDAC (268 mg, 1.4 mmol) were added to a methylene chloride solution of N-(1,3-dimethylindole-2-carbonyl)valine (288 mg, 1.0 mmol, in 3 mL of methylene chloride). The resultant mixture was stirred for 10 minutes under a nitrogen atmosphere at room temperature. A methylene chloride solution of 3-amino-5-(2′,6′-dichlorobenzyloxy)-4-hydroxypentanoic acid, t-butyl ester (364 mg, 1.0 mmol, in 2 mL of methylene chloride) was added to the reaction mixture and the mixture was first stirred for one hour under a nitrogen atmosphere at 0° C., and then for 16 hours at room temperature. The reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×) and brine, dried over sodium sulfate, and concentrated to give crude product (583 mg). The crude product was subjected to flash chromatography on silica gel with ethyl acetate/hexane (2/3) to give the title product as a white solid (260 mg). TLC: (ethyl acetate/hexanes 1:1): R_{f}=0.38.

### EXAMPLE 39

N-[(1,3-dimethylindole-2-carbonyl)valinyl]-3-amino-4-oxo-5-(2′6′-dichlorobenzyloxy)pentanoic Acid, T-Butyl Ester

Dess-Martin periodinane (195 mg) was added as a solid to a solution of N-[(1,3-dimethylindole-2-carbonyl)valinyl]-3-amino-4-hydroxy-5-(2′,6′-dichlorobenzyloxy)pentanoic acid, t-butyl ester (96 mg) in DMSO (1.5 ml). The resulting solution was stirred under a nitrogen atmosphere at room temperature for thirty minutes, then partitioned between EtOAc and water. The organic phase was washed with water (2×) and brine, dried (Na₂SO₄), and concentrated to give a white solid (83 mg). Flash chromotographic purification with EtOAc/hexanes (1:1) afforded the title product as a white solid (54 mg). TLC (EtOAc/hexanes; 1:1, silica gel): R_{f}=0.52.

### EXAMPLE 40

N-[(1,3-dimethylindole-2-carbonyl)valinyl]-3-amino-4-oxo-5-(2′6′-dichlorobenzyloxy)Pentanoic Acid

A solution of N-[(1,3-Dimethylindole-2-carbonyl)valinyl]-3-amino-4-oxo-5-(2′,6′-dichloro-benzyloxy)pentanoic acid, t-butyl ester (49 mg) in anisole (0.2 mL) and methylene chloride (2 mL) was treated with TFA (1 mL) and stirred for 30 minutes under a nitrogen atmosphere at room temperature. The resultant solution was concentrated and chased with methylene chloride to give a white solid as the crude product. The crude product was triturated with ether to yield the title product as a white powder (34 mg). MS(EI) for C₂₈H₃₁C1₂N₃O₆; MH⁺=576/578; (MH)=574/576.

### EXAMPLE 41

N-[(1,3-dimethylindole-2-carbonyl)valinyl]-3-amino-4-hydroxy-5-fluoropentanoic Acid, T-Butyl Ester

4-Dimethylaminopyridine (DMAP) (67 mg, 0.55 mmol) and 1-(3′-dimethylaminopropyl)-3-ethylcarbodiimide hydrochloride (EDAC) (125 mg, 0.65 mmol) were added as solids to a DMF solution of 1,3-dimethylindole-2-carboxylic acid (95 mg, 0.5 mmol in 1 ML of DMF), and the resultant reaction mixture was stirred for 10 minutes under a nitrogen atmosphere at 0° C. A methylene chloride solution of N-(valinyl)-3-amino-4-hydroxy-5-fluoropentanoic acid, t-butyl ester (153 mg, 0.5 mmol in 1 mL of methylene chloride) was added and the resultant reaction mixture was first stirred for 1 hour at 0° C. and then for 4 hours at room temperature. The reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×), and brine, dried over sodium sulfate, and concentrated to give a solid. The solid was triturated with ether/hexane to yield the title product as a white solid (134 mg, 56%). TLC: (ethyl acetate/hexanes, 2:1): R_{f}=0.42. ¹H NMR (CDCl₃): δ 7.59 (d, J=8.8, 1H), 7.37 (d, J=7.7, 1H), 7.29-7.24 (m, 1H), 7.12-7.07 (m, 1H), 4.49-4.26 (m, 5H), 3.81-3.79 (m, 3H), 2.66-2.47 (m, 5H), 2.22-2.10 (m, 1H), 1.45-1.41 (m, 9H), 1.09-1.03 (m, 6H).

### EXAMPLE 42

N-[(1,3-dimethylindole-2-carbonyl)valinyl]-3-amino-4-oxo-5-fluoropentanoic Acid, T-Butyl Ester

Dimethyl sulfoxide (0.09 mL, 1.25 mmol) was added to a solution of oxalyl chloride (0.19 mL, 2.0 M, 0.38 mmol) in methylene chloride (4 mL), and the resultant mixture was stirred for 10 minutes under a nitrogen atmosphere at −78° C. A dry methylene chloride solution of N-[1,3-dimethylindole-2-carbonyl)valinyl]-3-amino-4-hydroxy-5-fluoropentanoic acid, t-butyl ester (119 mg, 0.25 mmol in 1 mL of dry methylene chloride), was added dropwise to the mixture and the resultant reaction mixture was stirred for 15 min at −78° C. Triethylamine (0.21 mL, 1.5 mmol) was added dropwise, and the reaction mixture was then stirred for 10 minutes at −78° C., then was allowed to warm to room temperature. The reaction mixture was partitioned between ethyl acetate and 5% KHSO₄ solution and the aqueous layer was back-extracted with ethyl acetate. The combined ethyl acetate solutions were washed with 5% KHSO₄ solution and brine, dried over sodium sulfate, and concentrated to give a crude product. The crude product was chromatographed with ethyl acetate/hexanes (2:1) on silica gel gave the title product as a white solid (48 mg, 41%). TLC: (ethyl acetate/hexanes, 2:1): R_{f}=0.58.

### EXAMPLE 43

N-[(1.3-dimethylindole-2-carbonyl)valinyl]-3-amino-4-oxo-5-fluoropentanoic Acid

A solution of N-[(1,3-dimethylindole-2-carbonyl)valinyl]-3-amino-4-oxo-5-fluoropentanoic acid, t-butyl ester (40 mg) in anisole (0.2 ml) and methylene chloride (2 mL) was treated with trifluoroacetic acid (1 mL), and the resultant reaction mixture was stirred for 30 minutes under a nitrogen atmosphere at room temperature. The reaction mixture was concentrated and chased with methylene chloride to give a solid. The solid was triturated with ether to yield the title product as a brown powder (17 mg). TLC: (methylene chloride/methanol/acetic acid, 20:1:1): R_{f}=0.40. MS (El) for C₂₁H₂₆FN₃O₅: MH⁺=4.20; MH⁻=418.

### EXAMPLE 44

### N-[(1-methylindole-2-carbonyl)valinyl]-3-amino-4-hydroxy-5-fluoropentanoic Acid T-Butyl Ester

DMAP (95 mg, 0.78 mmol) and EDAC (200 mg, 1.04 mmol) were added as solid to a solution of 1-methylindole-2-carboxylic acid (130 mg, 0.74 mmol) and N-(valinyl)-3-amino-4-hydroxy-5-fluoropentanoic acid, t-butyl ester (227 mg, 0.74 mmol) in methylene chloride (5 mL), and the resultant solution was stirred for 1 hour under a nitrogen atmosphere at 0° C. and then 4 hours at room temperature. The reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×) and brine, dried over sodium sulfate, and concentrated to give a foam. The foam was triturated with ether to yield the title product as a slightly brown solid (224 mg, 65%). TLC: (methanol/methylene chloride, 1:9): R_{f}=0.46.

### EXAMPLE 45

### N-[(3-chloro-L-methylindole-2-carbonyl)valinyl]-3-amino-4-oxo-5-fluoropentanoic Acid, T-Butyl Ester

DMSO (0.06 mL, 0.9 mmol) was added to a solution of oxalyl chloride (0.14 mL, 2.0 M, 0.28 mmol, in 4 mL of methylene chloride) and the solution was then stirred for 10 minutes under a nitrogen atmosphere at −78° C. A solution of N-[(1-methylindole-2-carbonyl) valinyl]-3-amino-4-hydroxy-5-fluoropentanoic acid, t-butyl ester (85 mg, 0.18 mmol) in dry methylene chloride (1 mL), was added dropwise to the reaction mixture and the mixture was stirred for 15 minutes at −78° C. Triethylamine (0.15 mL, 1.08 mmol) was added dropwise to the reaction mixture and the mixture was stirred for 10 minutes at −78° C. and then was allowed to warm to room temperature. The reaction mixture was partitioned between ethyl acetate and 5% KHSO₄ solution and the aqueous layer was back-extracted with ethyl acetate.

The combined ethyl acetate solutions were washed with 5% KHSO₄ solution and brine, dried over sodium sulfate, and concentrated to give a brown foam. The foam was triturated with ether to afford the title product as a light brown powder (64 mg). MS for C₂₄H₃₁C1FN₃O₅: (MH)⁻=494/496.

### EXAMPLE 46

### N-[(3-chloro-1-methylindole-2-carbonyl)valinyl]-3-amino-4-oxo-5-fluoropentanoic Acid

A solution N-[(3-chloro-1-methylindole-2-carbonyl)valinyl]-3-amino-4-oxo-5-fluoropentanoic acid, t-butyl ester (47 mg) in anisole (0.2 mL) and methylene chloride (2 mL) was treated with TFA (1 mL) and the resultant reaction mixture was stirred for 1 hour under a nitrogen atmosphere at room temperature. The reaction mixture was concentrated and chased with methylene chloride, then triturated with ether to afford a brown powder (28 mg). The powder was subjected to flash chromatography on silica gel with methanol/methylene chloride containing a drop of acetic acid to give the title product (25 mg). TLC: (methylene chloride/methanol, 9:1): R_{f}=0.29. MS(EI) for C₂₀H₂₃C1FN₃O₅: MH⁺=440.442; (M−H)⁻=438/440.

### EXAMPLE 47

### N-[(5-fluoro-1-methylindole-2-carbonyl)valinyl]-3-amino-4-hydroxy-5-fluoropentanoic Acid, T-Butyl Ester

DMAP (257 mg, 2.08 mmol) and EDAC (427 mg, 2.23 mmol) were added as solids to a solution of 5-fluoro-1-methylindole-2-carboxylic acid (359 mg, 86 mmol in 3 mL of DMF), and the resultant reaction mixture was stirred for 10 minutes under a nitrogen atmosphere at 0° C. N-(Valinyl)-3-amino-4-hydroxy-5-fluoropentanoic acid, t-butyl ester (579 mg, 1.86 mmol) in DMF (3 mL) was added and the resulting solution was stirred for 1 hour under a nitrogen atmosphere at 0° C. and 4 hours at room temperature. The reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×) and brine, dried over sodium sulfate, and concentrated to give the title product as a slightly yellow solid (0.827 mg). TLC: (methanol/methylene chloride, 1:9): R_{f}=0.52.

### EXAMPLE 48

### N-[(3-chloro-5-fluoro-1-methylindole-2-carbonyl)valinyl]-3-amino-4-oxo-5-fluoropentanoic Acid, T-Butyl Ester

DMSO (0.60 mL, 8.5 mmol) was added to a methylene chloride solution of oxalyl chloride (2.1 mL, 2.0 M, 4.2 mmol, in 15 mL of methylene chloride), and the resultant reaction mixture was stirred for 10 minutes under a nitrogen atmosphere at −78° C. A methylene chloride solution of N-[(5-fluoro-1-methylindole-2-carbonyl)valinyl]-3-amino-4-hydroxy-5-fluoropentanoic acid, t-butyl ester (820 mg, 1.7 mmol, in 8 mL of dry methylene chloride), and DMSO (0.4 mL) were added dropwise to the reaction mixture and stirred for 15 minutes at −78° C. TEA (1.4 mL, 10.2 mmol) was added to the mixture dropwise and the mixture was stirred for 10 minutes at −78° C., then was allowed to warm to room temperature. The reaction mixture was partitioned between ethyl acetate and 5% KHSO₄ solution and the aqueous layer was back-extracted with ethyl acetate. The combined ethyl acetate solutions were washed with 5% KHSO₄ solution and brine, dried over sodium sulfate, and concentrated to give the title product as a slightly yellow solid. Trituration with ether afforded the title product as a white powder (705 mg, 85%). TLC: (methanol/methylene chloride, 1:9): R_{f}=0.63. MS for C₂₄H₃₀C1F₂N₃O₅: MH⁺=514/516; (M−H)⁻=512/514.

### EXAMPLE 49

### N-[(3-chloro-5-fluoro-1-methylindole-2-carbonyl)valinyl]-3-amino-4-oxo-5-fluoropentanoic Acid

A solution of N-[(3-chloro-5-fluoro-1-methylindole-2-carbonyl)valinyl]-3-amino-4-oxo-5-fluoropentanoic acid, t-butyl ester (682 mg) in anisole (1 mL) and methylene chloride (10 mL) was treated with TFA (5 mL), and the resultant reaction mixture was stirred for 45 minutes under a nitrogen atmosphere at room temperature. The reaction mixture was concentrated and chased with methylene chloride, then triturated with ether to afford the title product as a white powder (500 mg). MS (EI) for C₂₀H₂₂C1F₂N₃O₅: MH⁺=458/460; (M−H)⁻=456/458.

### EXAMPLE 50

### N-[(1-(3′-phenylpropyl)indole-2-carbonyl)valinyl]-3-amino-4-hydroxy-5-fluoropentanoic Acid, T-Butyl Ester

DMAP (122 mg, 1.0 mmol) and EDAC (249 mg 1.3 mmol) were added as solids to a DMF solution of 1-(3′-phenylpropyl)indole-2-carboxylic acid (279 mg, 1.0 mmol, 2 mL in DMF), and the resultant mixture was stirred for 10 minutes under a nitrogen atmosphere at 0° C. A methylene chloride solution of N-(valinyl)-3-amino-4-hydroxy-5-fluoropentanoic acid, t-butyl ester (306 mg, 1.0 mmol in 2 mL of methylene chloride) was added to the reaction mixture and the mixture was stirred for 1 hour under a nitrogen atmosphere at 0° C. and then 4 hours at room temperature. The yellow reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×) and brine, dried over sodium sulfate, and concentrated to give a crude solid (0.827 g). The crude solid was subjected to flash chromatography on silica gel eluting with ethyl acetate/hexanes (1:2) afforded the title product as a slightly yellow solid (171 mg). TLC: (ethyl acetate/hexanes 2:1): R_{f}=0.57.

### EXAMPLE 51

### N-[(1-(3′-phenylpropyl)indole-2-carbonyl)valinyl]-3-amino-4-oxo-5-fluoropentanoic Acid, T-Butyl Ester

DMSO (0.11 mL, 1.5 mmol) was added to a methylene chloride solution of oxalyl chloride (0.22 mL, 2.0 M, 0.44 mmol in 3.5 mL in methylene chloride), and the resultant solution was stirred for 10 minutes under a nitrogen atmosphere at −78° C. A methylene chloride solution of N-[(1-(3′-phenylpropyl)indole-2-carbonyl)valinyl]-3-amino-4-hydroxy-5-fluoropentanoic acid, t-butyl ester (169 mg, 0.3 mmol in 1.5 mL of dry methylene chloride) was added dropwise and the resulting solution stirred for 15 minutes at −78° C. Triethylamine (0.25 mL, 1.8 mmol) was added dropwise to the reaction mixture and the mixture was stirred for 10 minutes at −78° C., then was allowed to warm to room temperature. The reaction mixture was partitioned between ethyl acetate and 5% KHSO₄ solution and the aqueous layer was back-extracted with ethyl acetate. The combined ethyl acetate solutions were washed with 5% KHSO₄ solution and brine, dried over sodium sulfate, and concentrated to give a crude product. The crude product was triturated with hexanes to yield the title product as a slightly yellow powder (129 mg, 77%). TLC: (ethyl acetate/hexanes 2:1): R_{f}=0.69.

### EXAMPLE 52

### N-[(1-(3′-phenylpropyl)indole-2-carbonyl)valinyl]-3-amino-4-oxo-5-fluoropentanoic Acid

A solution of N-[(1-(3′-phenylpropyl)indole-2-carbonyl)valinyl]-3-amino-4-oxo-5-fluoropentanoic acid, t-butyl ester (97 mg) in anisole (0.2 mL) and methylene chloride (2 mL) was treated with TFA (1 mL), and the resultant reaction mixture was stirred for 1 hour under a nitrogen atmosphere at room temperature. The reaction mixture was concentrated and chased with methylene chloride, then triturated with ether to yield the title product as a slightly yellow powder (44 mg). TLC: (methylene chloride/methanol/acetic acid, 20:1:1): R_{f}=0.4; MS (EI) for C₃₂H₄₀FN₃O₅: MH⁺=510; (M−H)⁻=508.

### EXAMPLE 53

N-[(1-phenylindole-2-carbonyl)valinyl]-3-amino-4-hydroxy -5-fluoropentanoic Acid, T-Butyl Ester

DMAP (122 mg, 1.0 mmol) and EDAC (249 mg, 1.3 mmol) were added as solids to a DMF solution of 1-phenylindole-2-carboxylic acid (237 mg, 1.0 mmol in 2 mL DMF), and the resultant reaction mixture was stirred for 10 minutes under a nitrogen atmosphere at 0° C. A methylene chloride solution of N-(valinyl)-3-amino-4-hydroxy-5-fluoropentanoic acid, t-butyl ester (306 mg, 1.0 mmol in 2 mL of methylene chloride) was added to the reaction mixture and the mixture was stirred for 1 hour under a nitrogen atmosphere at 0° C. and 4 hours at room temperature. The yellow reaction mixture was partitioned between ethyl acetate and 5% KHSO₄ solution and the aqueous solution was back-extracted with ethyl acetate. The combined ethyl acetate solutions were washed with 5% KHSO₄ solution, saturated sodium bicarbonate solution (2×) and brine, dried over sodium sulfate, and concentrated to give a colorless film (0.827 g). The film was subjected to flash chromatography on silica gel with ethyl acetate/hexanes (1:2) to yield the title product as a white foam (400 mg, 78%). TLC: (ethyl acetate/hexanes 1:1): R_{f}=0.27.

### EXAMPLE 54

N-[(1-phenylindole-2-carbonyl)valinyl]-3-amino-4-oxo-5-fluoropentanoic Acid, T-Butyl Ester

DMSO (0.13 mL, 1.9 mmol) was added to a methylene chloride solution of oxalyl chloride (0.29 mL, 2.0 M, 0.58 mmol in 4 mL of methylene chloride), and the resultant solution was stirred for 10 minutes under a nitrogen atmosphere at 78° C. A methylene chloride solution of N-[(1-phenylindole-2-carbonyl)valinyl]-3-amino-4-hydroxy-5-fluoropentanoic acid, t-butyl ester (200 mg, 0.38 mmol in 2 mL of dry methylene chloride) was added dropwise and resulting mixture stirred for 15 minutes at −78° C. Triethylamine (0.30 mL, 2.1 mmol) was added dropwise to the mixture, and the resultant mixture was stirred for 10 minutes at −78° C., then was allowed to warm to room temperature. The reaction mixture was partitioned between ethyl acetate and 5% KHSO₄ solution and the aqueous layer was back-extracted with ethyl acetate. The combined ethyl acetate solutions were washed with 5% KHSO₄ solution and brine, dried over sodium sulfate, and concentrated to give a crude product. The crude product was triturated to yield the title product as a slightly yellow powder (181 mg). TLC: (ethyl acetate/hexanes 1:1): R_{f}=0.43.

### EXAMPLE 55

### N-[(1-phenylindole-2-carbonyl)valinyl]-3-amino-4-oxo-5-fluoropentanoic Acid

A solution of N-[(1-phenylindole-2-carbonyl)valinyl]-3-amino-4-oxo-5-fluoropentanoic acid, t-butyl ester (154 mg) in anisole (0.2 mL) and methylene chloride (2 mL) was treated with TFA (1 mL), and the resultant reaction mixture was stirred for one hour under a nitrogen atmosphere at room temperature. The reaction mixture was concentrated and chased with methylene chloride, then triturated with ether to yield the title product as a white powder (100 mg). TLC: (methylene chloride/methanol/acetic acid, 20:1:1): R_{f}=0.38, MS (EI) for C₂₅H₂₆FN₃O₅: MH⁺=468; (M−H)⁻=466.

### EXAMPLE 56

### N-[1-(2′-((1′-T-butoxy-1′-oxo)ethyl)indole-2-carbonyl) valinyl]-3-amino-4-hydroxy-5-fluoropentanoic Acid, T-Butyl Ester

DMAP (122 mg, 1.0 mmol) and EDAC (249 mg, 1.3 mmol) were added as solids to a DMF solution of (1-(2′-((1′-t-butoxy-1′-oxo)ethyl)indole-2-carboxylic acid (275 mg, 1.0 mmol in 2 mL of DMF), and the resultant solution was stirred for 10 minutes under a nitrogen atmosphere at 0° C. A methylene chloride solution of N-(valinyl)-3-amino-4-hydroxy-5-fluoropentanoic acid, t-butyl ester (306 mg, 1.0 mmol in 2 mL of methylene chloride) was added to it, stirred for 1 hour under a nitrogen atmosphere at 0° C. and 4 hours at room temperature. The yellow reaction mixture was partitioned between ethyl acetate and 5% KHSO₄ solution and the aqueous solution was back-extracted with ethyl acetate. The combined ethyl acetate solutions were washed with 5% KHSO₄ solution, saturated with sodium bicarbonate solution (2×) and brine, dried over sodium sulfate, and concentrated to give a colorless film (0.827 g). The film was flash chromatographed on silica gel with ethyl acetate/hexane (1:1) to yield the title product as a white foam (461 mg). TLC: (ethyl acetate/hexanes 30:70): R_{f}=0.11.

### EXAMPLE 57

N-[(1-(2′-((1′-T-butoxy-1′-oxo)ethyl)indole-2-carbonyl) valinyl]-3-amino-4-oxo-5-fluoropentanoic Acid, T-Butyl Ester

A mixture of N-[(1-(2′-((1′-t-butoxy-1′-oxo)ethyl)indole-2-carbonyl)valinyl]-3-amino-4-hydroxy-5-fluoropentanic acid, t-butyl ester (230 mg, 0.41 mmol), N-methylmorpholine N-oxide (71 mg, 0.61 mmol) and powdered molecular sieves (205 mg) in dry methylene chloride (2 mL) was stirred for 1.5 hours under a nitrogen atmosphere at room temperature. Tetra(propyl)ammonium perruthenate (7 mg) was added and the resulting mixture was stirred for 2 hours under a nitrogen atmosphere at room temperature. The reaction mixture was filtered through silica gel with ethyl acetate as the eluent. The filtrate was concentrated and chromatographed on silica gel with ethyl acetate/hexanes (approximately 1:2 to approximately 1:1) to yield the title product as a yellow oil (100 mg). TLC: (ethyl acetate/hexanes 30/70): R_{f}=0.27.

### EXAMPLE 58

### N-[(1-(carboxymethyl)indole-2-carbonyl)valinyl]-3-amino-4-oxo-5-fluoropentanoic Acid

A solution of N-[(1-(2′-((1′-t-butoxy-1′-oxo)ethyl)indole-2-carbonyl)valinyl]-3-amino-4-oxo-5-fluoropentanoic acid, t-butyl ester (100 mg) in anisole (0.2 mL) and methylene chloride (2 mL) was treated with TFA (1 mL). The resultant reaction mixture was stirred for 30 minutes under a nitrogen atmosphere at room temperature. The reaction mixture was concentrated and chased with methylene chloride, then triturated with ether to yield the title product as a light yellow powder (26 mg). TLC: (methylene chloride/methanol, 8:1:1): R_{f}=0.32. MS (EI) for C₂₁H₂₄FN₃O₇: MH⁺=450; (M−H)⁻=448.

### EXAMPLE 59

N-[(1-methylindole-2-carbonyl)valinyl]-3-amino-4-hydroxy-5-fluoropentanoic Acid, T-Butyl Ester

To a solution of 1-methylindole-2-carboxylic acid (130 mg, 0.74 mmol) and N-(valinyl)-3-amino-4-hydroxy-5-fluoropentanoic acid, tert-butyl ester in methylene chloride (5 mL) and cooled to 0° C. Solid 4-dimethylaminopyridine (DMAP) (95 mg, 0.78 mmol) and 1-(3′-dimethylaminopropyl)-3-ethylcarbodiimide hydrochloride (EDAC) (200 mg, 1.04 mmol) were added to the solution at 0° C. The reaction mixture was stirred at 0° C. for 1 h and allowed to warm slowly to room temperature. After 4 h the reaction was partitioned between ethyl acetate (EtOAc) and 5% KHSO₄ aqueous solution. The organic layer was washed with 5% KHSO₄ solution, saturated sodium bicarbonate solution, brine, dried (Na₂SO₄) and concentrated to a foam. The crude residue was triturated with diethyl ether and the solid filtered to afford the title compound as a light brown solid (224 mg, 65% yield). TLC(MeOH:CH₂Cl₂, 1:9): R_{f}=0.46.

### EXAMPLE 60

N-[(1-methylindole-2-carbonyl)valinyl]-3-amino-4-oxo-5-fluoropentanoic Acid, T-Butyl Ester

To a solution of N-[(1-methylindole-2-carbonyl)valinyl]-3-amino-4-hydroxy-5-fluoropentanoic acid, t-butyl ester (51 mg, 0.11 mmol) in DMSO(1 mL) was added Dess-Martin periodinane (110 mg). After 30 min at room temperature the reaction mixture was partitioned between ethyl acetate and water. The organic layer was washed with water and brine, dried and concentrated to a white solid. Trituration with diethyl ether and collection of the solid afforded the title compound as a white powder (25 mg, 49% yield). TLC(MeOH:CH₂Cl₂, 5:95): R_{f}=0.48.

### EXAMPLE 61

### N-[(1-methylindole-2-carbonyl)valinyl]-3-amino-4-oxo-5-fluoropentanoic Acid

A solution of N-[(1-methylindole-2-carbonyl)valinyl]-3-amino-4-oxo-5-fluoropentanoic acid, t-butyl ester (19 mg, 0.041 mmol) and anisole (0.1 mL) in CH₂Cl₂ (1 mL) was treated with trifluoroacetic acid (0.5 mL) at room temperature. After 30 min the reaction mixture was concentrated and chased with methylene chloride. The crude residue was triturated with diethyl ether and the solid filtered to afford the title compound as a light brown solid (12 mg, 72% yield). TLC(AcOH:MeOH:CH₂Cl₂, 1:1:20): R_{f}=0.59. Mass Spectrum for C₂₀H₂₄FN₃O₅: [MH]⁺406, [MH]⁻404.

Following the methods set down in Examples 59-61, the following compounds were prepared:

### EXAMPLE 62

### N-[(1.3-dimethyl-5-fluoroindole-2-carbonyl)valinyl]-3-amino-4-oxo-5-fluoropentanoic Acid

57% yield; TLC(MeOH:CH₂Cl₂, 5:95): R_{f}=0.56. Mass Spectrum for C₂₁H₂₅F₂N₃O₃: [MH]⁺438, [MH]⁻436.

### EXAMPLE 63

### N-[(1-homoallylindole-2-carbonyl)valinyl]-3-amino-4-oxo-5-fluoropentanoic Acid

29% yield; TLC(MeOH:CH₂Cl₂, 1:9): R_{f}=0.33. Mass Spectrum for C₂₃H₂₈FN₃O₅: [MH]⁺446, [MNa]⁺468, [MH]⁻444.

### EXAMPLE 64

N-[(1-methyl-5-fluoroindole-2-carbonyl)valinyl]-3-amino-4-oxo-5-fluoropentanoic Acid

89% yield; TLC(MeOH:CH₂Cl₂,9:1): R_{f}=0.14. Mass Spectrum for C₂₀H₂₃F₂N₃O₅: [MH]⁺424, [MH]⁻422.

### EXAMPLE 65

### N-[(1-methyl-3-isobutylindole-2-carbonyl)valinyl]-3-amino-4-oxo-5-fluoropentanoic Acid

50% yield; TLC(MeOH:CH₂Cl₂,9:1): R_{f}=0.20. Mass Spectrum for C₂₄H₃₂FN₃O₅: [MH]⁺462, [MH]⁻460.

### EXAMPLE 66

N-[(1-methyl-3-phenethylindole-2-carbonyl)valinyl]-3-amino-4-oxo-5-fluoropentanoic Acid

38% yield; TLC(ethyl acetate: hexanes, 1:1): R_{f}=0.19. Mass Spectrum for C₂₈H₃₂FN₃O₅: [MH]⁺510, [MH]⁻508.

### EXAMPLE 67

### N-[(1-methyl-5-obenzylindole-2-carbonyl)valinyl]-3-amino-4-oxo-5-fluoropentanoic Acid

78% yield; TLC(ethyl acetate: hexanes, 1:1): R_{f}=0.17. Mass Spectrum for C₂₇H₃₀FN₃O₆: [MH]⁺512, [MH]⁻510.

### EXAMPLE 68

### N-(1,3-dimethyl-indole-2-carbonyl)-valinyl-3-amino-5-bromo-4-oxo-pentanoic Acid, T-Butyl Ester

1-Hydroxybenzotriazole hydrate (3.19 g, 20.8 mmol) and 1-(3′-dimethylaminopropyl)-3-ethylcarbodiimide hydrochloride (EDAC) (5.60 g, 29.2 mmol) were added to a stirred solution of N-carbobenzyloxycarbonyl valine (5.24 g, 20.8 mmol) in methylene chloride/dimethyl formamide (DMF) (60 ml/30 ml) at 0° C. under nitrogen. After 15 min, aspartic acid α-methyl, β-tert-butyl diester (5.00 g, 20.8 mmol) was added as a solid followed by neat 4-methylmorpholine (2.40 ml, 21.8 mmol). After stirring at 0° C. for 1 hour and at room temperature for 5 hours, the mixture was partitioned between ethyl acetate and 5% KHSO₄ solution. The aqueous solution was back-extracted with ethyl acetate and the combined extracts were washed with saturated NaHCO₃ and brine, dried over sodium sulfate, and concentrated to give a solid. Trituration with ether afforded of N-[carbobenzyloxycarbonyl valinyl]aspartic acid, α-methyl, β-tert-butyl diester as a white solid (8.36 g, 92%). TLC(CH₂Cl₂/MeOH, 95/5): R_{f}=0.48.

A solution of the above product (4.00 g, 9.17 mmol) in 200 ml of methanol was stirred with palladium on activated carbon (0.45 g) under an atmosphere of hydrogen (1 atm) for 50 min. The reaction mixture was then filtered through a pad of Celite and the filter cake was washed with methanol and methylene chloride. The filtrates were combined and concentrated, and the residue was chased with methylene chloride to give N-[valinyl]aspartic acid, α-methyl, β-tert-butyl diester a white solid (2.75 g, 99%). TLC (CH₂Cl₂/MeOH, 95/5): R_{f}=0.10.

To a turbid mixture of the above product (2.75 g, 9.11 mmol) and 1,3-dimethylindole-2-carboxylic acid (1.95 g, 10.3 mmol) in DMF (30 ml) was added 4-dimethylaminopyridine (DMAP) (1.26 g, 10.3 mmol) and 1-(3′-dimethylaminopropyl)-3-ethylcarbodiimide hydrochloride (EDAC) (2.37 g, 12.4 mmol). The reaction mixture was stirred under a nitrogen atmosphere at 0° C. for 1 hour and at room temperature for 3 hours. The reaction mixture was then partitioned between ethyl acetate and 5% KHSO₄ solution and the aqueous solution was back-extracted with ethyl acetate. The combined extracts were washed with saturated NaHCO₃ solution, water, and brine, dried over sodium sulfate, and concentrated to give a solid. The solid was triturated with ether to give N-[(1,3-dimethyl-indole-2-carbonyl)valinyl]aspartic acid, α-methyl, β-tert-butyl diester as a white powder (2.87 g, 67%). TLC (CH₂Cl₂/MeOH, 95/5): R_{f}=0.59.

An aqueous solution of lithium hydroxide (1.0 M, 2.98 ml) was added dropwise to a suspension the above product (1.41 g, 2.98 mmol) in 1,4-dioxane (10 ml). After stirring at room temperature for 30 min, the resulting clear was acidified with 1 N hydrochloric acid solution and diluted with water. The resulting white precipitate was collected by suction filtration and washed successively with water and with a small amount of ether, affording N-[(1,3-dimethyl-indole-2-carbonyl)-valinyl]aspartic acid, β-tert-butyl ester as a white powder (1.18 g, 86%). TLC(CH₂Cl₂/MeOH, 90/10): R_{f}=0.21.

To a solution of the above product (1.03 g, 2.24 mmol) and 4-methylmorphorline (0.35 ml, 3.14 mmol) in THF (20 mL) at −10C under nitrogen was added dropwise isobutyl chloroformate (0.380 ml, 2.92 mmol). The reaction mixture was stirred under nitrogen at −10° C. for 15 min and filtered. The filter cake was washed with dry THF and the filtrates were combined and cooled to 0° C. The filtrates were then treated with a freshly prepared ether solution of diazomethane (excess). After the mixture was stirred at 0° C. for 1 hour, a mixture of hydrobromic acid (48% wt. aq. solution) and acetic acid (6 ml, 1/1) was added dropwise till the gas evolution ceased. After another 5 min, the reaction mixture was concentrated and partitioned between ethyl acetate and water. The aqueous layer was back-extracted with ethyl acetate. The organic layers were combined, washed with water, saturated NaHCO₃ solution, and brine, dried over sodium sulfate, and concentrated. The residue was triturated with ether to give the title compound as a white powder (1.00 g, 83%). TLC(CH₂Cl₂/MeOH,95/5): R_{f}=0.88.

### EXAMPLE 69

### N-[(1,3-dimethyl-indole-2-carbonyl)-valinyl]-3-amino-5-(2,6-dichlorobenzoyl)oxy-4-oxo-pentanoic Acid, T-Butyl Ester

To a mixture of 2,6-dichlorobenzoic acid (0.023 g, 0.12 mmol) and potassium fluoride (0.015 g, 0.25 mmol) at room temperature under nitrogen was added N-[(1,3-dimethyl-indole-2-carbonyl)valinyl]-3-amino-5-bromo-4-oxo-pentanoic acid, tert-butyl ester (0.054 g, 0.10 mmol) in one portion. After stirring at room temperature for further 16 hrs, the mixture was partitioned between ethyl acetate and water. The organic layer was washed with water, saturated NaHCO₃ solution, and brine, dried over sodium sulfate, and concentrated. Trituration with ether gave the title compound as a white powder (0.051 g, 79%). TLC(CH₂Cl₂/MeOH, 95/5): R_{f}=0.88.

### EXAMPLE 70

### N-[N-(1,3-dimethyl-indole-2-carbonyl)-valinyl]-3-amino-5-(2,6-dichlorobenzoyl)oxy-4-oxo-pentanoic Acid

Trifluoroacetic acid (2 mL) was added to a stirred solution of N-(1,3-dimethyl-indole-2-carbonyl)-valinyl-3-amino-5-(2,6-dichlorobenzoyl)oxy-4-oxo-pentanoic acid, t-butyl ester (0.0340 g, 0.0526 mmol) in methylene chloride containing anisole (0.2 mL). The reaction mixture was stirred at room temperature under nitrogen for half an hour and concentrated. The residue was azeotroped with methylene chloride and triturated with ether to give the title compound as a white powder (0.0270 g, 87%). TLC(CH₂Cl₂/MeOH/AcOH, 20/1/1): R_{f}=0.43. MS for C₂₈H₂₉Cl₂N₃O₇, [MH]⁺590/592, [MH]⁻588/590.

Following the methods set down in Examples 69-70, the following compounds were prepared:

### EXAMPLE 71

### N-(1,3-dimethyl-indole-2-carbonyl)-valinyl-3-amino-5-(diphenylphosphinyl)oxy-4-oxo-pentanoic Acid

24% yield; TLC(CH₂Cl₂/MeOH/AcOH, 20/1/1): R_{f}=0.31. MS for C₃₃H₃₆PN₃O₇, [MH]⁺618, [MH]⁻616.

### EXAMPLE 72

### N-(1,3-dimethyl-indole-2-carbonyl)-valinyl-3-amino-5-(1-phenyl-3-(trifluoromethyl)pyrazol-5-yl)oxy-4-oxo-pentanoic Acid

49% yield; TLC(CH₂Cl₂/MeOH, 90/10): R_{f}=0.29. MS for C₃₁H₃₂F₃N₅O₆, [MH]⁺628, [MH]⁻626.

### EXAMPLE 73

### N-(1,3-dimethyl-indole-2-carbonyl)-valinyl-3-amino-5-(3-(N-phenyl)aminocarbonyl-2-naphthyl)oxy-4-oxo-pentanoic Acid

68% yield; TLC(CH₂Cl₂/MeOH, 80/20): R_{f}=0.46. MS for C₃₈H₃₈N₄O₇, [MH]⁺663, [MH]⁻661.

### EXAMPLE 74

### N-(1,3-dimethyl-indole-2-carbonyl)-valinyl-3-amino-5-(2-aminocarbonyl-1-phenyl)oxy-4-oxo-pentanoic Acid

61% yield; TLC(CH₂Cl₂/MeOH/HOAc, 8/1/1): R_{f}=0.32. MS for C₂₈H₃₂N₄O₇, [MH]⁺537, [MH]⁻535.

### EXAMPLE 75

### N-(1,3-dimethyl-indole-2-carbonyl)-valinyl-3-amino-5-(dimethylphosphinyl)oxy-4-oxo-pentanoic Acid

76% yield; TLC(CH₂Cl₂/MeOH, 90/10): R_{f}=0.12. MS for C₂₃H₃₂PN₃O₇, [MH]⁺494, [MH]⁻492.

### EXAMPLE 76

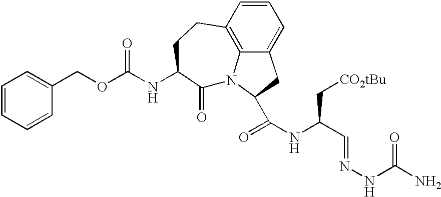

### (2S-cis)-[5-benzyloxycarbonylamino-1,2,4,5,6,7-hexahydro-4-oxoazepino [3,2,1-hi]indole-2-carbonyl)amino]-4-oxo-butanoic Acid Tert-Butyl Ester Semicarbazone

1. Preparation of (2S-cis)-5-Benzyloxycarbonylamino-1,2,4,5,6,7-Hexahydro-4-Oxoazepino[3,2,1-hi]indole-2-Carboxylic Acid, Ethyl Ester

To a solution of (2S-cis)-5-amino-1,2,4,5,6,7-hexahydro-4-oxoazepino[3,2,1-hi]indole-2-carboxylic acid, ethyl ester (0.437 g, 1.73 mmol, prepared as described in Tetrahedron Letters 36, pp. 1593-1596 (1995) and U.S. Pat. No. 5,504,080 (Apr. 2, 1996) in methylene chloride (4 mL) stirring at 0° C. was added benzyl chloroformate (0.370 mL, 2.6 mmol) and triethylamine (0.724 mL, 5.2 mmol) and the resulting mixture was stirred under nitrogen for 45 minutes. The reaction was quenched with water then partitioned between ethyl acetate and 5% aqueous potassium bisulfate solution. The aqueous layer was back-extracted two times with ethyl acetate, then the combined organic layers were washed with saturated sodium chloride solution, dried over sodium sulfate and evaporated to dryness. Purification of the crude product by flash chromatography on silica gel (S/P brand silica gel 60 Å, 230-400 mesh ASTM) eluting with ethyl acetate-hexane (2:1) gave 0.558 g (68%) of crude product. Trituration with ethyl acetate-hexane (1:4) gave 0.480 g of the title compound as white solid; m.p.: 139-140° C. TLC (ethyl acetate-hexane, 2:1): R_{f}=0.6; ¹H-NMR (300 MHz, CDCl₃): δ 7.35-7.30 (m, 5H), 7.02-6.94 (m, 3H), 6.17 (d, J=5.4 Hz, 1H), 4.15 (q, J=7.1 Hz, 2H), 3.46 (dd, J=11.0, 16.7 Hz, 1H), 3.29 (m, 1H), 3.10 (d, J=116.5, 2H), 2.35 (m, 1H), 2.16 (m, 1H), 1.23 (t, J=7.2 Hz, 3H).

2. Preparation of (2S-cis)-5-Benzyloxycarbonylamino-1,2,4,5,6,7-Hexahydro-4-Oxoazepino[3,2,1-hi]indole-2-Carboxylic Acid

To a solution of (2S-cis)-5-benzyloxycarbonylamino-1,2,4,5,6,7-hexahydro-4-oxoazepino[3,2,1-hi]indole-2-carboxylic acid, ethyl ester, (0.428 g, 1.05 mmol) in 1,4-dioxane (7.5 mL) and water (2.5 mL) was added IM aqueous lithium hydroxide (1.6 mL, 1.6 mmol) and the resulting mixture was stirred at room temperature under nitrogen for 30 minutes. The reaction mixture was acidified to pH 3 with a 5% aqueous potassium bisulfate sodium chloride solution. The aqueous layer was back-extracted two times with ethyl acetate, and the combined organic layers were dried over sodium sulfate and evaporated to dryness to yield 0.395 g (99%) of title compound as a fine white solid; m.p.: 188-189° C. TLC (methylene chloride-methanol-acetic acid, 9:1:1): R_{f}=0.55; ¹H-NMR (300 MHz, CDCl₃) δ 7.34-7.26 (m, 5H), 7.07-6.97 (m, 3H), 6.08 (d, J=5.7 Hz, 1H), 5.25 (dd, J=3.2, 9.8 Hz, 1H), 5.10 (s, 2H), 4.30 (m, 1H), 3.36 (m, 1H), 3.26 (m, 2H), 3.06 (d, J=12.0 Hz, 1H), 2.36 (m, 1H), 2.09 (m, 1H).

3. Preparation of N-Benzyloxycarbonyl-L-(N′-Methyl-N′-Methoxy)aspartamide β-(tert-Butyl Ester)

To a solution of N-(benzyloxycarbonyl)-L-aspartic acid-β-(tert-butyl)ester (14.65 g, 45.3 mmol, Bachem) in CH₂Cl₂ (150 mL) at 0° C. (ice bath) under a nitrogen atmosphere was added 1-hydroxybenzotriazole hydrate (7.29 g, 47.6 mmol, Aldrich) followed by 1-ethyl-3-(3′,3′-dimethyl-1′-aminopropyl)carbodiimide hydrochloride (9.55 g, 49.8 mmol, Sigma). After stirring at 0° C. for 15 min., N,O-dimethylhydroxylamine hydrochloride (5.10 g, 52.3 mmol, Aldrich) and N-methylmorpholine (5.8 mL, 53 mmol, Aldrich) were added. The mixture was allowed to warm to room temperature over 3 hours then stirred at room temperature for 16 hours. The solution was concentrated under vacuum and the residue partitioned between ethyl acetate-5% KHSO₄ (200 mL each). The organic phase was washed in turn with 5% KHSO₄, saturated sodium bicarbonate and saturated sodium chloride solutions; dried over anhydrous sodium sulfate and evaporated to an oil. The oil was crystallized from hexane to give the title product (16.10 g, 97% yield) as a fluffy white crystalline solid. TLC (ethyl acetate), single spot (UV and PMA): R_{f}=0.37.

A similar procedure to the one above, starting with 29.3 g of N-(benzyloxycarbonyl)-L-aspartic acid-β-(tert-butyl)ester (2-fold scale up) gave 31.18 g (94% yield) of the title product.

4. Preparation of N-(Benzyloxycarbonyl)-L-Aspartic Acid Semicarbazone β-(tert-Butyl)Ester

To a solution of N-(benzyloxycarbonyl)-L—(N′-methyl-N′-methoxy)aspartamide β-(tert-butyl ester) (15.50 g, 42.3 mmol) in anhydrous ether (400 mL) at 0° C. (ice bath) under a nitrogen atmosphere was added dropwise to a 1.0 M solution of LiAlH₄ in ether (22.0 mL, 22.0 mmol, Aldrich) at such a rate as to keep the reaction solution temperature between 0-5° C. (addition time 15-20 min). After the addition of the lithium aluminum hydride reagent was complete, the mixture was stirred at 0-5° C. for 1 hr, then quenched by the dropwise addition of 0.3 N KHSO₄ solution (100 mL). The resultant mixture was transferred to a separatory funnel adding sufficient 5% KHSO₄ solution (75 mL) to dissolve the solids. The organic phase was separated and the combined aqueous washes back-extracted with ether (100 mL). The combined ether extracts were washed with saturated NaCl solution, dried over anhydrous sodium sulfate and concentrated in vacuo with minimal heating. TLC (ethyl acetate): streaky spot (UV and PMA) R_{f}=0.48. TLC (methanol/methylene chloride, 1:9) major spot (UV and PMA): R_{f}=0.75.

The crude aldehyde was immediately taken up in aqueous ethanol (45 ML water/I 05 mL alcohol), placed in an ice bath and treated with sodium acetate (3.82 g, 46.6 mmol) and semicarbazide hydrochloride (5.20 g, 46.6 mmol, Aldrich). The mixture was stirred at 0° C. (ice bath) under a nitrogen atmosphere for 3 hrs, allowed to warm to room temperature, and stirred overnight (16 hrs). Most of the ethanol was removed under vacuum and the residue partitioned between ethyl acetate and water (100 mL each). The organic phase was washed sequentially with 5% KHSO₄, saturated sodium bicarbonate and saturated sodium chloride solutions; dried over anhydrous sodium sulfate and evaporated to dryness. The crude product of this reaction was combined with that of two similar procedures starting with 15.40 g and 4.625 g of N-(benzyloxycarbonyl)-L-(N′-methyl-N′-methoxy)aspartamide b-(tert-butyl ester) (total: 35.525 g, 97 mmol) and these combined products were purified by flash chromatography on silica gel eluting with acetone/methylene chloride (3:7) then methanol-acetone-methylene chloride (0.5:3:7) to give pure title product (27.73 g, 78.5%) as a colorless foam. TLC (MeOH—CH₂Cl₂, 1:9): single spot (UV and PMA), R_{f}=0.51.

5. Preparation of L-Aspartic Acid Semicarbazone β-(tert-Butyl) Ester, p-Toluenesulfonate Salt

To a solution of N-(benzyloxycarbonyl)-L-aspartic acid semicarbazone β-(tert-butyl)ester (13.84 g, 38.0 mmol) in absolute ethanol (250 mL) was added 10% Pd/C (1.50 g, Aldrich) and the resulting mixture stirred under an atmosphere of hydrogen (balloon) until TLC (methanol/methylene chloride, 1:9) indicated complete consumption of the starting material (60 min). Note: It is important to follow this reaction closely since the product can be over-reduced. The mixture was filtered though Celite and evaporated to an oil. The oil was chased with methylene chloride (2×75 mL) then with methylene chloride/toluene (1:1, 75 mL) to give the crude amine as a white crystalline solid. TLC (EtOAc-pyridine-AcOH—H₂O; 60:20:5:10) single spot (UV and PMA) R_{f}=0.24. Note: In this TLC system, any over-reduced product will show up immediately below the desired product, R_{f}=0.18 (PMA only).

The crude amine was taken up in CH₃CN (60 mL) and treated with a solution of p-toluenesulfonic acid monohydrate (7.22 g, 38.0 mmol) in acetonitrile (60 mL). The crystalline precipitate was collected, washed with acetonitrile and ether, and air-dried to give the title compound (13.95 g, 92% yield) as a white, crystalline solid.

The optical purity of this material was checked by conversion to the corresponding Mosher amide [1.05 equiv (R)-(−)-α-methoxy-α-(trifluoromethyl)phenylacetyl chloride, 2.1 equivalents of i-Pr₂NEt in CH₂Cl₂, room temperature, 30 min]. The desired product has a doublet at 7.13 ppm (1H, d, J=2.4 Hz, CH═N) while the corresponding signal for its diastereomer is at 7.07 ppm. The optical purity of the title compound obtained from the above procedure is typically >95:5.

6. (2S-cis)-[5-Benzyloxycarbonylamino-1,2,4,5,6,7-Hexahydro-4-Oxoazepino[3,2,1-hi]indole-2-Carbonyl)amino]-4-Oxo-butanoic Acid tert-Butyl Ester Semicarbazone

To a solution of (2S-cis)-5-benzyloxycarbonylamino-1,2,4,5,6,7-hexahydro-4-oxoazepino[3,2,1-hi]indole-2-carboxylic acid (0.375 g, 0.989 mmol) in methylene chloride (7 mL) stirring at 0° C. under nitrogen was added 1-hydroxybenzotriazole hydrate (0.182 g, 1.19 mmol) and 1-ethyl-3-(3′,3′-dimethyl-1′-aminopropyl)carbodiimide hydrochloride (0.284 g, 1.48 mmol). After 15 minutes L-aspartic acid semicarbazone b-(tert-butyl) ester, p-toluenesulfonate salt(0.386 g, 0.989 mmol) and N-methylmorpholine (0.163 mL, 1.48 mmol) were added and the resultant reaction mixture allowed to come to room temperature within 1 hour. After stirring overnight, the reaction mixture was diluted with ethyl acetate and washed successively with 5% potassium bisulfate and saturated sodium chloride solutions; dried over sodium sulfate and evaporated to dryness. Purification of the crude product by flash chromatography on silica gel (S/P brand silica gel 60 Å, 230-400 mesh ASTM) eluting with 2% methanol-methylene chloride gave 0.463 g (79%) of the title compound as a white foam. TLC (methylene chloride-methanol, 9:1): R_{f}=0.5. ¹H-NMR (300 MHz, CDCl₃): δ 8.42 (s, 1H), 7.82 (d, J=8.1 Hz, 1H), 7.32 (m, 5H), 7.07 (m, 3H), 5.94 (d, J=6.3 Hz, 1H), 5.26 (d, J=9 Hz, 1H), 5.10 (s, 2H), 4.82 (m, 1H), 4.35 (m, 1H), 3.56 (d, J=18 Hz, 1H), 3.27 (m, 2H), 3.07 (m, 1H), 2.64 (dd, J=4.7, 15.8 Hz, 1H), 2.44 (dd, J=6.6, 15.9 Hz, 2H), 2.22 (m, 1H), 130 (s, 9H). Mass spectrum: m/z 593 (M+H).

### EXAMPLE 77

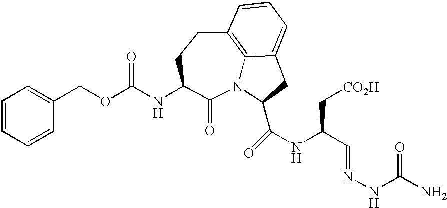

### (2-cis)-[5-benzyloxycarbonylamino-1,2,4,5,6,7-hexadydro-4-oxoazepino [3,2,1-hi]indole-2-carbonyl)-amino]-4-oxo-butanoic Acid Semicarbazone

To a solution of (2S-cis)-[5-benzyloxycarbonylamino-1,2,4,5,6,7-bexahydro-4-oxoazepino [3,2,1-hi]indole-2-carbonyl)amino]-4-oxo-butanoic acid tert-butyl ester semicarbazone (0.214 g, 0.362 mmol) in methylene chloride (1.5 mL) was added anisole (0.5 mL, 4.34 mmol) followed by trifluoroacetic acid (0.75 mL). After stirring at room temperature under nitrogen for 2 hours the reaction mixture was diluted with methylene chloride and evaporated, then chased twice with methylene chloride to give the title compound (0.195 g). TLC (methylene chloride-methanol, 95:5), R_{f}=0.2. ¹H-NMR (300 MHz, CDCl₃) δ 9.77 (bs, 1H), 8.32 (d, J=12 Hz, 1H), 8.12 (d, J-7.8 Hz, 1H), 7.31-7.27 (m, 5H) 7.13-7.04 (m, 3H), 6.64 (m, 1H) 5.32 (d, J=9.9 Hz, 1H), 5.12 (s, 2H), 4.86 (m, 1H), 4.41 (m, 1H), 3.56 (d, J=15 Hz, 1H), 3.25 (m, 2H), 3.10 (m, 2H), 2.64 (m, 2H), 2.28 (m, 2H).

### EXAMPLE 78

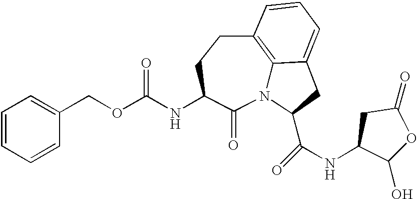

### (2S-cis)-5-[benzyloxycarbonylamino-1,2,4,5,6,7-hexahydro-4-oxoazepino [3,2,1-hi]indole-2-carbonyl)-amino]-4-oxo-butanoic Acid

(2-cis)-[5-Benzyloxycarbonylamino-1,2,4,5,6,7-hexadydro-4-oxoazepino[3,2,1-hi]indole-2-carbonyl)-amino]-4-oxo-butanoic acid semicarbazone (0.195 g, 0.36 mmol) was treated with a 3:1:1 solution of methanol-acetic acid-37% formaldehyde (2 mL) and the resulting mixture stirred under nitrogen for 1.5 hours. The reaction mixture was diluted with water, methanol removed by evaporation, then the remaining mixture lyophilized. Purification of the crude product by flash chromatography on reverse phase gel (MCI gel, CHP-20P, 75-150 micron)eluting with a 10%-80% methanol-water gradient gave 0.073 g, (42%) of the title compound as a white solid after lyophilization; m.p. 101-104° C. TLC (methylene chloride-methanol-acetic acid, 97:2.5:0.5) R_{f}=0.45. ¹H-NMR (300 MHz, CDCl₃) δ 7.45 (m, 1H), 7.30 (s, 5H), 7.07 (d, J=3.3 Hz, 1H), 7.00 (d, J=4.8 Hz, 2H), 6.12 (m, 1H), 5.17 (d, J=9.6 Hz, 1H), 5.07 (s, 2H), 4.49 (m, 1H), 4.28 (m, 1H), 3.46 (d, J=9.9 Hz, 1H), 3.30-3.12 (m, 2H), 3.04-2.99 (m, 1H), 2.83-2.76 (m, 1H), 2.46-2.33 (m, 2H), 2.03 (bs, 1H). Mass spectrum: m/z 480 (M+H).

### EXAMPLE 79

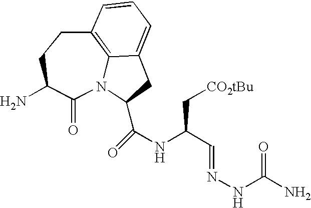

### (2S-cis)-[5-amino-1,2,3,4,5,6,7-hexahydro-4-oxoazepino [3,2,1-hi]indole-2-carbonyl)-amino]-4-oxo-butanoic Acid Tert-Butyl Ester Semicarbazone

10% Palladium on carbon (0.180 g) was added to a solution of (2S-cis)-[5-benzyloxycarbonylamino-1,2,3,4,5,6,7-hexahydro-4-oxoazepino[3,2,1-hi]indole-2-carbonyl)-amino]-4-oxo-butanoic acid tert-butyl ester semicarbazone (0.308 g, 0.520 mmol) in methanol (27 mL) and the resulting mixture was hydrogenated using a balloon of hydrogen (1 atm, R.T.) for 18 hours. The mixture was filtered through Celite, evaporated to dryness, then chased two times with toluene to give the title compound as an off-white solid (0.215 g). TLC (methylene chloride-methanol, 9:1) R_{f}=0.15. ¹H-NMR (300 MHz, CDCl₃) δ 8.53 (s, 1H), 7.89 (d, J=7.6 Hz, 1H), 7.13 (m, 3H), 5.21 (dd, J=2.3, 10.14 Hz, 1H), 4.82 (m, 1H), 3.52 (m, 1H), 3.24 (dd, J=10.3, 16.3 Hz, 1H), 3.03 (m, 2H), 2.62 and 2.42 (AB, dd, J=4.2, 7.1, 15.7 Hz, 2H), 2.19 (m, 1H), 1.32 (s, 9H).

### EXAMPLE 80

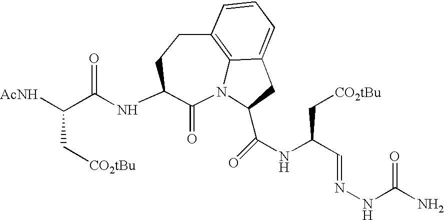

### (2S-cis)-[5-(N-acetyl-(S)-aspartyl-B-tert-butyl Ester)Amino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of N-acetyl aspartic acid, β-tert-butyl ester (0.120 g, 0.517 mmol) in methylene chloride (1.5 mL) stirring at 0° C. under nitrogen was added 1-hydroxybenzotriazole hydrate (0.086 g, 0.564 mmol) and 1-(3-dimethylaminopropyl)-3-ethylcarbodiimide hydrochloride (0.135 g, 0.705 mmol). After 15 minutes, a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.213 g, 0.47 mmol) in methylene chloride (2 mL) was added and the reaction was allowed to come to room temperature over 1 hour. After stirring overnight, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60 Å, 230-400 mesh ASTM) eluting with 5% then 10% methanol-methylene chloride gave 0.126 g (41%) of the title compound as a white solid. TLC (methylene chloride-methanol, 9:1) R^{f}=0.4. ¹H-NMR (300 MHz, CDCl₃) δ 9.63 (s, 1H), 8.32 (d, J=7.8 Hz, 1H), 7.82 (d, J=6.6 Hz, 1H), 7.53 (d, J=4.8 Hz, 1H), 7.09 (m, 1H), 7.00 (m, 2H), 5.18 (d, J=8.1 Hz, 1H), 4.86 (m, 1H), 4.39 (m, 1H), 3.01 (m, 1H), 2.92 (dd, J=4.2, 14.7 Hz, 1H) 2.68 (d, J=12.3 Hz, 1H), 2.52 (m, 2H), 2.51 (m, 2H), 2.03 (s, 3H), 1.39 (s, 9H), 1.24 (s, 9H).

### EXAMPLE 81

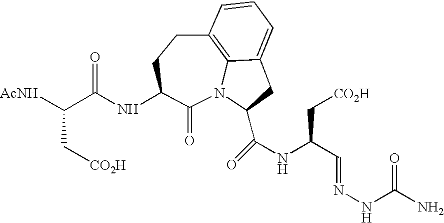

### (2 S-cis)-[5-(N-acetyl-(S)-aspartyl)Amino-1,2,3,4,5,6,7-hexahydro-4-oxoazepino [3,2,1-hi]indole-2-carbonyl)-amino]-4-oxo-butanoic Acid Semicarbazone

To a solution of (2S-cis)-[5-(N-acetyl-(S)-aspartyl-b-tert-butyl ester)amino-1,2,3,4,5,6,7-hexahydro-4-oxoazepino[3,2,1-hi]indole-2-carbonyl)-amino]-4-oxo-butanoic acid tert-butyl ester semicarbazone (0.117 g, 0.178 mmol) in methylene chloride (1 mL) was added anisole (0.5 mL) followed by trifluoroacetic acid (1 mL). After stirring at room temperature under nitrogen for 2 hours, the reaction mixture was diluted with methylene chloride and evaporated, then chased twice with methylene chloride to give the title compound (0.099 g). TLC (methylene chloride-methanol-acetic acid, 13:6:1) R_{f}=0.2. Mass spectrum: m/z 560 (M+H).

### EXAMPLE 82

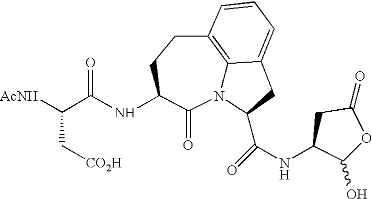

### (2S-cis)-[5-(N-acetyl-(S)-aspartyl)amino-1,2,3,4,5,6,7-hexahydro-4-oxoazepino[3,2,1-hi]indole-2-carbonyl)-amino]-4-oxo-butanoic Acid

(2S-cis)-[5-(N-Acetyl-(S)-aspartyl)amino-1,2,3,4,5,6,7-hexahydro-4-oxoazepino[3,2,1-hi]indole-2-carbonyl)-amino]-4-oxo-butanoic acid semicarbazone (0.097 g, 0.177 mmol), was treated with a 3:1:1 solution of methanol-acetic acid-37% formaldehyde (2 mL) and the resulting mixture stirred under nitrogen for 1.5 hours. The reaction mixture was then diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.050 g (56%) of the title compound as a white solid after lyophilization; m.p. 160-175° C. (dec). TLC (methylene chloride-methanol-acetic acid, 13:6:1) R_{f}=0.3. Mass spectrum: m/z 503 (M+H).

### EXAMPLE 83

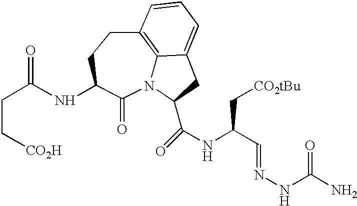

### (2S-cis)-[5-succinylamino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.197 g, 0.435 mmol) in methylene chloride (6 mL) stirring at 0° C. under nitrogen was added succinic anhydride (0.057 g, 0.566 mmol), followed by pyridine (0.052 mL, 0.653 mmol). After stirring at room temperature under nitrogen for 3 hours, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60 Å, 230-400 mesh ASTM) eluting with 10% methanol-methylene chloride then 80:19:1 methylene chloride-methanol-acetic acid gave 0.216 g (88%) of the title compound as a white solid. TLC (methylene chloride-methanol-acetic acid, 8:1:1) R_{f}=0.5. Mass spectrum: m/z 557 (M−H).

### EXAMPLE 84

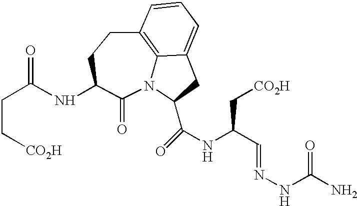

### (2S-cis)-[5-succinylamino-1,2,3,4,5,6,7-hexahydro-4-oxoazepino[3,2,1-hi]indole-2-carbonyl)-amino]-4-oxo-butanoic Acid Semicarbazone

To a solution of (2S-cis)-[5-succinylamino-1,2,3,4,5,6,7-hexahydro-4-oxoazepino[3,2,1-hi]indole-2-carbonyl)-amino]-4-oxo-butanoic acid tert-butyl ester semicarbazone (0.191 g, 0.342 mmol) in methylene chloride (1 ML) was added anisole (0.5 mL) followed by trifluoroacetic acid (1 mL). After stirring at room temperature under nitrogen for 2 hours, the reaction mixture was diluted with methylene chloride and evaporated, then chased twice with methylene chloride to give the title compound (0.210 g). TLC (methylene chloride-methanol-acetic acid, 8:1:1) R^{f}=0.4. Mass spectrum: m/z 503 (M+H).

### EXAMPLE 85

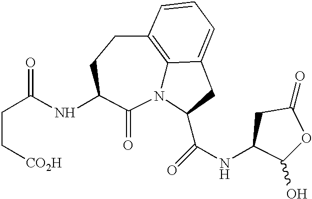

### (2S-cis)-[5-succinylamino-1,2,3,4,5,6,7-hexahydro-4-oxoazepino[3,2,1-hi]indole-2-carbonyl)-amino]-4-oxo-butanoic Acid

(2S-cis)-[5-Succinylamino-1,2,3,4,5,6,7-hexahydro-4-oxoazepino[3,2,1-hi]indole-2-carbonyl)-amino]-4-oxo-butanoic acid semicarbazone (0.208 g, ca. 0.342 mmol), was treated with a 3:1:1 solution of methanol-acetic acid-37% formaldehyde (3 mL), and the resulting mixture stirred under nitrogen for 1.5 hours. The reaction mixture was then diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.064 g (42%) of the title compound as a white solid after lyophilization; m.p. 145-160° C. (dec). TLC (methylene chloride-methanol-acetic acid, 8:1:1) R_{f}=0.45. Mass spectrum: m/z 446 (M+H).

### EXAMPLE 86

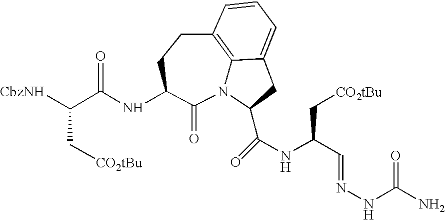

### (2S-cis)-[5-(N-benzyloxycarbonyl-(S)-aspartyl-B-tert-butyl Ester)Amino-1,2,3,4,5,6,7-hexahydro-4-oxoazepino[3,2,1-hi]indole-2-Carbonyl)-amino]-4-oxo-butanoic Acid Tert-Butyl Ester Semicarbazone

To a solution of N-benzyloxycarbonyl-(S)-aspartyl-p-tert-butyl ester (0.169 g, 0.521 mmol) in methylene chloride (1.5 mL) stirring at 0° C. under nitrogen was added 1-hydroxybenzotriazole hydrate (0.087 g, 0.569 mmol) and 1-(3-dimethylaminopropyl)-3-ethylcarbodiimide hydrochloride (0.136 g, 0.711 mmol). After 15 minutes, a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.217 g, 0.474 mmol) in methylene chloride (2 mL) was added and the reaction was allowed to come to room temperature within 1 hour. After stirring overnight, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60A, 230-400 mesh ASTM) eluting with 2% then 5% methanol-methylene chloride gave 0.244 g (67%) of the title compound as an off-white solid. TLC (methylene chloride-methanol, 9:1) R_{f}=0.55. ¹H-NMR (300 MHz, CDCl₃) δ 9.13 (s, 1H), 7.85 (d, J=6 Hz, 1H), 7.56 (d, J=5.7 Hz, 1H) 7.23 (m, 5H), 7.08 (m, 1H), 7.00 (m, 2H), 5.13 (m, 3H) 4.77 (m, 1H), 4.62 (m, 1H), 4.43 (m, 1H), 3.60 (d, J=16 Hz, 1H), 3.22 (m, 2H), 2.98 (m, 1H), 2.83 (d, J=15.3 Hz, 1H), 2.65 and 2.36 (AB, dd, J=4.2, 7.7, 16.9 Hz, 2H), 2.42 (m, 1H), 2.10 (m, 1H), 1.35 (s, 9H), 1.24 (s, 9H).

### EXAMPLE 87

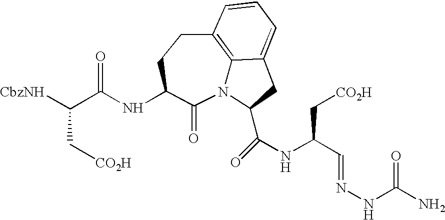

### (2S-cis)-[5-(N-benzyloxycarbonyl-(S)-aspartyl)amino-1,2,3,4,5,6,7-hexahydro-4-oxoazepino[3,2,1-hi]indole-2-carbonyl)-amino]-4-oxo-butanoic Acid Semicarbazone

To a solution of (2S-cis)-[5-(N-Benzyloxycarbonyl-(S)-aspartyl-b-tert-butyl ester)amino-1,2,3,4,5,6,7-hexahydro-4-oxoazepino[3,2,1-hi]indole-2-carbonyl)-amino]-4-oxo-butanoic acid tert-butyl ester semicarbazone (0.217 g, 0.289 mmol) in methylene chloride (1 mL) was added anisole (0.5 mL) followed by trifluoroacetic acid (1 mL). After stirring at room temperature under nitrogen for 3 hours, the reaction mixture was diluted with methylene chloride and evaporated, then chased twice with methylene chloride to give the title compound (0.193 g). TLC (methylene chloride-methanol, 9:1) R_{f}=0.35. Mass spectrum: m/z 652 (M+H).

### EXAMPLE 88

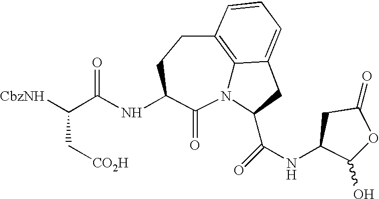

### (2S-cis)-[5-(N-benzyloxycarbonyl-(S)-aspartyl)amino-1,2,3,4,5,6,7-hexahydro-4-oxoazepino[3,2,1-hi]indole-2-carbonyl)-amino]-4-oxo-butanoic Acid

(2S-cis)-[5-(N-Benzyloxycarbonyl-(S)-aspartyl)amino-1,2,3,4,5,6,7-hexahydro-4-oxoazepino[3,2,1-hi]indole-2-carbonyl)-amino]-4-oxo-butanoic acid semicarbazone (0.191 g, 0.29 mmol), was treated with a 3:1:1 solution of methanol-acetic acid-37% formaldehyde (2 mL) and the resulting mixture stirred under nitrogen for 2 hours. The reaction mixture was then diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.111 g. (64%) of the title compound as a white solid after lyophilization; m.p. 140-144° C. (dec.). TLC (methylene chloride-methanol, 9:1) R_{f}=0.4. Mass spectrum: m/z 593 (M−H).

### EXAMPLE 89

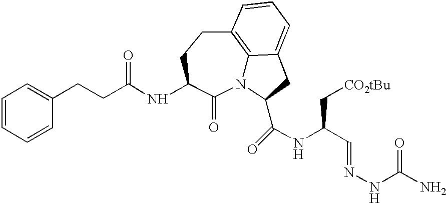

### (2S-cis)-[5-dihydrocinnamylamino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of dihydrocinnamic acid (0.169 g, 0.521 mmol) in methylene chloride (1.5 mL) stirring at 0° C. under nitrogen was added 1-hydroxybenzotriazole hydrate (0.088 g, 0.576 mmol) and 1-(3-dimethylaminopropyl)-3-ethylcarbodiimide hydrochloride (0.127 g, 0.665 mmol). After 15 minutes, a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.203 g, 0.443 mmol) in methylene chloride (2 mL), was added and the reaction was allowed to come to room temperature within 1 hour. After stirring overnight, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60 Å, 230-400 mesh ASTM) eluting with 2 then 5% methanol-methylene chloride gave 0.208 g (79%) of the title compound as an off-white solid. TLC (methylene chloride-methanol, 9:1) R_{f}=0.7. ¹H-NMR (300 MHz, CDCl₃) δ 8.82 (s, 1H), 7.72 (d, J=8.1 Hz, 1H), 7.19 (m, 5H), 7.06 (m, 1H), 7.01 (m, 2H), 6.76 (d, J=6.3, 1H), 5.23 (d, J=8.4 Hz, 1H), 4.84 (m, 1H), 4.50 (m, 1H), 3.48 (m, 1H), 3.26 (m, 2H), 3.05 (m, 1H), 2.94 (m, 2H), 2.53 (m, 4H), 2.28 (m, 1H), 2.06 (m, 1H), 1.29 (s, 9H). Mass spectrum: m/z 591 (M+H).

### EXAMPLE 90

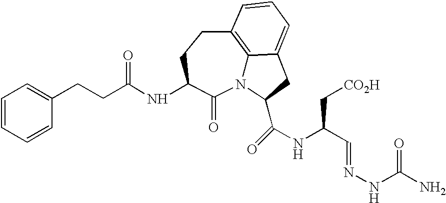

### (2S-cis)-[5-dihydrocinnamylamino-1,2,3,4,5,6,7-hexahydro-4-oxoazepino[3,2,1-hi]indole-2-carbonyl)-amino]-4-oxo-butanoic Acid Semicarbazone

To a solution of (2S-cis)-[5-dihydrocinnamylamino-1,2,3,4,5,6,7-hexahydro-4-oxoazepino[3,2,1-hi]indole-2-carbonyl)-amino]-4-oxo-butanoic acid tert-butyl ester semicarbazone (0.189 g, 0.320 mmol) in methylene chloride (1 mL) was added anisole (0.5 mL) followed by trifluoroacetic acid (1 mL). After stirring at room temperature under nitrogen for 3 hours, the reaction mixture was diluted with methylene chloride and evaporated, then chased twice with methylene chloride to give the title compound (0.183 g). TLC(methylene chloride-methanol, 9:1) R_{f}=0.25. Mass spectrum: m/z 535 (M+H).

### EXAMPLE 91

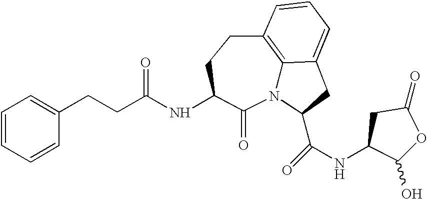

### (2S-cis)-[5-dihydrocinnamylamino-1,2,3,4,5,6,7-hexahydro-4-oxoazepino [3,2,1-hi]indole-2-carbonyl)-amino]-4-oxo-butanoic Acid

(2S-cis)-[5-Dihydrocinnamylamino-1,2,3,4,5,6,7-hexahydro-4-oxoazepino[3,2,1-hi]indole-2-carbonyl)-amino]-4-oxo-butanoic acid semicarbazone (0.181 g, ca. 0.320 mmol), was treated with a 3:1:1 solution of methanol-acetic acid-37% formaldehyde (2 mL) and the resulting mixture stirred under nitrogen for 4 hours. The reaction mixture was then diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.075 g (47%) of the title compound as a white solid after lyophilization; m.p. 78-81° C. TLC (methylene chloride-methanol, 9:1) R_{f}=0.45. ¹H-NMR (300 MHz, DMSO d6): δ 8.58 (m, 1H), 8.30 (d, J=7.5 Hz, 1H), 7.24 (m, 5H), 7.08 (m, 2H), 6.99 (m, 1H), 5.04 (d, J=9.3 Hz, 1H), 4.39 (m, 1H), 4.19 (m, 1H), 3.46 (m, 1H), 3.05 (m, 2H), 2.93 (d, J=16.8 Hz, 2H), 2.83 (m, 2H), 2.00 (d, J=5.1 Hz, 2H). Mass spectrum: m/z 478 (M+H).

### EXAMPLE 92

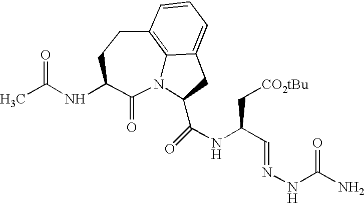

### (2S-cis)-[5-acetylamino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of (2S-cis)-[5-amino-1,2,3,4,5,6,7-hexahydro-4-oxoazepino [3,2,1-hi]indole-2-carbonyl)-amino]-4-oxo-butanoic acid tert-butyl ester semicarbazone (0.222 g, 0.490 mmol) in pyridine (3 mL) at room temperature under nitrogen was added acetic anhydride (0.07 mL, 0.735 mmol). After stirring overnight, the reaction mixture was diluted with methylene chloride and evaporated to give a foam. This was taken up in ethyl acetate and washed successively with 5% potassium bisulfate and saturated sodium chloride solutions; dried (sodium sulfate) and evaporated to dryness to give 0.130 g (53%) of the title compound as an off-white solid. TLC (methylene chloride-methanol, 9:1) R_{f}=0.55. ¹H-NMR (300 MHz, CDCl₃): δ 8.75 (s, 1H), 7.75 (d, J=8.4 Hz, 1H), 7.08 (m, 1H), 7.01 (m, 2H), 6.87 (d, J=6.3 Hz, 1H), 5.25 (d, J=8.1 Hz, 1H), 4.84 (m, 1H), 4.52 (m, 1H), 3.50 (m, 1H), 3.28 (m, 2H), 3.02 (m, 1H), 2.55 and 2.46 (AB, dd, J=4.2, 7.1, 15.7 Hz, 2H), 2.36 (m, 1H), 2.18 (m, 1H), 2.02 (s, 3H), 1.31 (s, 9H).

### EXAMPLE 93

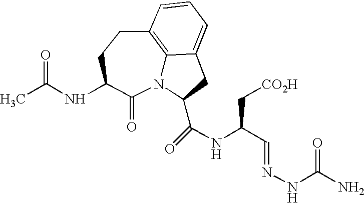

(2S-cis)-[5-acetylamino-1,2,3,4,5,6,7-hexahydro-4-oxoazepino[3,2,1-hi]indole-2-carbonyl)-amino]-4-oxo-butanoic Acid Semicarbazone

To a solution of (2S-cis)-[5-acetylamino-1,2,3,4,5,6,7-hexahydro-4-oxoazepino[3,2,1-hi]indole-2-carbonyl)-amino]-4-oxo-butanoic acid tert-butyl ester semicarbazone (0.112 g, 0.224 mmol) in methylene chloride (1 mL) was added anisole (0.5 mL) followed by trifluoroacetic acid (1 mL). After stirring at room temperature under nitrogen for 2.5 hours, the reaction mixture was diluted with methylene chloride and evaporated, then chased twice with methylene chloride to give the title compound (0.117 g). TLC (methylene chloride-methanol, 9:1) R_{f}=0.15. Mass spectrum: m/z 445 (M+H).

### EXAMPLE 94

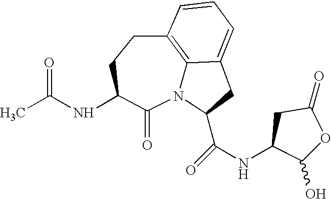

### (2S-cis)-[5-acetylamino-1,2,3,4,5,6,7-hexahydro-4-oxoazepino[3,2,1-hi]indole-2-carbonyl)-amino]-4-oxo-butanoic Acid

(2S-cis)-[5-Acetylamino-1,2,3,4,5,6,7-hexahydro-4-oxoazepino[3,2,1-hi]indole-2-carbonyl)-amino]-4-oxo-butanoic acid semicarbazone (0.115 g, ca. 0.224 mmol) was treated with a 3:1:1 solution of methanol-acetic acid-37% formaldehyde (2 mL) and the resulting mixture stirred under nitrogen for 5 hours. The reaction mixture was diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.044 g (51%) of the title compound as a white solid after lyophilization; m.p. 210-215° C. (dec). TLC (methylene chloride-methanol-acetic acid, 44:5:1) R_{f}=0.45. Mass spectrum: m/z 388 (M+H).

### EXAMPLE 95

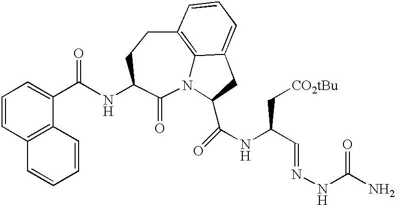

### (2S-cis)-[5-(1-naphthoyl)amino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of 1-naphthoic acid (0.072 g, 0.417 mmol) in methylene chloride (1.5 mL) stirring at 0° C. under nitrogen was added 1-hydroxybenzotriazole hydrate (0.077 g, 0.501 mmol.) and 1-(3-dimethylaminopropyl)-3-ethylcarbodiimide hydrochloride (0.120 g, 0.626 mmol). After 15 minutes, a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.189 g, 0.147 mmol) in methylene chloride (2 mL), was added and the reaction was allowed to come to room temperature within 1 hour. After stirring a total of 5 hours, the reaction mixture was diluted with ethyl acetate and washed successively with 5% potassium bisulfate and saturated sodium chloride solutions, dried (sodium sulfate) and evaporated to dryness. Purification of the crude product by flash chromatography on silica gel (S/P brand silica gel 60 Å, 230-400 mesh ASTM eluting with 5% methanol-methylene chloride gave 0.168 g (66%) of the title compound as an off-white solid; m.p. 103-105° C. (dec.). TLC (methylene chloride-methanol, 9:1) R_{f}=0.6. Mass spectrum: m/z 613 (M+H). ¹H-NMR (300 MHz, CDCl₃) δ 9.09 (bs, 1H), 8.38 (d, J=8.4 Hz, 1H), 7.82-7.93 (m, 3H), 7.70 (d, J=6.3 Hz, 1H), 7.45-7.58 (m, 3H), 7.37 (d, J=6.6 Hz, 1H), 7.06-7.15 (m, 4H), 5.30 (d, J=8.4 Hz, 1H), 4.80-4.85 (m, 2H), 3.57 (d, J=3.6 Hz, 1H), 3.30-3.45 (m, 2H), 3.16 (m, 1H), 2.59-2.65 (m, 2H), 2.27-2.49 (m, 2H), 1.29 (s, 9H).

### EXAMPLE 96

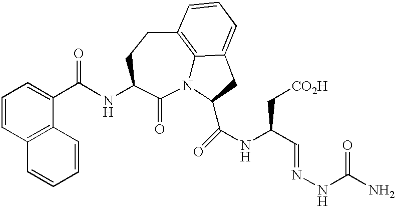

### (2S-cis)-[5-(1-naphthoyl)amino-1,2,3,4,5,6,7-hexahydro-4-oxoazepino [3,2,1-hi]indole-2-carbonyl)-amino]-4-oxo-butanoic Acid Semicarbazone

To a solution of (2S-cis)-[5-(1-naphthoyl)amino-1,2,3,4,5,6,7-hexahydro-4-oxoazepino[3,2,1-hi]indole-2-carbonyl)-amino]-4-oxo-butanoic acid tert-butyl ester semicarbazone (0.106 g, 0.173 mmol) in methylene chloride (1 mL) was added anisole (0.5 mL) followed by trifluoroacetic acid (1 mL). After stirring at room temperature under nitrogen for 3 hours, the reaction mixture was diluted with methylene chloride and evaporated, then chased twice with methylene chloride to give the title compound (0.110 g). TLC (methylene chloride-methanol, 9:1) R^{f}=0.3.

### EXAMPLE 97

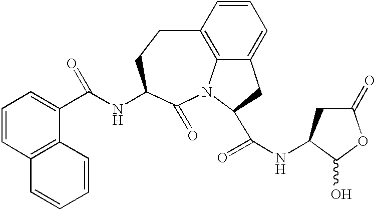

### (2S-cis)-[5-(1-naphthoyl)amino-1,2,3,4,5,6,7-hexahydro-4-oxoazepino [3,2,1-hi]indole-2-carbonyl)-amino]-4-oxo-butanoic Acid

(2S-cis)-[5-(1-Naphthoyl)amino-1,2,3,4,5,6,7-hexahydro-4-oxoazepino[3,2,1-hi]indole-2-carbonyl)-amino]-4-oxo-butanoic acid semicarbazone (0.110 g, ca. 0.173 mmol) was treated with a 3:1:1 solution of methanol-acetic acid-37% formaldehyde (3 mL) and the resulting mixture stirred under nitrogen for 5 hours. The reaction mixture was then diluted with water, methanol removed by evaporation, then the remaining mixture lyophilized. Purification of the crude product by flash chromatography on silica gel (S/P brand silica gel 60 Å, 230-400 mesh ASTM) eluting with a 5%-20% methanol-methylene chloride gradient gave 0.076 g (86%) of the title compound as a white solid; m.p. 202-203° C. (dec). TLC(methylene chloride-methanol-acetic acid, 20:1:1) R_{f}=0.3. Mass spectrum: m/z 498 (M−H). ¹H-NMR (300 MHz, CDCl₃) δ 9.38 (bs, 1H), 8.94 (m, 1H), 8.56 (m, 1H), 8.36 (m, 1H), 7.94-8.02 (m, 2H), 7.68 (d, J=6.9 Hz, 1H), 7.51-7.59 (m, 3H), 7.07-7.13 (m, 2H), 6.97 (m, 1H), 5.20 (d, J=10.5, 1Hz), 4.67 (m, 1H), 4.15 (m, 1H), 3.49 (m, 1H), 2.95-3.23 (m, 2H), 2.53 (m, 1H), 2.22-2.34 (m, 2H).

### EXAMPLE 98

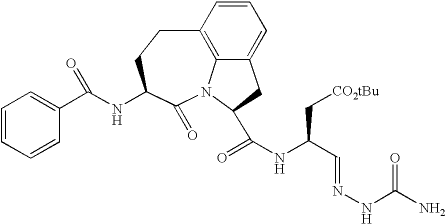

### (2S-cis)-[5-benzoylamino-1,2,3,4,5,6,7-hexahydro-4-oxoazepino[3,2,1-hi]indole-2-carbonyl)-amino]-4-oxo-butanoic Acid Tert-Butyl Ester Semicarbazone

To a solution of (2S-cis)-[5-amino-1,2,3,4,5,6,7-hexahydro-4-oxoazepino[3,2,1-hi]indole-2-carbonyl)-amino]-4-oxo-butanoic acid tert-butyl ester semicarbazone (0.121 g, 0.264 mmol) in methylene chloride (2.5 mL) stirring at 0° C. under nitrogen was added triethylamine (0.055 mL, 0.396 mmol), followed by benzoyl chloride (0.037 mL, 0.317 mmol). After stirring at room temperature under nitrogen for 1 hour, the reaction mixture was diluted with ethyl acetate and washed successively with 5% potassium bisulfate, saturated sodium bicarbonate and saturated sodium chloride solutions; dried (sodium sulfate) and evaporated to dryness. Purification of the crude product by flash chromatography on silica gel (S/P brand silica gel 60 Å, 230-400 mesh ASTM) eluting with 10% hexane-ethyl acetate, 100% ethyl acetate, then 10% methanol-ethyl acetate gave 0.073 g (49%) of the title compound as an off-white solid. TLC (methylene chloride-methanol, 9:1) R_{f}=0.7. Mass spectrum: m/z 563 (M+H). ¹H-NMR (300 MHz, CDCl₃): δ 8.61 (bs, 1H), 7.83-7.86 (m, 2H), 7.47-7.53 (m, 3H), 7.06-7.12 (m, 3H), 5.30 (dd, J-2.2, 7.8 Hz, 1H), 4.89 (m, 1H), 4.72 (m, 1H), 3.60 (dd, J=16.5 Hz, 1H), 3.36 (m, H), 3.19 (m, 1H), 2.69 (dd, J=4.4, 11.7 Hz, 1H), 2.52 (m, 1H), 2.29 (m, 1H), 1.34 (s, 9H).

### EXAMPLE 99

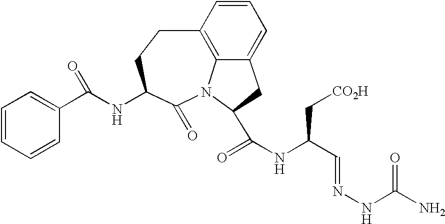

### (2S-cis)-[5-benzoylamino-1,2,3,4,5,6,7-hexahydro-4-oxoazepino[3,2,1-hi]indole-2-carbonyl)-amino]-4-oxo-butanoic Acid Semicarbazone

To a solution of (2S-cis)-[5-benzoylamino-1,2,3,4,5,6,7-hexahydro-4-oxoazepino[3,2,1-hi]indole-2-carbonyl)-amino]-4-oxo-butanoic acid tert-butyl ester semicarbazone (0.064 g, 0.114 mmol) in methylene chloride (1 mL) was added anisole (0.5 mL) followed by trifluoroacetic acid (1 mL). After stirring at room temperature under nitrogen for 2.5 hours, the reaction mixture was diluted with ethyl acetate and evaporated to give the title compound (0.070 g). TLC (methylene chloride-methanol, 4:1) R_{f}=0.4. Mass spectrum: m/z 507 (M+H).

### EXAMPLE 100

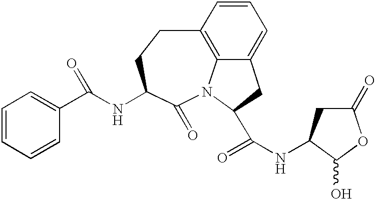

### (2S-cis)-[5-benzoylamino-1,2,3,4,5,6,7-hexahydro-4-oxoazepino[3,2,1-hi]indole-2-carbonyl)-amino]-4-oxo-butanoic Acid

(2S-cis)-[5-Benzoylamino-1,2,3,4,5,6,7-hexahydro-4-oxoazepino [3,2,1-hi]indole-2-carbonyl)-amino]-4-oxo-butanoic acid semicarbazone (0.070 g, ca. 0.114 mmol) was treated with a 3:1:1 solution of methanol-acetic acid-37% formaldehyde (3 mL), and the resulting mixture stirred under nitrogen for 3.5 hours. The reaction mixture was then diluted with water, methanol removed by evaporation, then the remaining mixture lyophilized. Purification of the crude product by flash chromatography on silica gel (S/P brand silica gel 60 Å, 230-400 mesh ASTM) eluting with 10 and 20% methanol-methylene chloride gave 0.042 g (82%) of the title compound as a white solid; m.p. 204-205° C. (dec). TLC (methylene chloride-methanol, 4:1) R_{f}=0.4. Mass spectrum: m/z 448 (M−H). ¹H-NMR (300 MHz, DMSO-d₆) δ 8.84 (m, 1H), 8.53 (m, 1H); 7.91-7.95 (m, 2H), 7.46-7.58 (m, 3H), 7.11 (m, 2H), 6.99 (t, J=7.3 Hz, 1H), 5.14 (d, 10.2 Hz, 1H), 4.62 (m, 1H), 4.23 (m, 1H), 3.48 (m, 1H), 3.12-3.18 (m, 2H), 2.99 (m, 1H), 2.58 (m, 1H), 2.12-2.46 (m, 3H).

### EXAMPLE 101

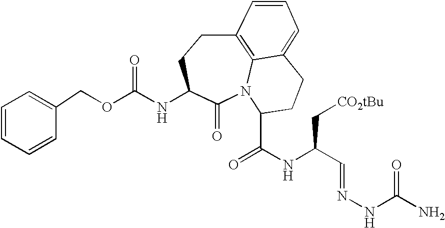

### (3R,S-cis)-6-benzyloxycarbonylamino-5-oxo-2,3,4,5,6,7,8-hexahydro-1H-azepino[3,2,1-hi]quinoline-3-carbonyl)-amino]-4-oxo-butanoic Acid Tert-Butyl Ester Semicarbazone

1. Preparation of (3R,S-cis)-6-Benzyloxycarbonylamino-5-oxo-2,3,4,5,6,7,8-hexahydro-1H-azepino[3,2,1-hi]quinoline-3-carboxylic Acid, Methyl Ester

To a solution of (3R,S-cis)-6-Amino-5-oxo-2,3,4,5,6,7,8-hexahydro-1H-azepino[3,2,1-hi]quinoline-3-carboxylic acid, methyl ester (0.570 g, 2.1 mmol, prepared as described in Tetrahderon Letters 36, pp. 1593-1596 (1995) and U.S. Pat. No. 5,504,080 (Apr. 2, 1996) in methylene chloride (6 mL) stirring at 0° C. was added benzyl chloroformate (0.6 mL, 4.2 mmol) and triethylamine (1.2 mL, 8.4 mmol) and the resulting mixture was stirred under nitrogen for 30 minutes. The reaction was quenched with water then partitioned between ethyl acetate and 5% aqueous potassium bisulfate solution. The aqueous layer was back extracted two times with ethyl acetate, then the combined organic layers were washed with saturated sodium chloride solution, dried (sodium sulfate) and evaporated to dryness. Purification of the crude product by flash chromatography on silica gel (S/P brand silica gel 60 Å, 230-400 mesh ASTM) eluting with ethyl acetate-hexane (2:1) gave 0.643 g (76%) of the title compound as a white foam. TLC (methylene chloride-methanol, 95:5) R_{f}=0.8. ¹H-NMR (300 MHz, CDCl₃) δ 7.36-7.25 (m, 5H), 7.13-7.02 (m, 3H), 5.67 (d, J=7.8 Hz, 1H), 5.02 (t, J=9.15, 18.3 Hz, 2H), 4.34 (m, 1H), 3.70 (s, 3H), 3.16 (m, 1H), 2.69-2.56 (m, 5H), 2.06 (m, 1H). Mass spectrum: m/z 408 (M+H).

2. Preparation of (3R,S-cis)-6-Benzyloxycarbonylamino-5-oxo-2,3,4,5,6,7,8-hexahydro-1H-azepino [3,2,1-hi]quinoline-3-carboxylic Acid

To a solution of (3R,S-cis)-6-Benzyloxycarbonylamino-5-oxo-2,3,4,5,6,7,8-hexahydro-1H-azepino[3,2,1-hi]quinoline-3-carboxylic acid, methyl ester (0.622 g, 1.53 mmol) in 1,4-dioxane (10.5 mL) and water (3.5 mL) was added 1M aqueous lithium hydroxide (2.3 mL, 2.3 mmol) and the resulting mixture was stirred at room temperature under nitrogen for 1 hour. The reaction mixture was acidified to ca. pH 2 with a 5% aqueous potassium bisulfate solution, then partitioned between ethyl acetate and saturated sodium chloride solution. The aqueous layer was back extracted two times with ethyl acetate, and the combined organic layers were dried (sodium sulfate) and evaporated to yield 0.670 g of the title compound. TLC (methylene chloride-methanol-acetic acid, 32:1:1) R_{f}=0.35. ¹H-NMR (300 MHz, CDCl₃): δ 7.38-7.28 (m, 5H), 7.13-7.04 (m, 3H), 5.72 (d, J=8.1 Hz, 1H), 5.03 (s, 2H), 4.35 (m, 1H), 3.77-3.67 (m, 5H), 3.10 (m, 1H), 2.72-2.52 (m, 5H), 2.07 (m, 1H), 1.70 (m, 1H).

3. (3R,S-cis)-6-Benzyloxycarbonylamino-5-oxo-2,3,4,5,6,7,8-hexahydro-1H-azepino [3,2,1-hi]quinoline-3-carbonyl)-amino]-4-oxo-butanoic Acid Tert-Butyl Ester Semicarbazone

To a solution of (3R,S-cis)-6-benzyloxycarbonylamino-5-oxo-2,3,4,5,6,7,8-hexahydro-1H-azepino[3,2,1-hi]quinoline-3-carboxylic acid (0.604 g, 1.5 mmol) in methylene chloride (12 mL) stirring at 0° C. under nitrogen was added 1-hydroxybenzotriazole hydrate (0.282 g, 1.8 mmol) and 1-(3-dimethylaminopropyl)-3-ethylcarbodiimide hydrochloride (0.442 g, 3 mmol). After 15 minutes, L-aspartic acid semicarbazone β-tert-butyl ester, p-toluenesulfonate salt (0.60 g, 1.5 mmol) and N-methylmorpholine (0.25 mL, 3 mmol) were added and the mixture allowed to come to room temperature within 1 hour. After stirring an additional hour, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60A, 230-400 mesh ASTM) eluting with 10% methanol-methylene chloride gave 0.523 g (56%) of the title compound as a white foam. TLC (methylene chloride-methanol, 9:1) R_{f}=0.65. ¹H-NMR (300 MHz, CDCl₃): δ 9.89 (m, 1H), 7.72 (m, 1H), 7.92 (d, J=9 Hz, 1H), 7.65 (d, J=8.1 Hz, 1H), 7.32-7.28 (m, 5H), 7.12 (s, 1H), 7.07 (d, J=5.7 Hz, 2H), 6.03 (d, J=7.5 Hz, 1H), 5.84 (d, J=8.1 Hz, 1H), 5.03 (s, 2H), 5.01 (m, 1H) 4.80 (m, 1H), 4.31 (m, 1H), 2.98 (m, 1H), 2.75-2.41 (m, 7H), 2.12 (m, 1H), 1.77 (m, 1H), 1.39 (s, 9H).

### EXAMPLE 102

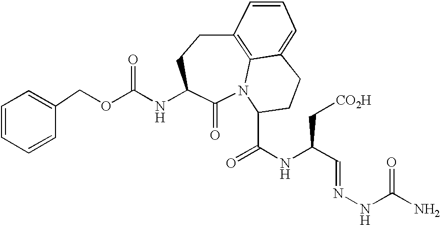

### (3R,S-cis)-6-benzyloxycarbonylamino-5-oxo-2,3,4,5,6,7,8-hexahydro-1H-azepino[3,2,1-hi]quinoline-3-carbonyl)-amino]-4-oxo-butanoic Acid Semicarbazone

To a solution of (3R,S-cis)-6-Benzyloxycarbonylamino-5-oxo-2,3,4,5,6,7,8-hexahydro-1H-azepino[3,2,1-hi]quinoline-3-carbonyl)-amino]-4-oxo-butanoic acid tert-butyl ester semicarbazone (0.200 g, 0.33 mmol) in methylene chloride (1 mL) was added anisole (0.5 mL, 4:62 mmol) followed by trifluoroacetic acid (1 mL). After stirring at room temperature under nitrogen for 1.5 hours the reaction mixture was diluted with methylene chloride and evaporated, then azeotroped twice with methylene chloride to give the title compound (0.248 g). TLC (methylene chloride-methanol-acetic acid, 8:1:1) R_{f}=0.2. Mass spectrum: m/z 549 [M−H]⁻.

### EXAMPLE 103

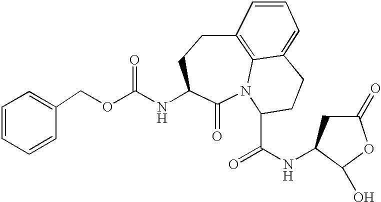

### (3R,S-cis)-6-benzyloxycarbonylamino-5-oxo-2,3,4,5,6,7,8-hexahydro-1H-azepino[3,2,1-hi]quinoline-3-carbonyl)-amino]-4-oxo-butanoic Acid

(3R,S-cis)-6-Benzyloxycarbonylamino-5-oxo-2,3,4,5,6,7,8-hexahydro-1H-azepino[3,2,1-hi]quinoline-3-carbonyl)-amino]-4-oxo-butanoic acid semicarbazone (0.245 g, ca 0.33 mmol), was treated with a 3:1:1 solution of methanol-acetic acid-37% formaldehyde (3 mL) and the resulting mixture stirred under nitrogen for 1.5 hours. The reaction mixture was diluted with water, methanol removed by evaporation, then the remaining mixture lyophilized. Purification of the crude product by flash chromatography on reverse phase gel (MCI gel, CHP-20P, 75-150 micron) eluting with a 10%-80% methanol-water gradient gave 0.090 g (60%) of the title compound as a white solid after lyophilization; m.p. 120-123° C. (dec). TLC (methylene chloride-methanol-acetic acid, 32:1:1) R_{f}=0.45. ¹H-NMR (300 MHz, DMSO d6): δ 8.67 (m, 1H), 7.79 (m, 1H), 7.57 (m, 1H), 7.37-7.27 (m, 5H), 7.17-7.08 (m, 3H), 5.44 (m, 1H), 4.95 (s, 2H), 4.70 (m, 1H), 4.07 (m, 1H), 3.92 (m, 1H), 3.16 (m, 1H), 2.98 (m, 1H), 2.75-2.41 (m, 7H), 2.25 (m, 1H), 2.11 (m, 1H), 1.29 (m, 1H). Mass spectrum: m/z 492 [M−H]⁻.

### EXAMPLE 104

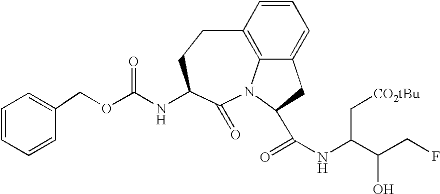

### 3 {(2S-cis)-[5-benzyloxycarbonylamino-1,2,3,4,5,6,7-hexahydro-4-oxoazepino[3,2,1-hi]indole-2-carbonyl)-amino]}-5-fluoro-4-hydroxy-pentanoic Acid Tert-Butyl Ester

To a solution of (2S-cis)-5-benzyloxycarbonylamino-1,2,3,4,5,6,7-hexahydro-4-oxoazepino[3,2,1-hi]indole-2-carboxylic acid (0.373 g, 0.98 mmol) in methylene chloride (3 mL) stirring at 0° C. under nitrogen was added 1-hydroxybenzotriazole hydrate (0.151 g, 0.98 mmol) and 1-(3-dimethylaminopropyl)-3-ethylcarbodiimide hydrochloride (0.283 g, 1.47 mmol). After 15 minutes, 3-amino-4-hydroxy-5-fluoropentanoic acid, tert-butyl ester (0.204 g, 0.98 mmol, prepared as described in Tetrahedron Letters 35, pp. 9693-9696 (1994)) was added and the mixture allowed to come to room temperature within 1 hour. After stirring overnight, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60 Å, 230-400 mesh ASTM) eluting with 2% methanol-methylene chloride gave 0.383 g (68%) of the title compound as a white foam. TLC (methylene chloride-methanol, 9:1) R_{f}=0.6. ¹H-NMR (300 MHz, CDCl₃): δ 7.45-7.31 (m, 5H), 7.08-7.01 (m, 3H), 6.10 (m, 1H), 5.26 (m, 1H), 5.12 (s, 2H), 4.52 (m, 1H), 4.38-4.30 (m, 2H), 4.21-4.19 (m, 2H), 4.03-3.95 (m, 2H), 3.43-3.20 (m, 4H), 3.13 (m, 2H), 2.62-2.50 (m, 2H), 2.42 (m, 1H), 1.42 (s, 4H), 1.32 (s, 5H). Mass spectrum: m/z 570 (M+H).

### EXAMPLE 105

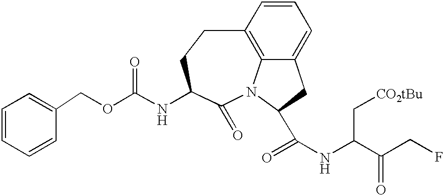

### 3 {(2S-cis)-[5-benzyloxycarbonylamino-1,2,3,4,5,6,7-hexahydro-4-oxoazepino[3,2,1-hi]indole-2-carbonyl)-amino]}-5-fluoro-4-oxo-pentanoic Acid Tert-Butyl Ester

To a solution of 3{(2S-cis)-[5-benzyloxycarbonylamino-1,2,3,4,5,6,7-hexahydro-4-oxoazepino[3,2,1-hi]indole-2-carbonyl)-amino]}-5-fluoro-4-hydroxy-pentanoic acid tert-butyl ester (0.114 g, 0.20 mmol) in methyl sulfoxide (1.3 mL) was added Dess-Martin periodinane (0.228 g). After stirring at room temperature under nitrogen for 2 hours an additional portion of Dess-Martin periodinane (0.135 g) was added followed 2.5 hours later by a third portion (0.10 g). The reaction mixture was diluted with ethyl acetate and washed twice with water and saturated sodium chloride solution; dried (sodium sulfate) and evaporated to dryness. Purification of the crude product by flash chromatography on silica gel (S/P brand silica gel 60 Å, 230-400 mesh ASTM) eluting 1/1 ethyl acetate-hexanes gave 0.076 g (67%) of the title compound as a white foam. TLC (ethyl acetate-hexanes, 1:1) R_{f}=0.6. ¹H-NMR (300 MHz, CDCl₃): δ 7.58 (d, J=8.4 Hz, 1H), 7.34-7.30 (m, 5H), 7.07-6.99 (m, 3H), 6.06 (m, 1H), 5.23 (d, J=12.3 Hz, 1H), 5.12 (s, 2H), 4.53 (d, J=13.2 Hz, 1H), 4.77 (d, J=9.9 Hz, 2H), 4.32 (m, 1H), 3.44 (dd, J=5, 8.4 Hz, 1H), 3.32-3.21 (m, 2H), 3.06 (m, 1H), 2.9 (m, 1H), 2.62 (m, 1H), 2.41 (m, 1H), 2.17 (m, 1H), 1.39 (s, 4H), 1.29 (s, 5H).

### EXAMPLE 106

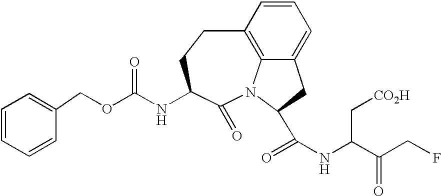

### 3 {(2S-cis)-[5-benzyloxycarbonylamino-1,2,3,4,5,6,7-hexahydro-4-oxoazepino [3,2,1-hi]indole-2-carbonyl)-amino]}-5-fluoro-4-oxo-pentanoic Acid

To a solution of 3 {(2S-cis)-[5-benzyloxycarbonylamino-1,2,3,4,5,6,7-hexahydro-4-oxoazepino [3,2,1-hi] indole-2-carbonyl)-amino]}-5-fluoro-4-oxo-pentanoic acid tert-butyl ester (0.063 g, 0.111 mmol) in methylene chloride (1.0 mL) was added anisole (0.5 mL), followed by trifluoroacetic acid (1.0 mL). After stirring at room temperature under nitrogen for 2 hours the reaction mixture was diluted with methylene chloride and evaporated, then chased twice with methylene chloride. The crude residue was triturated with ethyl ether to give 0.030 g of the titled product as a white solid; m.p. 106-107° C. (dec). TLC (methylene chloride-methanol-acetic acid, 32:1:1) R_{f}=0.3. ¹H-NMR (300 MHz, CDCl₃): δ 7.61 (m, 1H), 7.32 (s, 5H), 7.1 (d, J=4 Hz, 1H), 7.03 (d, J=4 Hz, 2H), 6.17 (m, 1H), 5.22 (m, 1H), 5.10 (s, 2H), 4.75-4.70 (m, 2H), 4.32 (m, 1H), 3.5 (m, 1H), 3.31-3.15 (m, 2H), 3.03 (m, 1H), 2.93 (m, 1H), 2.69 (m, 1H), 2.36 (m, 1H), 2.12 (m, 1H). Mass spectrum: m/z 512 (M+H).

### EXAMPLE 107

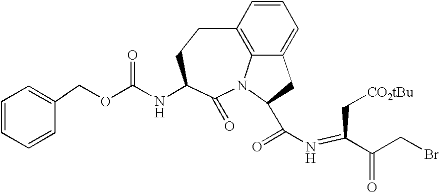

### 3-[(2S-cis)-(5-benzyloxycarbonylamino-1,2,3,4,5,6,7-hexahydro-4-oxoazepino[3,2,1-hi]indole-2-carbonyl)amino]-5-bromo-4-oxo-pentanoic Acid, Tert-Butyl Ester

To a solution of (2S-cis)-5-benzyloxycarbonylamino-1,2,3,4,5,6,7-hexahydro-4-oxoazepino[3,2,1-hi]indole-2-carboxylic acid (0.302 g, 0.797 mmol) in methylene chloride (5.5 mL) stirring at 0° C. under nitrogen was added 1-hydroxybenzotriazole hydrate (0.146 g, 0.96 mmol) and 1-(3-dimethylaminopropyl)-3-ethylcarbodiimide hydrochloride (0.230 g, 1.2 mmol). After 15 minutes, aspartic acid, α-methyl, β-tert-butyl diester hydrochloride (0.191 g, 0.797 mmol) was added followed by N-methylmorpholine (0.13 mL, 1.2 mmol) and the mixture allowed to come to room temperature within 1 hour. After stirring overnight, the reaction mixture was diluted with ethyl acetate and washed successively with 5% potassium bisulfate and saturated sodium chloride solutions; dried (sodium sulfate) and evaporated to dryness. Purification of the crude product by flash chromatography on silica gel (S/P brand silica gel 60 Å, 230-400 mesh ASTM) eluting with ethyl acetate-hexane (1:1) gave 0.350 g (78%) of N-[(2S-cis)-[5-benzyloxy-carbonylamino-1,2,3,4,5,6,7-hexahydro-4-oxoazepino[3,2,1-hi]indole-2-carbonyl]]aspartic acid, α-methyl, β-tert-butyl diester as a white solid. TLC (methylene chloride-methanol, 9:1) R_{f}=0.8. m.p. 147-148° C. (dec.). ¹H-NMR (300 MHz, CDCl₃): δ 7.48 (d, J=7.5 Hz, 1H), 7.34-7.29 (m, 5H), 7.07 (m, 1H), 7.03-6.96 (m, 2H), 6.15 (d, J=5.7 Hz, 1H), 5.28 (d, J=7.8 Hz 1H), 5.11 (s, 2H), 4.72 (m, 1H), 4.32 (m, 1H), 3.74 (s, 3H), 3.49 (d, J=16.5 Hz, 1H), 3.31-3.20 (m, 2H), 3.05 (m, 1H), 2.72 (ABX, dd, J=4.65, 15, 64.5 Hz, 2H), 2.43 (m, 1H), 2.15 (m, 1H), 1.30 (s, 9H).

To a solution of the above product (0.330 g, 0.585 mmol) in 1,4-dioxane (4.5 mL) and water (1.5 mL) was added 1M aqueous lithium hydroxide (0.7 mL, 0.702 mmol) and the resulting mixture was stirred at room temperature under nitrogen for 30 minutes. The reaction mixture was acidified to pH 3 with a 0.1N HCl solution, then partitioned between ethyl acetate and saturated sodium chloride solution. The aqueous layer was back extracted two times with ethyl acetate, and the combined organic layers were dried (sodium sulfate) and evaporated to yield 0.275 g (85%) of N-[(2S-cis)-[5-benzyloxycarbonylamino-1,2,3,4,5,6,7-hexahydro-4-oxoazepino[3,2,1-hi]indole-2-carbonyl]]aspartic acid, β-tert-butyl ester as a white foam. TLC (methylene chloride-methanol, 9:1): R_{f}=0.25. ¹H-NMR (300 MHz, CDCl₃): d 7.57 (d, J=7.8 Hz, 1H), d 7.35-7.29 (m, 5H), 7.08 (m, 1H), 7.03-6.98 (m, 2H), 6.24 (d, J=6 Hz, 1H), 5.28 (d, J=5.1 Hz, 1H), 5.11 (s, 2H), 4.73 (m, 1H), 4.35 (m, 1H), 3.48 (d, J=16.8 Hz, 1H), 3.36-3.20 (m, 2H), 3.07 (m, 1H), 2.76 (ABX, dd, J=4.8, 18, 66 Hz, 2H), 2.40 (m, 1H), 2.19 (m, 1H), 1.33 (s, 9H).

To a solution of the above product (0.262 g, 0.475 mmol) in tetrahydrofuran (3.0 mL) stirring at −10° C. under nitrogen was added N-methylmorpholine (0.114 mL, 1.05 mmol) followed by dropwise addition of isobutyl chloroformate (0.107 mL, 0.81 mmol). After 40 minutes the reaction mixture was filtered, the salts washed with dry THF, and the filtrate cooled to 0° C. This was treated with a freshly prepared ethereal solution of diazomethane (excess). After stirring the mixture at 0° C. for 30 minutes, a mixture of hydrobromic acid (48% wt. aq. solution)/acetic acid (1.3 mL, 1/1) was added dropwise. After stirring for another 10 minutes, the reaction mixture was diluted with ethyl acetate, then washed successively with saturated sodium bicarbonate and saturated sodium chloride solutions; dried (sodium sulfate) and evaporated to dryness. Purification of the crude product by flash chromatography on silica gel (S/P brand silica gel 60 Å, 230-400 mesh ASTM) eluting with ethyl acetate-hexane (1:1) gave 0.200 g (67%) of the title compound as a white foam. TLC (ethyl acetate-hexane (1:1): R_{f}=0.7. ¹H-NMR (300 MHz, CDCl₃): δ 7.71 (d, J=9 Hz, 1H), d 7.35-7.30 (m, 5H), 7.09 (m, 1H), 7.04-7.02 (m, 2H), 6.1 (d, J=5.4 Hz, 1H), 5.28 (d, J=7.2 Hz, 1H), 5.12 (s, 2H), 4.89 (dd, J=4.5, 15 Hz, 1H), 4.35 (m, 1H), 4.16 (s, 2H), 3.50-3.21 (m, 3H), 3.06 (m, 1H), 2.76 (ABX, dd, J=4.65, 18, 103 Hz, 2H), 2.37 (m, 1H), 2.15 (m, 1H), 1.27 (s, 9H). Mass spectrum: m/z 626/628 (M−H).

### EXAMPLE 108

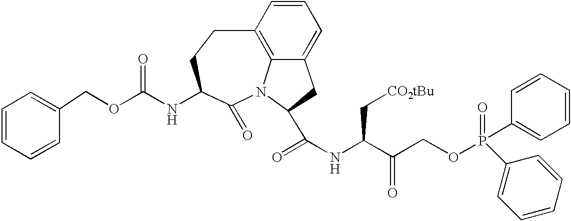

### 3-[(2S-cis)-[5-benzyloxycarbonylamino-1,2,3,4,5,6,7-hexahydro-4-oxoazepino[3,2,1-hi]indole-2-carbonyl]amino]-5-(diphenylphosphinyl)oxy-4-oxo-pentanoic Acid, Tert-Butyl Ester

To a solution of 3-[(2S-cis)-[5-benzyloxycarbonylamino-1,2,3,4,5,6,7-hexahydro-4-oxoazepino [3,2,1-hi] indole-2-carbonyl]amino]-5-bromo-4-oxo-pentanoic acid, tert-butyl ester (0.069 g, 0.110 mmol) in N,N-dimethylformamide (1.0 mL) was added potassium fluoride (0.029 g, 0.495 mmol), followed diphenylphosphinic acid (0.029 g, 0.139 mmol). After stirring at room temperature under nitrogen for 48 hours, the reaction mixture was diluted with ethyl acetate, then washed successively with a dilute sodium bicarbonate solution then water; dried (sodium sulfate) and evaporated to dryness. Purification of the crude product by flash chromatography on silica gel (S/P brand silica gel 60 Å, 230-400 mesh ASTM) eluting with ethyl acetate-hexane (1:1) gave 0.048 g (59%) of the title compound as a clear oil. TLC (ethyl acetate-hexane, 2:1): R_{f}=0.3. ¹H-NMR (300 MHz, CDCl₃): δ 7.89-7.80 (m, 4H), 7.52-7.30 (m, 11H), 7.06 (m, 1H), 7.01-6.96 (m, 2H), 6.45 (m, 1H), 5.21 (m, 1H), 5.13 (s, 2H), 4.96 (dd, J=8.3, 18 Hz, 1H), 4.78-4.70 (m, 2H), 4.35 (m, 1H), 3.35-3.23 (m, 3H), 3.05 (m, 1H), 2.76 (ABX, dd, J=4.65, 18, 103 Hz, 2H), 2.43 (m, 1H), 2.18 (m, 1H), 1.33 (s, 9H).

### EXAMPLE 109

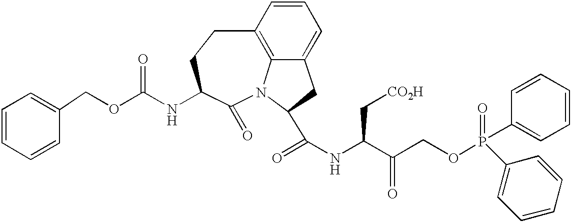

### 3-[(2S-cis)-[5-benzyloxycarbonylamino-1,2,3,4,5,6,7-hexahydro-4-oxoazepino[3,2,1-hi]indole-2-carbonyl]amino]-5-(diphenylphosphinyl)oxy-4-oxo-pentanoic Acid

To a solution of 3-[(2S-cis)-[5-benzyloxycarbonylamino-1,2,3,4,5,6,7-hexahydro-4-oxoazepino[3,2,1-hi]indole-2-carbonyl]amino]-5-(diphenylphosphinyl)oxy-4-oxo-pentanoic acid, tert-butyl ester (0.040 g, 0.054 mmol) in methylene chloride (1.0 mL) was added anisole (0.5 mL), followed by trifluoroacetic acid (1.0 mL). After stirring at room temperature under nitrogen for 30 minutes the reaction mixture was diluted with methylene chloride and evaporated, then azeotroped twice with methylene chloride. The crude residue was triturated with ethyl ether to give 0.030 g of the titled product as a white solid; m.p. 109-111° C.(dec). TLC (methylene chloride-methanol, 9:1): R_{f}=0.4. ¹H-NMR (300 Mhz, CDCl₃): δ 7.87-7.66 (m, 4H), 7.60-7.28 (m, 11H), 7.05-6.95 (m, 3H), 6.84 (m, 1H), 5.12 (s, 2H), 5.05 (m, 1H), 4.58 (m, 1H), 4.42-4.15 (m, 4H), 3.35-3.10 (m, 4H), 3.05 (m, 1H), 2.76 (m, 1H), 2.56 (m, 1H), 2.37 (m, 1H), 2.13 (m, 1H), 1.93 (bs, 1H). Mass spectrum: m/z 710 (M+H).

### EXAMPLE 110

### Materials and Methods for Evaluating Effects of ICE/CED-3 Inhibitors on Granulocyte Neutrophils

Neutrophil Isolation:

Whole blood anticoagulated with Acid Citrate Dextrose (ACD) with a ratio of 1:5 ACD to blood was collected (˜100 ml).

Using polypropylene plastic ware, neutrophils are isolated as follows:

30 ml of the whole blood is added to 50 ml polypropylene centrifuge tubes containing 15 ml of 6% Dextran (in Saline). The blood is allowed to sediment for approximately 1 hour at room temperature.

The turbid straw colored layer harvested from the top of the cylinders into 50 ml conical polypropylene tubes. The blood cells were pelleted by centrifugation at 240× g (Sorvall centrifuge at 1200 rpm) for 12 min. at 4° C. with the brake on low.

The supernatant was aspirated and the pooled pellet resuspended in 40-50 ml cold PBS (w/o Ca, Mg), and centrifuged at 240× g (Sorvall centrifuge at 1200 rpm) for 6 min. at 4° C. with the brake on high.

The supernatant was aspirated and the pellet resuspended in 12 ml of cold cell culture grade water. The suspension was titriated gently with a pipet for 30 seconds then add 4 ml of cold 0.6 M KCl. (Brought up to 50 ml with cold PBS (w/o Ca, Mg)) and then centrifuged at 300× g (Sorvall centrifuge at 1400 rpm) for 6 min. at 4° C. with the brake on high.

The above was repeated onetime.

The supernatant was aspirated and the cells resuspended in 2.5 ml cold PBS (w/o Ca, Mg). The cell suspension was layered over 3 ml Ficoll-Hypaque in a 15 ml polypropylene conical tube and centrifuged at 400× g (Sorvall centrifuge at 1900 rpm) for 30 min. at 4° C. with the brake on low.

The suspension aspirated was down to the neutrophil pellet. The pellet was resuspended in cold PBS (w/o Ca, Mg) and transferred to a 50 ml conical tube and brought to 50 ml with cold PBS (w/o Ca, Mg) and centrifuged at 300× g (Sorvall centrifuge at 1400 rpm) for 6 min. at 4° C. with the brake on high.

The supernatant was aspirated and the pellet resuspended in 50 ml cold PBS (w/o Ca, Mg) and centrifuged at 300× g (Sorvall centrifuge at 1400 rpm) for 6 min. at 4° C. with the brake on high.

The supernatant was aspirated and the neutrophil pellet resuspended in 4.0 ml cold PBS (w/o Ca, Mg) on ice. 10 μl of the neutrophil cell suspension was diluted with 990 μl of Trypan blue (1:100) and cells counted using a hemacytometer. The cell number and viability were determined.

Neutrophil Culture Conditions:

The culture media was as follows: (RPMI 1640; 10% FBS; 10 mM Hepes; 0.2 mM L-glutamine; 25 U/ml penicillin; and 25 mg/ml streptomycin).

Purified neutrophil maintenance was performed under the following conditions: (5×10⁶ cells/ml in above culture media; Polystyrene round-bottom 96-well plates; 250 μl/well; and 37° C., 5% CO₂/95% air humidified incubator) (Liles et al., Blood 119 (1995) 3181-3188).

Analysis of Hypodiploid Nuclei by Flow Cytometry:

Hypotonic Fluorochrome Solution

(50 μg/ml propidium iodide (Sigma catalog#P4170); 0.1% Triton X-100; and 0.1 sodium citrate).

Neutrophils were pelleted at 4° C. and the supernate aspirated.

Neutrophils were resuspended in hypotonic fluorochrome solution at a density of 5×10⁶ cells/ml. Propidium iodide fluorescence of individual nuclei was evaluated in FL2 and measured on a logarithmic scale while gating on physical parameters in forward and side scatter to exlude cell debris.

At least 10,000 events per sample were collected and the results were evaluated relative to a non-apoptotic neutrophil population. (Liles et al., Blood 119 (1995) 3181-3188).

Respiratory Burst in Isolated Neutrophils Measured by Chemiluminescence

Whole blood anticoagulated with Acid Citrate Dextrose (ACD) with a ratio of 1:5 ACD to blood was collected (150 ml).

Neutrophils were isolated as described above.

Opsonized zymosan was prepared by suspending 125 mg zymosan particles in 25 ml pooled human serum (5 mg/ml) and incubating them for 20 minutes at 37° C. Centrifuge the suspension and resuspend the particles in 7 ml of PBS (18 mg/ml) and stored on ice until use (vortex prior to pipetting).

50 ml of a 250 μM solution of Lucigenin (MW 510.5) was prepared by dissolving 6.4 mg of the solid in 50 ml of PBS-G (+Ca, Mg). 10 μl of PBS-G (+Ca, Mg) to the wells in a white 96 well plate.

50 μl of the 250 μM Lucigenin solution was added to the wells in a white 96 well plate.

Cell preparations were obtained from cell culture (concentration at time zero=5.0×10⁶ cells/ml) with PBS-G (+Ca, Mg).

20 μl of the neutrophil suspension was suspended to the appropriate wells and the plate was incubated at 37° C. for three minutes. 10 μl of the opsonized zymosan was added to the wells.

The plate was read on the luminometer (Labsystems Luminoskan, Needham Heights, Mass.) for 14 min. at 37° C. in the kinetic mode and record results using the software DeltaSoft.

Whole Blood Assay:

The following reagents were used:

anti-CD32-FITC monoclonal antibody obtained from Pharmingen.

Lysing Solution (10× Stock: 89.9 g NH4C1;

10.0 g KHCO;

0.37 g tetrasodium EDTA;

dissolve in 1 liter dH2O. Adjust to pH 7.3. Store at 4° C. in a tightly closed bottle.

Dilute 1:10 with dH2O prior to use.)

(DPBS without calcium or magnesium obtained from Irivine Scientific.

2% fetal bovine serum in DPBS stored at 4° C.

50 μg/ml propidium iodide in DPBS sterile filtered and stored at 4° C.)

The following protocol was followed:

200 μl blood sample/2.8 ml 1× lysis solution in a 15 ml polypropylene conical tube.

Cap and invert to mix. Leave at room temperature for 3-5 minutes.

Centrifuge in a table-top Sorvall at 1200 rpm for 5 minutes at 4° C.

Aspirate supernate. Resuspend pellet in 200 μl/sample 2% FBS/DPBS.

Add 20 μl/sample anti-CD32-FITC. Incubate 30 minutes on ice in the dark.

Add 5 ml/sample DPBS. Centrifuge at 1000 rpm for 5 minutes at 4° C.

Aspirate supernate. Resuspend pellet in 1 ml/sample 2% FBS/DPBS.

Add 3 ml/sample ice-cold 95% EtOH dropwise while vortexing gently.

Incubate samples on ice in the dark for 30 minutes.

Centrifuge at 1000 rpm for 5 minutes at 4° C.

Resuspend each sample in 50 μl 5 mg/ml RN'ase. Transfer sample to 900 μl/sample.

50 μg/ml Propidium Iodide in 12×75 mm Falcon polystyrene tubes.

Incubate on ice for 30 minutes.

Analyze samples by flow cytometry (argon laser) for forward and side scatter and fluorescence.

### EXAMPLE 111

### Enhancement of Neutrophil/Granulocyte Survival by ex-vivo Application of ICE/CED-3 Inhibitors

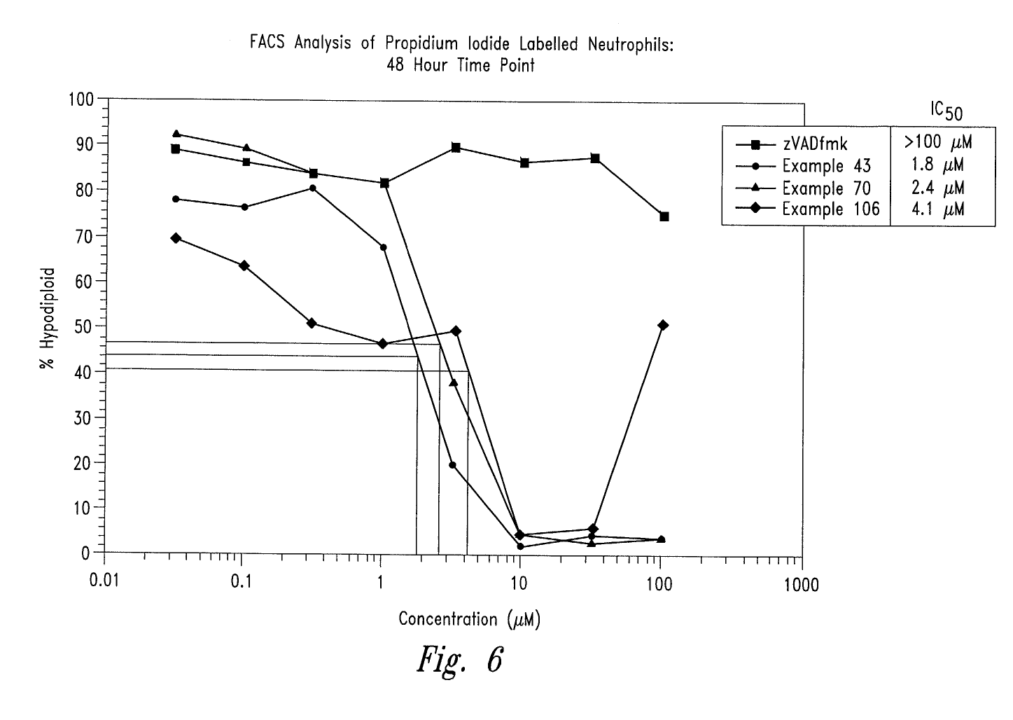

The present invention provides methods to enhance the ex vivo survival of neutrophils/granulocytes. To establish the ability of compounds to preserve granulocytes in culture, compounds were tested in a number of in vitro assays. One common model to test for effects on granulocyte survival involves separating granulocytes from fresh whole blood, culturing the cells at 37° C. and testing cells for nuclear hypodiploidy at 24 hour intervals (as described in Example 110). The presence of hypodiploid DNA is a measure of apoptosis, and is assessed using a propidium iodide stain via flow cytometry. Compounds of the present invention were incubated with the granulocytes in culture, their effects on granulocyte survival measured, and an IC50 calculated. In FIG. 6, the caspase inhibitor zVADfink prepared as described in Tetrahedron Letter, 35 9693-9696 (1994) had a weak effect on improving granulocyte survival at 48 hours, whereas examples 43, 70, and 106 from the present invention had IC50s of <5 μM and thus are potent inhibitors of granulocyte death.

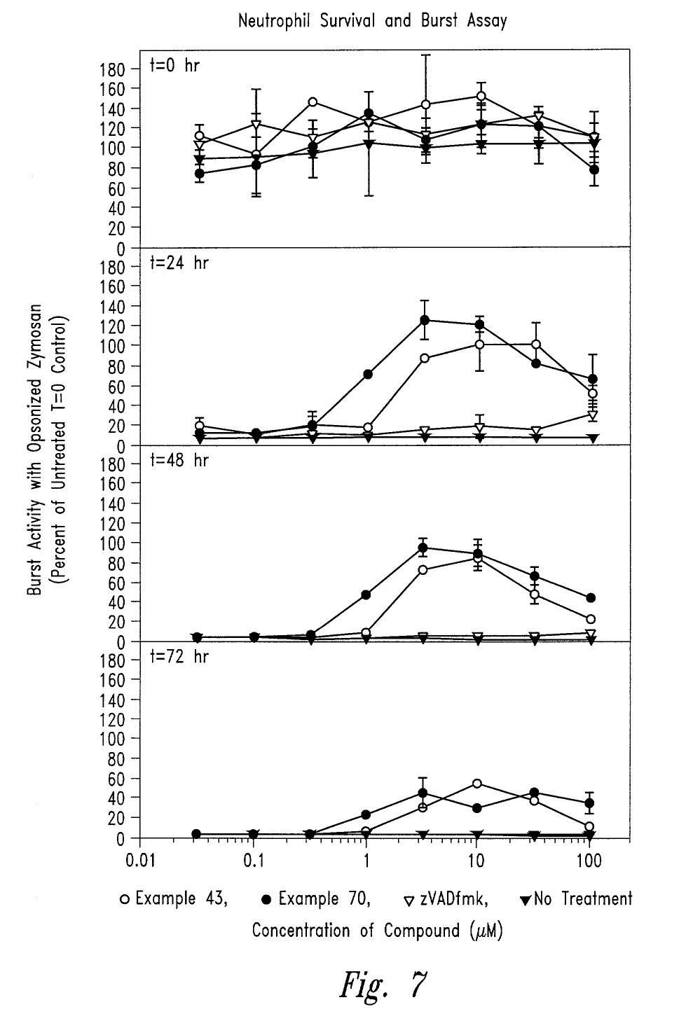

The ability to undergo the respiratory burst is another measure of granulocyte viability. The respiratory burst is a physiological response of granulocytes to foreign stimuli such as bacteria. In this example, the method for inducing the respiratory burst utilized opsonized bacterial zymosan. The respiratory burst was measured via chemiluminescence. FIG. 7 shows that the caspase inhibitor zVADfmk, which had only weak effects on the viability of the granulocytes in the hypodiploidy experiments, did not maintain the respiratory burst. In contrast, two exemplary compounds of the present invention, example 43 and 70, substantially maintained the respiratory burst for 48 hours, and partially maintained the respiratory burst after granulocyte culture for 72 hours.

Survival of granulocytes in whole blood was measured by hypodiploid analysis in a similar fashion to isolated granulocytes via flow cytometry. ICE/ced-3 inhibitors of the present invention maintained survival of granulocytes in whole blood for 96 hours at room temperature as indicated in the table below:

Percentage of diploid granulocytes in whole blood

| | time zero | 96 hours |
|---|---|---|
| no compound | 96% | 48% |
| EXAMPLE 43 | 96% | 91% |
| EXAMPLE 70 | 96% | 89% |

Thus, the present invention provides methods for maintaining the ex vivo survival of mature granulocytes, both isolated and in whole blood. The methods of this example also provide a means to distinguish those ICE/ced-3 inhibitors that are effective in maintaining granulocyte survival from those that are not effective.

### EXAMPLE 112

### Enhancement of Apheresis Product Survival by Application of ICE/CED-3 Inhibitors

Apheresis (leukapheresis) of blood donors can be performed to obtain a population of cells which is enriched in granulocytes. These cells are then transfused into a recipient in need of additional granulocytes. This apheresis product has a short shelf life, and current standards (American Association of Blood Banks, Standard for Blood Banks and Transfusion Services, Ed. 17, 1996) require storage at 20-24° C. for no longer than 24 hours. Transfusion is recommended within 6 hours if possible.

Exemplary compounds as described in the present invention can be used to prolong the storage life of apheresis products. ICE/ced-3 inhibitors are effective in prolonging granulocyte survival as shown in Example 64 for isolated granulocytes and whole blood. For use in the setting of apheresis, the compound can be formulated in a compatible solvent, such as dimethyl sulfoxide (DMSO). The compound can be stored in a vial, and be pre-added to the apheresis bag, or injected into the donor apheresis line during the collection process. The effective final concentration compound could range from 1-25 μM. The leukapheresis product, containing the ICE/ced-3 inhibitor, is then infused into the recipient after storage. Many storage conditions may be possible, for example, storage may be at room temperature for up to one week post-collection.

### EXAMPLE 113

### CMV PP65 Antigenicity Enhancement

Cytomegalovirus (CMV) antigenemia assay is the method of choice for rapid quantitative diagnosis of CMV infection and monitoring antiviral therapy. Due to the rapid loss of infected neutrophils by apoptosis, specimens must be processed within 6 hr of collection. Processing after 6 hr may diminish pp65 positive cell counts, leading to potentially erroneous values and quantitative levels for the patients' risk of CMV disease.

The study objective was to determine if pp65 antigenicity of CMV infected peripheral blood leukocytes (PBLs) could be preserved by the addition of a compound of the following formula:

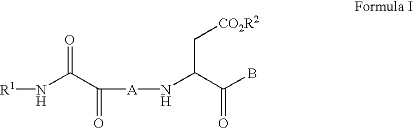

wherein:

A is a natural or unnatural amino acid of Formula IIa-i:

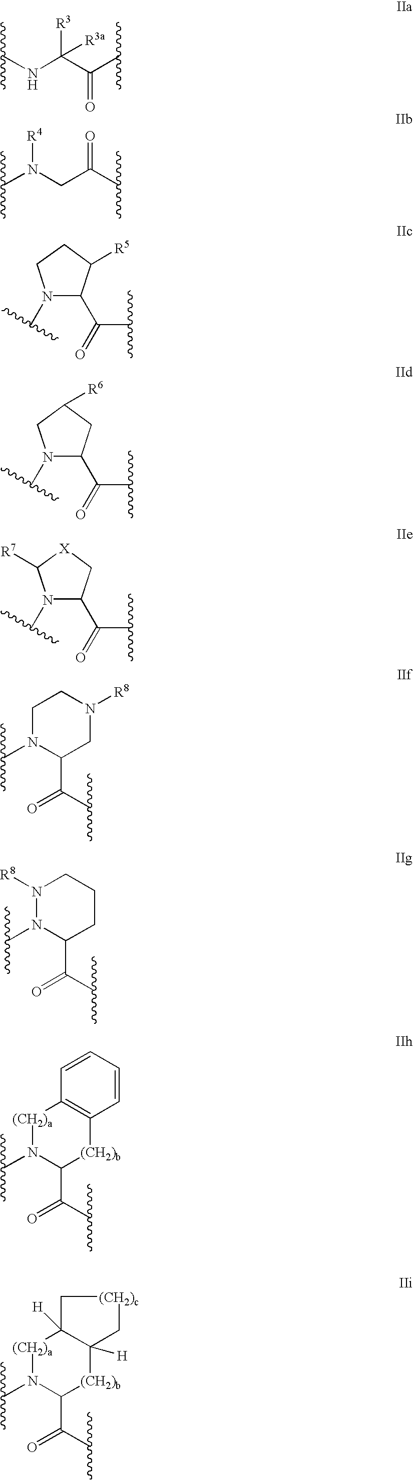

B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₙcycloalkyl, (CH₂)ₙphenyl, (CH₂)ₙ(substituted phenyl), (CH₂)ₙ(1 or 2-naphthyl), (CH₂)ₙ(heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c:

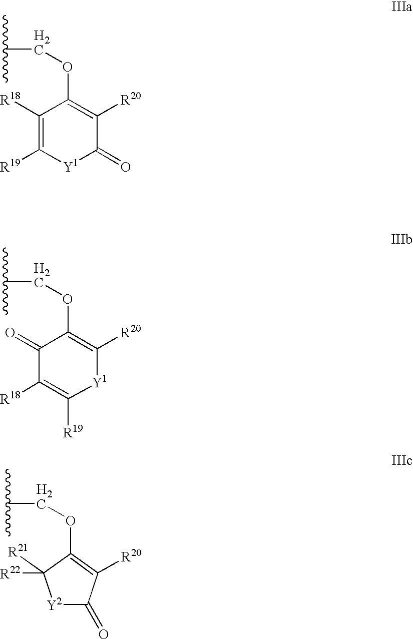

R¹ is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, (heteroaryl)alkyl, R^{1a}(R^{1b})N, [or] R^{1c}O, 2-phenoxyphenyl or 2- or 3-benzylphenyl; and

R² is hydrogen, lower alkyl, cycloalkyl, (cycloalkyl)alkyl, phenylalkyl, or substituted phenylalkyl;

and wherein:

R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl, with the proviso that R^{1a} and R^{1b} cannot both be hydrogen;

R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl;

R³ is C₁₋₆ lower alkyl, cycloalkyl, phenyl, substituted phenyl, (CH₂)ₙNH₂, (CH₂)ₙNHCOR⁹, (CH₂)ₙN(C═NH)NH₂, (CH₂)ₘCO₂R², (CH₂)ₘOR¹⁰, (CH₂)ₘSR¹¹, (CH₂)ₙcycloalkyl,(CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;

R^{3a} is hydrogen or methyl, or R³ and R^{3a} taken together are —(CH₂)_{d}— where d is an integer from 2 to 6;

R⁴ is phenyl, substituted phenyl, (CH₂)ₘphenyl, (CH₂)ₘ(substituted phenyl), cycloalkyl, or benzofused cycloalkyl;

R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);

R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹ or NHCOR⁹;

R⁷ is hydrogen, oxo, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;

R⁹ is hydrogen, lower alkyl, cycloalkyl, phenyl, substituted phenyl, naphthyl, (CH₂)ₙcycloalkyl, (CH₂)ₙphenyl, 12 (CH₂)ₙ(substituted phenyl), (CH₂)ₙ(1 or 2-naphthyl), OR¹², or NR¹³R^{14;}

R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);

R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);

R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);

R¹⁴ is hydrogen or lower alkyl;

or R¹³ and R¹⁴ taken together form a five to seven membered carbocyclic or heterocyclic ring, such as morpholine, or N-substituted piperazine;

R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);

R¹⁶ and R¹⁷ are independently lower alkyl, cycloalkyl, phenyl, substituted phenyl, naphthyl, phenylalkyl, substituted phenylalkyl, or (cycloalkyl)alkyl;

R¹⁸ and R¹⁹ are independently hydrogen, alkyl, phenyl, substituted phenyl, (CH₂)ₙphenyl, (CH₂)ₙ(substituted phenyl), or R's and R¹⁹ taken together are —(CH═CH)₂—;

R²⁰ is hydrogen, alkyl, phenyl, substituted phenyl, (CH₂)ₙphenyl, (CH₂)ₙ(substituted phenyl);

R²¹, R²², and R²³ are independently hydrogen, or alkyl;

X is CH₂, (CH₂)₂, (CH₂)₃, or S;

Y¹ is O or NR²³;

Y² is CH₂, O, or NR²³;

a is 0 or 1 and b is 1 or 2, provided that when a is 1 then b is 1;

c is 1 or 2, provided that when c is 1 then a is 0 and b is 1;

m is 1 or 2; and

n is 1,2, 3 or 4;

or a pharmaceutically acceptable salt thereof.

Eighteen bone marrow transplant patients with suspected active CMV infection were investigated. For each sample, a 10 mL peripheral blood specimen was split and the apoptotic inhibitor added to half the sample. The other half was used as a control. Aliquots were taken at 0 hr, 48 hr and 72 hr and enriched for PBLs by dextran separation followed by RBC lysis. Two slides were prepared by cytocentrifugation for each condition. Each slide was stained for the pp65 CMV early immediate antigen using a brightfield immunocytochemical staining method. Slides were then analyzed using the Automated Cellular Imaging System (ACIS™), reviewed and the number of pp65 positive cells determined. A Wilcoxan Matched Pairs test was applied to the data.

No significant differences in the number of pp65 cells, detected by the ACIS were found between the freshly-drawn (0 hr) and either the 48 hr (n=18, p=0.14) or 72 hr (n—14, p=0.48) samples in the presence of the apoptotic inhibitor. In the absence of the inhibitor, a significant reduction in pp65—positive cells was observed at both later timepoints.

Accordingly, apoptotic inhibitors preserve pp65—antigen positivity through 72 hr. Increasing sample stability to 72 hr results in a more robust CMV antigenemia assay adaptable to centralized laboratories and more accurate assessment of CMV active infections and disease management.

Although the invention has been described with reference to the examples provided above, it should be understood that various modifications can be made without departing from the spirit of the invention. Accordingly, the invention is limited only by the claims.

From the foregoing it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for preserving antigen presentation on a virally infected mammalian cell, comprising:
(a) providing a population of mammalian cells at least a portion of which is suspected of being virally infected and
(b) contacting said cells with an anti-apoptotic reagent, thereby preserving antigen presentation on virally infected cells.

2. The method of claim 1, wherein said cells comprise peripheral blood leukocytes.

3. The method of claim 1, wherein said cells comprise neutrophils.

4. The method of claim 1, wherein said cells comprise granulocytes.

5. The method of claim 1, wherein said virus is selected from the group consisting of herpes, HIV, cytomegalovirus (CMV), and hepatitis.

6. The method of claim 5, wherein said virus is CMV.

7. The method of claim 1, wherein said antigen comprises a viral antigen present on the surface of said mammalian cells.

8. The method of claim 7, wherein said antigen comprises pp65 protein of CMV.

9. The method of claim 1, wherein the contacting is ex vivo.

10. The method of claim 1, wherein the reagent is a nucleic acid.

11. The method of claim 10, wherein the reagent is an ICE antisense sequence.

12. The method of claim 1, wherein the reagent is a protease inhibitor.

13. The method of claim 12, wherein the protease inhibitor is irreversible.

14. The method of claim 12, wherein the protease inhibitor is reversible.

15. The method of claim 12, wherein the protease inhibitor is a compound of formula I: 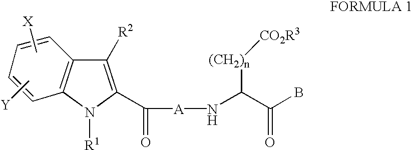
wherein:
n is 1 or 2;.
R¹ is alkyl, cycloalkyl, (cycloalkyl)alkyl, phenyl, (substituted)phenyl, phenylalkyl, (substituted)phenylalkyl, heteroaryl, (heteroaryl)alkyl or (CH₂)ₘCO₂R⁴, wherein m=1-4, and R⁴ is as defined below;
R² is a hydrogen atom, chloro, alkyl, cycloalkyl, (cycloalkyl)alkyl, phenyl, (substituted)phenyl, phenylalkyl, (substituted)phenylalkyl, heteroaryl, (heteroaryl)alkyl or (CH₂)ₚCO₂R⁵, wherein p=0-4, and R⁵ is as defined below;
R³ is a hydrogen atom, alkyl, cycloalkyl, (cycloalkyl)alkyl, phenylalkyl, or (substituted)phenylalkyl;
R⁴ is a hydrogen atom, alkyl, cycloalkyl, (cycloalkyl)alkyl, phenylalkyl, or (substituted)phenylalkyl;
R⁵ is a hydrogen atom, alkyl, cycloalkyl, (cycloalkyl)alkyl, phenylalkyl, or (substituted)phenylalkyl;
A is a natural and unnatural amino acid;
B is a hydrogen atom, a deuterium atom, alkyl, cycloalkyl, (cycloalkyl)alkyl, phenyl, (substituted)phenyl, phenylalkyl, (substituted)phenylalkyl, heteroaryl, (heteroaryl)alkyl, halomethyl, CH₂ZR⁶, CH₂OCO(aryl), CH₂OCO(heteroaryl); or CH₂OPO(R⁷)R⁸, where Z is an oxygen or a sulfur atom;
R⁶ is phenyl, substituted phenyl, phenylalkyl, substituted phenylalkyl, heteroaryl, or (heteroaryl)alkyl; and
R⁷ and R⁸ are independently selected from a group consisting of alkyl, cycloalkyl, phenyl, substituted phenyl, phenylalkyl, (substituted phenyl) alkyl, and (cycloalkyl) alkyl; and
X and Y are independently selected from the group consisting of a hydrogen atom, halo, trihalomethyl, amino, protected amino, an amino salt, mono-substituted amino, di-substituted amino, carboxy, protected carboxy, a carboxylate salt, hydroxy, protected hydroxy, a salt of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl;
or a pharmaceutically acceptable salt thereof.

16. The method of claim 12, wherein the protease inhibitor is a compound of formula 3: 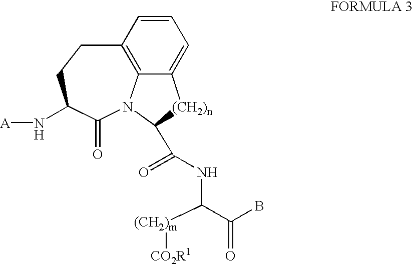
wherein:
n is 1 or 2;
m is 1 or 2;
A is R²CO—, R³—O—CO—, or R⁴SO₂—;
a group of the formula: 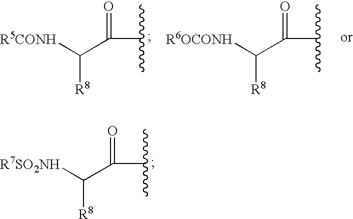
further wherein:
R¹ is a hydrogen atom, alkyl or phenylalkyl;
R² is alkyl, cycloalkyl, (cycloalkyl)alkyl, phenyl, phenylalkyl, substituted phenyl, (substituted phenyl)alkyl, heteroaryl, or (heteroaryl)alkyl;
R³ is alkyl, cycloalkyl, (cycloalkyl)alkyl, phenylalkyl, or (substituted phenyl)alkyl;
R⁴ is alkyl, cycloalkyl, (cycloalkyl)alkyl, phenyl, phenylalkyl, substituted phenyl, (substituted phenyl)alkyl, heteroaryl, or (heteroaryl)alkyl;
R⁵ is alkyl, cycloalkyl, (cycloalkyl)alkyl, phenyl, phenylalkyl, substituted phenyl, (substituted phenyl)alkyl, heteroaryl, or (heteroaryl)alkyl;
R⁶ is alkyl, cycloalkyl, (cycloalkyl)alkyl, phenylalkyl, or (substituted phenyl)alkyl;
R⁷ is alkyl, cycloalkyl, (cycloalkyl)alkyl, phenyl, phenylalkyl, substituted phenyl, (substituted phenyl)alkyl, heteroaryl, or (heteroaryl)alkyl;
R⁸ is an amino acid side chain chosen from the group consisting of natural and unnatural amino acids;
B is a hydrogen atom, a deuterium atom, alkyl, cycloalkyl, (cycloalkyl)alkyl, phenyl, phenylalkyl, substituted phenyl, (substituted phenyl)alkyl, heteroaryl, (heteroaryl)alkyl, or halomethyl;
a group of the formula:
—CH₂XR⁹;
wherein R⁹ is phenyl, substituted phenyl, phenylalkyl, (substituted phenyl)alkyl, heteroaryl, or (heteroaryl)alkyl; and X is an oxygen or a sulfur atom;
a group of the formula:
—CH₂—O—CO-(aryl);
a group of the formula:
—CH₂—O—CO-(heteroaryl);
a group of the formula:
—CH₂—O—PO(R¹⁰)R¹¹
wherein R¹⁰ and R¹¹ are independently selected from a group consisting of alkyl, cycloalkyl, phenyl, substituted phenyl, phenylalkyl and (substituted phenyl) alkyl; and the pharmaceutically-acceptable salts thereof.

17. The method of claim 12, wherein the protease inhibitor is a compound of the formula: 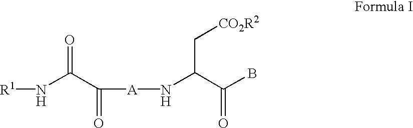
wherein:
A is a natural or unnatural amino acid of Formula IIa-i: 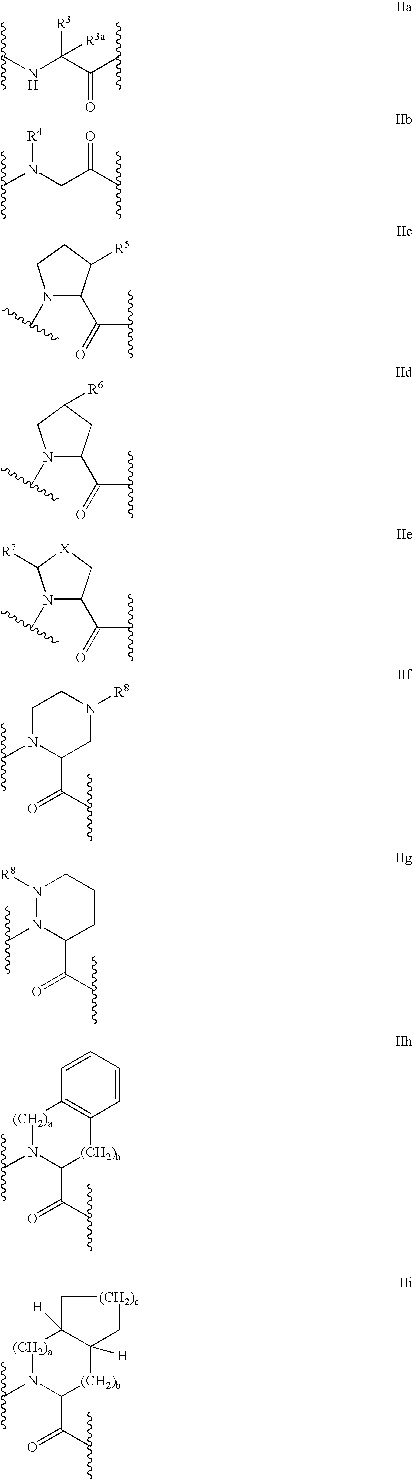
B is a hydrogen atom, a deuterium atom, C₁₋₁₀ straight chain or branched alkyl, cycloalkyl, phenyl, substituted phenyl, naphthyl, substituted naphthyl, 2-benzoxazolyl, substituted 2-oxazolyl, (CH₂)ₙcycloalkyl, (CH₂)ₙphenyl, (CH₂)ₙ(substituted phenyl), (CH₂)ₙ(1 or 2-naphthyl), (CH₂)ₙ(heteroaryl), halomethyl, CO₂R¹², CONR¹³R¹⁴, CH₂ZR¹⁵, CH₂OCO(aryl), CH₂OCO(heteroaryl), or CH₂OPO(R¹⁶)R¹⁷, where Z is an oxygen or a sulfur atom, or B is a group of the Formula IIIa-c: 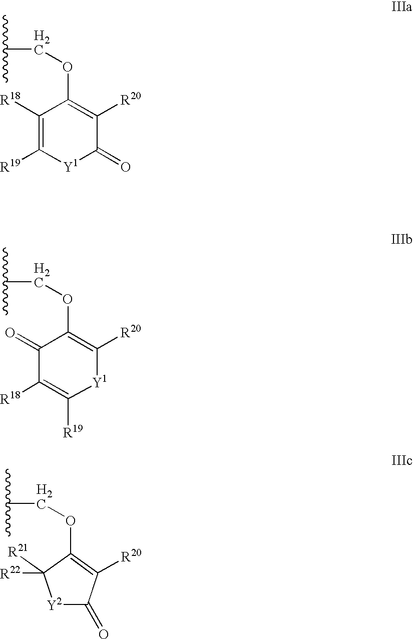
R¹ is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, (heteroaryl)alkyl, R^{1a}(R^{1b})N, [or] R^{1c}O, 2-phenoxyphenyl or 2- or 3-benzylphenyl; and
R² is hydrogen, lower alkyl, cycloalkyl, (cycloalkyl)alkyl, phenylalkyl, or substituted phenylalkyl;
and wherein:
R^{1a} and R^{1b} are independently hydrogen, alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl, with the proviso that R^{1a} and R^{1b} cannot both be hydrogen;
R^{1c} is alkyl, cycloalkyl, (cycloalkyl)alkyl, phenyl, substituted phenyl, phenylalkyl, substituted phenylalkyl, naphthyl, substituted naphthyl, (1 or 2 naphthyl)alkyl, heteroaryl, or (heteroaryl)alkyl;
R³ is C₁₋₆ lower alkyl, cycloalkyl, phenyl, substituted phenyl, (CH₂)ₙNH₂, (CH₂)ₙNHCOR⁹, (CH₂)ₙN(C═NH)NH₂, (CH₂)ₘCO₂R², (CH₂)ₘOR¹⁰, (CH₂)ₘSR¹¹,(CH₂)ₙcycloalkyl,(CH₂)ₙphenyl, (CH₂)ₙ(substituted phenyl), (CH₂)ₙ(1 or 2-naphthyl) or (CH₂)ₙ(heteroaryl), wherein heteroaryl includes pyridyl, thienyl, furyl, thiazolyl, imidazolyl, pyrazolyl, isoxazolyl, pyrazinyl, pyrimidyl, triazinyl, tetrazolyl, and indolyl;
R^{3a} is hydrogen or methyl, or R³ and R^{3a} taken together are —(CH₂)_{d}— where d is an integer from 2 to 6;
R⁴ is phenyl, substituted phenyl, (CH₂)ₘphenyl, (CH₂)ₘ(substituted phenyl), cycloalkyl, or benzofused cycloalkyl;
R⁵ is hydrogen, lower alkyl, cycloalkyl, phenyl, substituted phenyl, (CH₂)ₙcycloalkyl, (CH₂)ₙphenyl, (CH₂)ₙ(substituted phenyl), or (CH₂)ₙ(1 or 2-naphthyl);
R⁶ is hydrogen, fluorine, oxo, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹⁰, SR¹¹ or NHCOR⁹;
R⁷ is hydrogen, oxo, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R⁸ is lower alkyl, cycloalkyl, (CH₂)ₙcycloalkyl, (CH₂)ₙphenyl, (CH₂)ₙ(substituted phenyl), (CH₂)ₙ(1 or 2-naphthyl), or COR⁹;
R⁹ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), (CH₂)ₙ(1 or 2-naphthyl), OR¹², or NR¹³R¹⁴;
R¹⁰ is hydrogen, lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹¹ is lower alkyl, cycloalkyl, phenyl, substituted phenyl, naphthyl, (CH₂)ₙcycloalkyl, (CH₂)ₙphenyl, (CH₂)ₙ(substituted phenyl), or (CH₂)ₙ(1 or 2-naphthyl);
R¹² is lower alkyl, cycloalkyl, (CH₂)ₙcycloalkyl, (CH₂)ₙphenyl, (CH₂)ₙ(substituted phenyl), or (CH₂)ₙ(1 or 2-naphthyl);
R¹³ is hydrogen, lower alkyl, cycloalkyl, phenyl, substituted phenyl, naphthyl, substituted naphthyl, (CH₂)ₙcycloalkyl, (CH₂)ₙphenyl, (CH₂)ₙ(substituted phenyl), or (CH₂)ₙ(1 or 2-naphthyl);
R¹⁴ is hydrogen or lower alkyl;
or R¹³ and R¹⁴ taken together form a five to seven membered carbocyclic or heterocyclic ring, such as morpholine, or N-substituted piperazine;
R¹⁵ is phenyl, substituted phenyl, naphthyl, substituted naphthyl, heteroaryl, (CH₂)ₙphenyl, (CH₂)ₙ(substituted phenyl), (CH₂)ₙ(1 or 2-naphthyl), or (CH₂)ₙ(heteroaryl);
R¹⁶ and R¹⁷ are independently lower alkyl, cycloalkyl, phenyl, substituted phenyl, naphthyl, phenylalkyl, substituted phenylalkyl, or (cycloalkyl)alkyl;
R¹⁸ and R¹⁹ are independently hydrogen, alkyl, phenyl, substituted phenyl, (CH₂)ₙphenyl, (CH₂)ₙ(substituted phenyl), or R¹⁸ and R¹⁹ taken together are —(CH═CH)₂—;
R²⁰ is hydrogen, alkyl, phenyl, substituted phenyl, (CH₂)ₙphenyl, (CH₂)ₙ(substituted phenyl);
R²¹, R²² and R²³ are independently hydrogen, or alkyl;
X is CH₂, (CH₂)₂, (CH₂)₃, or S;
Y¹ is O or NR²³;
Y² is CH₂, O, or NR²³;
a is 0 or 1 and b is 1 or 2, provided that when a is 1 then b is 1;
c is 1 or 2, provided that when c is 1 then a is 0 and b is 1;
m is 1 or 2; and
n is 1, 2, 3 or 4;
or a pharmaceutically acceptable salt thereof.
